(19) United States
(12) Patent Application Publication
(10) Pub. No.: US 2010/0056527 A1
(43) Pub. Date: Mar. 4, 2010

(54) BENZOFURAN COMPOUNDS USEFUL IN THE TREATMENT OF CONDITIONS MEDIATED BY THE ACTION OF PGE2 AT THE EP1 RECEPTOR

(75) Inventors: Andrew John Eatherton, Essex (GB); Gerard Martin Paul Giblin, Essex (GB); Mairi Gibson, Essex (GB); Adrian Hall, Essex (GB); David Nigel Hurst, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited

(21) Appl. No.: 12/527,068

(22) PCT Filed: Feb. 14, 2008
(86) PCT No.: PCT/EP08/51766

§ 371 Date: Aug. 13, 2009

(30) Foreign Application Priority Data
Feb. 16, 2007 (GB) 0703067.9
Sep. 20, 2007 (GB) 0718379.1

(51) Int. Cl. A61K 31/5377; C07D 413/14; C07D 211/32; C07D 231/02; A61K 31/445; A61K 31/415; A61P 25/00

(52) U.S. Cl. 514/236.5; 544/139; 546/199; 548/364.4; 514/320; 514/403

(57) ABSTRACT

A compound of formula (I):

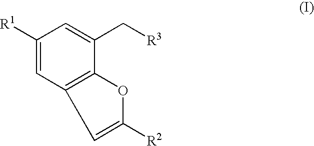

wherein R¹, R², and R³ are as defined in the specification, a process for the preparation of such compounds, pharmaceutical compositions comprising such compounds and the use of such compounds in medicine.

This invention relates to benzofuranyl compounds, to processes for their preparation, to pharmaceutical compositions containing them and to their use in medicine, in particular their use in the treatment of conditions mediated by the action of PGE₂ at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S. Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

The TP (also known as TxA₂) receptor is a prostanoid receptor subtype stimulated by the endogenous mediator thromboxane. Activation of this receptor results in various physiological actions primarily incurred by its platelet aggregatory and smooth muscle constricting effects, thus opposing those of prostacyclin receptor activation.

TP receptors have been identified in human kidneys (G. P. Brown et al, Prostaglandins and other lipid mediators, 1999, 57, 179-188) in the glomerulus and extraglomerular vascular tissue. Activation of TP receptors constricts glomerular capillaries and suppresses glomerular filtration rates (M. D. Breyer et al, Current Opinion in Nephrology and Hypertension, 2000, 9, 23-29), indicating that TP receptor antagonists could be useful for renal dysfunction in glomerulonephritis, diabetes mellitus and sepsis.

Activation of TP receptors induces bronchoconstriction, increase in microvascular permeability, formation of mucosal oedema and mucus secretion, typical characteristic features of bronchial asthma (T. Obata et al, Clinical Review of Allergy, 1994, 12(1), 79-93). TP antagonists have been investigated as potential asthma treatments resulting in, for example, orally active Seratrodast (AA-2414) (S. Terao et al, Yakugaku Zasshi, 1999, 119(5), 377-390). Ramatroban is another TP receptor antagonist currently undergoing phase III clinical trials as an anti-asthmatic compound.

Antagonists at the TP receptor have been shown to have a gastroprotective effect. In rats it has been shown that SQ 33961 and BM 13505 inhibit gastric lesions induced by taurocholate acid, aspirin or indomethacin (E. H. Ogletree et al, Journal of Pharmacology and Experimental Therapeutics, 1992, 263(1), 374-380.

Certain compounds of the present invention also exhibit antagonism at the TP receptor and are therefore indicated to be useful in treating conditions mediated by the action of thromboxane at the TP receptor. Such conditions include those disclosed in WO 2004/039807 (Merck Frosst Canada & Co) which is incorporated herein by reference, and include respiratory diseases e.g. asthma, allergic diseases, male erectile dysfunction, thrombosis, renal disorders and gastric lesions.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006), WO 2006/114313 (2 Nov. 2006), WO 2007/128752 (15 Nov. 2007), WO 2008/006790 (17 Jan. 2008), WO 2008/006793 (17 Jan. 2008), WO 2008/006794 (17 Jan. 2008) and WO 2008/006795 (17 Jan. 2008) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

Naganawa A, Saito T et al: Bioorg Med Chem (2006) 14(16):5562-5577; Naganawa A et al: Bioorg Med Chem (2006) 14(19):6628-6639; Naganawa A et al: Bioorg Med Chem (2006) 14(21):7121-7137; and Naganawa A et al: Bioorg Med Chem (2006) 14(23):7774-7789 disclose various EP₁ antagonists.

A. Hall et al, Bioorg. Med. Chem. Lett., 2007, 17, 4450; S. C. McKeown et al, Bioorg. Med. Chem. Lett., 2007, 17, 1750; A. Hall et al, Bioorg. Med. Chem. Lett., 2007, 17, 1200; A. Hall et al, Bioorg. Med. Chem. Lett., 2007, 17, 916; A. Hall et al, Bioorg. Med. Chem. Lett., 2007, 17, 732; G. M. P. Giblin et al, Bioorg. Med. Chem. Lett., 2007, 17, 385-389; S. C. McKeown et al, Bioorg. Med. Chem. Lett., 2006, 16 (18), 4767-4771; A. Hall et al, Bioorg. Med. Chem. Lett., 2006, 16 (14), 3657-3662; and A. Hall et al, Bioorg. Med. Chem. Lett., 2006, 16 (10), 2666-2671 relate to EP₁ receptor antagonist compounds.

Accordingly the present invention provides one or more chemical entities selected from compounds of formula (I):

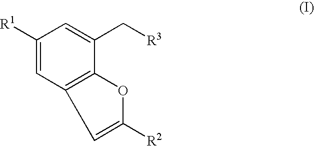

wherein:
R¹ is hydrogen, halogen, CN, CF₃ or SO₂CH₃;
R² is thienyl, thiazolyl, 1-methylimidazolyl, CH₂phenyl, phenyl optionally substituted by Cl, F or CN, or pyridyl optionally substituted by halogen;

### R³ is

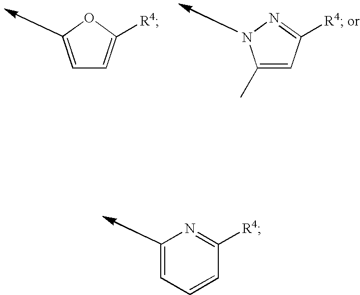

R⁴ is CO₂H, NHCO₂R⁵, CONR^{6a}R^{6b}, NHCOR⁷, NHCONR⁸R⁹, CONHSO₂R¹⁰, imidazole or tetrazole;
or R⁴ is an imidazole ring fused to give an optionally substituted bicyclic or tricyclic ring system;
R⁵ represents C₂₋₆ alkyl, or CH₂-heterocyclyl;
R^{6a} represents hydrogen; and
R^{6b} represents hydrogen; indane; NR¹¹R¹²; C₁₋₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle or CONH-aliphatic heterocycle;
or R^{6a} and R^{6b} together with the nitrogen atom to which they are attached is an optionally substituted aliphatic heterocycle;
R⁷ is C₁₋₆alkyl; CH₂N(CH₃)₂; or optionally substituted (CH₂)ₙaliphatic heterocycle wherein n is 0, or 1;
R⁸ is hydrogen or C₁₋₄alkyl;
R⁹ is C₁₋₄alkyl;
R¹⁰ is C₁₋₄alkyl, aryl or heteroaryl;
R¹¹ is hydrogen or C₁₋₄alkyl; and
R¹² is hydrogen or C₁₋₄alkyl;
or derivatives thereof.

In one aspect

R¹ is hydrogen, halogen, CN, or SO₂CH₃;
R² is thienyl, thiazolyl, 1-methylimidazolyl, CH₂phenyl, phenyl optionally substituted by Cl, F or CN, or pyridyl optionally substituted by halogen;

### R³ is

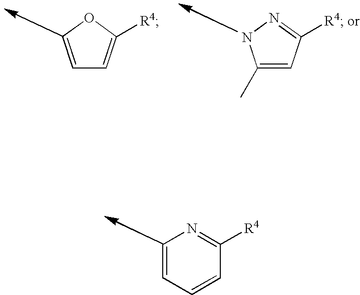

R⁴ is CO₂H, NHCO₂R⁵, CONR^{6a}R^{6b}, NHCOR⁷, NHCONR⁸R⁹, imidazole or tetrazole;
or R⁴ is an imidazole ring fused to give an optionally substituted bicyclic or tricyclic ring system;
R⁵ represents C₂₋₆ alkyl, or CH₂-heterocyclyl;
R^{6a} represents hydrogen; and
R^{6b} represents hydrogen; indane; NR¹¹R¹²; C₁₋₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle;
or R^{6a} and R^{6b} together with the nitrogen atom to which they are attached is an optionally substituted aliphatic heterocycle;
R⁷ is C₁₋₆alkyl; CH₂N(CH₃)₂; or optionally substituted (CH₂)ₙaliphatic heterocycle wherein n is 0, or 1;
R⁸ is hydrogen or C₁₋₄alkyl;
R⁹ is C₁₋₄alkyl;
R¹¹ is hydrogen or C₁₋₄alkyl; and
R¹² is hydrogen or C₁₋₄alkyl.

Suitably R¹ is Cl, CN, or SO₂CH₃. In one aspect R¹ is Cl.

In one embodiment, R² is phenyl.

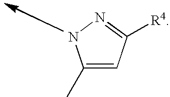

Suitably R³ is

Suitably R⁴ is CO₂H, NHCO₂R⁵, CONR^{6a}R^{6b}, NHCOR⁷ or NHCONR⁸R⁹. In one aspect R⁴ is CO₂H, CONR^{6a}R^{6b}, or NHCOR⁷. In another aspect R⁴ is CONR^{6a}R^{6b}, or NHCOR⁷. In yet another aspect R⁴ is CONHR^{6b}.

In one embodiment R⁵ is C₂₋₆ alkyl, e.g. tert-butyl and iso-butyl. In one aspect R⁵ is tert-butyl.

In one aspect R^{6b} represents hydrogen; indane; NR¹¹R¹²; C₁₋₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle.

In another aspect R^{6b} is hydrogen; indane; N(CH₃)₂, C₁₋₆alkyl optionally substituted by F, CF₃, OH, OC₁₋₄alkyl or NR⁸R⁹; phenyl optionally substituted by CH₂NR⁸R⁹, or optionally substituted CH₂aliphatic heterocycle; optionally substituted (CH₂)ₙaliphatic heterocycle wherein n is 0, 1 or 2; or pyridine optionally substituted by CH₂aliphatic heterocycle; wherein R⁸ is hydrogen or C₁₋₄alkyl and R⁹ is C₁₋₄alkyl.

When R^{6b} is optionally substituted aliphatic heterocycle, preferably it is linked via a ring nitrogen atom.

When R^{6b} is pyridine optionally substituted by CH₂aliphatic heterocycle, preferably the aliphatic heterocycle is linked via a ring nitrogen atom.

Suitably R^{6b} is hydrogen; indane; N(CH₃)₂; CH₂CF₃; CH₂CH₂OH; CH₂CH₂F; CH₂CH₂OCH₃; CH₂CH₂N(CH₃)₂; CH₂CH₂CH₃; CH₂CH(OH)CF₃; CH(CH₃)₂; cyclopropyl; CH₂cyclopropyl; C(CH₃)₃; CH(CH₃)₂CH₂OH; cyclobutyl; CH₂C(CH₃)₃; CH₂cyclobutyl; phenyl optionally substituted by CH₂NHCH₂CH₃, CH₂NHCH(CH₃)₂, CH₂pyrrolidine, CH₂piperidine, or CH₂morpholine; morpholine; piperidine optionally substituted by OH; tetrahydropyran; pyrrolidine optionally substituted by COCH₃; CH₂tetrahydrofuran; CH₂piperidine optionally substituted by C₁₋₂alkyl; CH₂tetrahydrofuran; CH₂CH₂morpholine; or pyridine optionally substituted with CH₂piperazine or CONHCH₂piperidine.

In one aspect R^{6b} is tert-butyl or CH₂piperidine optionally substituted by CH₂CH₃. In another aspect R^{6b} is tert-butyl.

When R^{6a} and R^{6b} together with the nitrogen atom to which they are attached form an optionally substituted aliphatic heterocycle, suitable aliphatic heterocycles include piperidine, morpholine and piperazine all of which may be substituted with, e.g. C₁₋₄alkyl. Particular aliphatic heterocycles include morpholine, piperidine and 4-methyl piperazine.

When the group R⁷ contains an optionally substituted aliphatic heterocycle, suitably the heterocycle is tetrahydropyran, piperidine, tetrahydrofuran, pyrrolidine or azetidine. Suitable optional substituents include one or two substituents selected from C₁₋₄alkyl, ═O, F, CH₂CF₃ or COCH₃. In one aspect R⁷ is C₁₋₆alkyl; CH₂N(CH₃)₂; optionally substituted (CH₂)ₙaliphatic heterocycle wherein n is 0 or 1 or 2 and the aliphatic heterocycle is selected from tetrahydropyran, piperidine, tetrahydrofuran, pyrrolidine and azetidine and is optionally substituted with one or more substituents selected from C₁₋₄alkyl, ═O, F, CH₂CF₃ or COCH₃.

Suitably R⁷ is isopropyl; CH₂N(CH₃)₂; azetidine optionally substituted by COCH₃; pyrrolidine optionally substituted by one or more substituents selected from C₁₋₃alkyl, ═O, and COCH₃; tetrahydrofuran; tetrahydropyran; piperidine optionally substituted by F or CH₂CF₃; CH₂pyrrolidine wherein the pyrrolidine ring is optionally substituted by ═O; or CH₂piperidine. In one aspect R⁷ is 1-methylpyrrolidin-2-on-4-yl.

In one aspect R⁸ is hydrogen.

In one aspect R⁹ is C₁₋₄alkyl, e.g. tert-butyl.

Compounds of formula (I) include the compounds of examples 1 to 156 and derivatives thereof.

An example of a compound of formula (I) is 1-[(5-chloro-2-phenyl-1-benzofuran-7-yl)methyl]-5-methyl-N-4-morpholinyl-1H-pyrazole-3-carboxamide or a derivative thereof, particularly a pharmaceutically acceptable derivative thereof.

A further example of a compound of formula (I) is 1-[(5-chloro-2-phenyl-1-benzofuran-7-yl)methyl]-N-(1,1-dimethylethyl)-5-methyl-1H-pyrazole-3-carboxamide or a derivative thereof, particularly a pharmaceutically acceptable derivative thereof.

A yet further example of a compound of formula (I) is N-{1-[(5-Chloro-2-phenyl-1-benzofuran-7-yl)methyl]-5-methyl-1H-pyrazol-3-yl}-2-(4-piperidinyl)acetamide or a derivative thereof, particularly a pharmaceutically acceptable derivative thereof.

Another example of a compound of formula (I) is N-{1-[(5-chloro-2-phenyl-1-benzofuran-7-yl)methyl]-5-methyl-1H-pyrazol-3-yl}-1-methyl-5-oxo-3-pyrrolidinecarboxamide or a derivative thereof, particularly a pharmaceutically acceptable derivative thereof.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine. The term “aliphatic heterocyclyl” as a group or as part of a group means an aliphatic five or six membered ring which contains 1 or 2 heteroatoms selected from nitrogen, oxygen or sulfur and is unsubstituted or substituted by, for example, up to three substituents, preferably one or two substituents.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

Compounds of formula (I) can be prepared as set forth in the following schemes and in the Examples. The following processes form another aspect of the present invention.

For example, compounds of formula (I) wherein R² is thienyl, thiazolyl, 1-methylimidazolyl, phenyl optionally substituted by Cl, F or CN, or pyridyl optionally substituted by halogen (hereinafter R^{2a}), R³ is

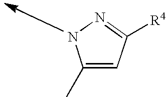

and R⁴ is CO₂H may be prepared by the general route shown in Scheme I below:

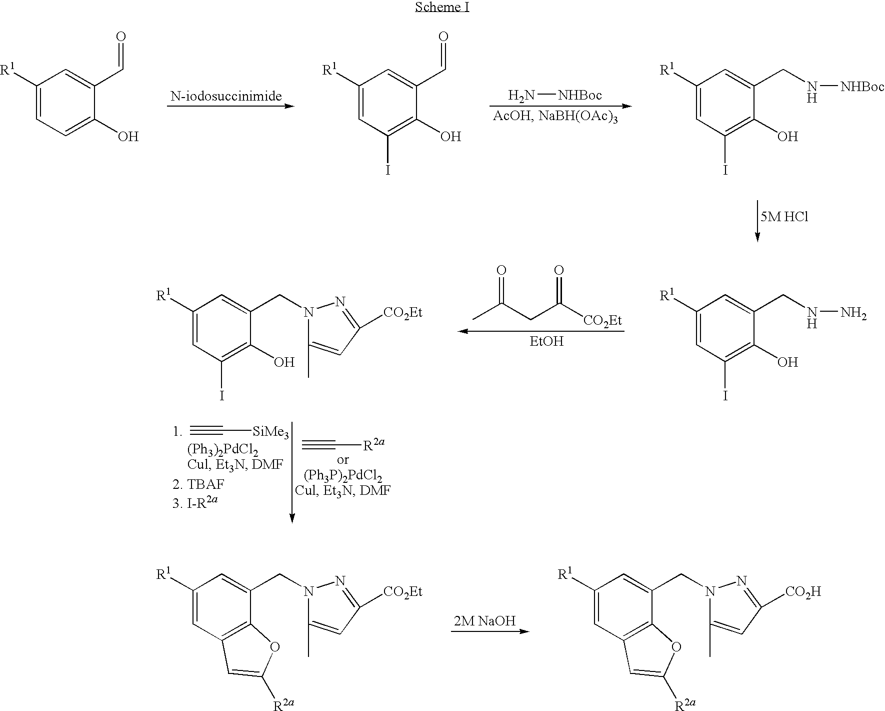

wherein R¹ is as defined hereinabove for compounds of formula (I), and R^{2a} is thienyl, thiazolyl, 1-methylimidazolyl, phenyl optionally substituted by Cl, F or CN, or pyridyl optionally substituted by halogen, DMF is N,N-dimethylformamide and TBAF is tetrabutylammonium fluoride.

It will be appreciated that the synthesis of the pyrazole ring may result in the formation of regioisomers. Separation of such regioisomers may be carried out using known techniques such as chromatography or recrystallisation.

Alternatively, compounds of formula (I) wherein R³ is the group

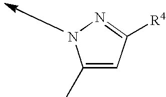

and R⁴ is CO₂H may be prepared using the general route shown in Scheme II below.

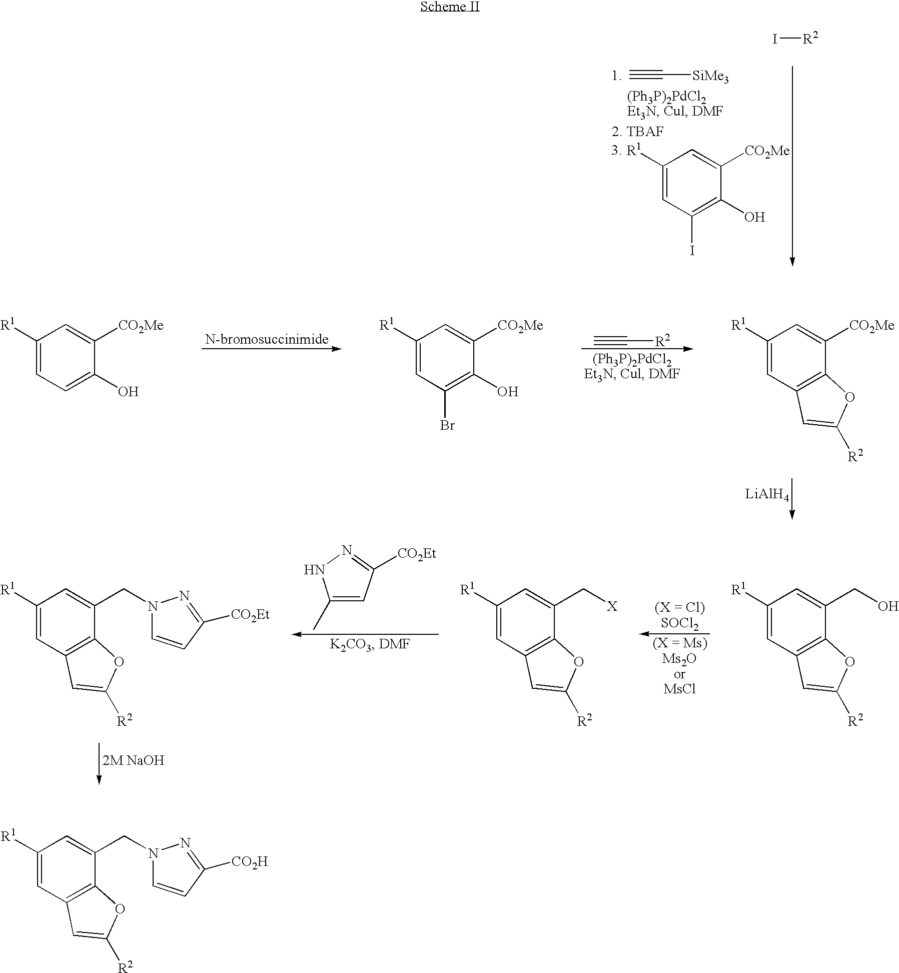

Wherein R¹ and R² are as defined for compounds of formula (I), X is Cl or mesylate, TBAF is tetrabutylammonium fluoride and DMF is N,N-dimethylformamide.

Compounds of formula (I) wherein R³ is

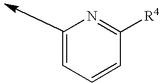

dud R⁴ is CO₂H may be prepared using the general route described in Scheme III.

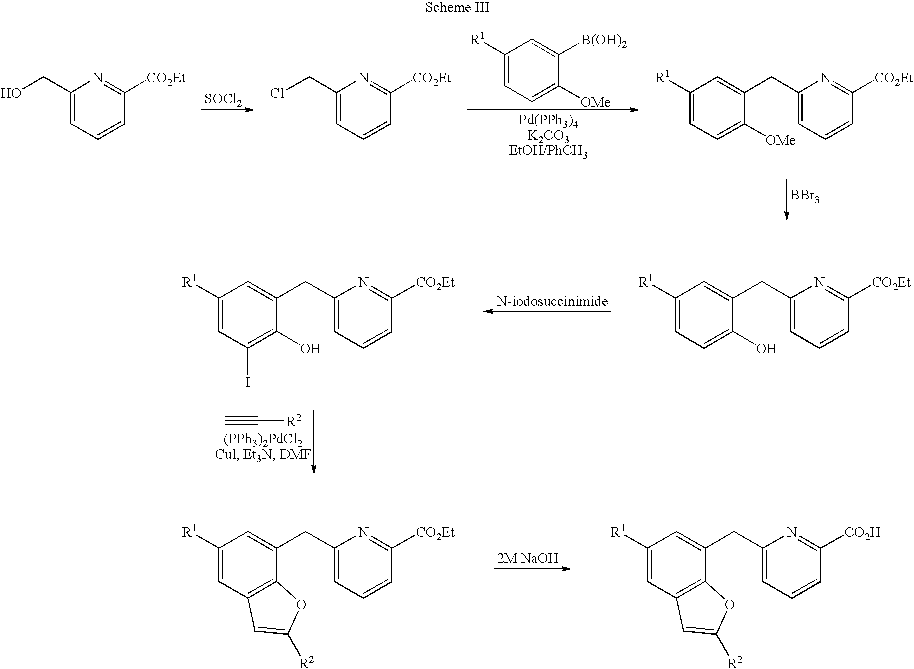

wherein R¹ and R² are as defined for compounds of formula (I), DMF is N,N-dimethylformamide and TBAF is tetrabutylammonium fluoride.

Compounds of formula (I) wherein R³ is

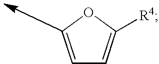

and R⁴ is CO₂H may be prepared using analogous routes to those described above in Scheme III.

Compounds of formula (I) wherein R³ is

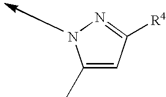

and R⁴ is CONHR wherein R is a group R^{6b} or a group which may be converted to a group R^{6b} (wherein R^{6b} is as defined for compounds of formula (I)) may be prepared in accordance with Scheme IV.

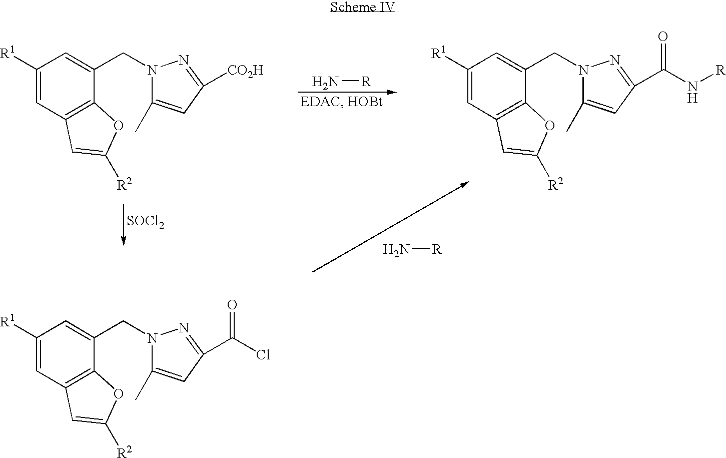

wherein R¹ and R² are as defined for compounds of formula (I), R is a group R^{6b} as defined for compounds of formula (I) or a group that can be converted to a group R^{6b}, EDAC is 1-(3-dimethylaminopropyl)-3-ethylcarbodiimide hydrochloride and HOBt is 1-hydroxybenzotriazole.

Compounds of formula (I) wherein R³ is

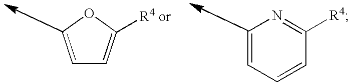

and R⁴ is CONHR wherein R is a group R^{6b} as defined for compounds of formula (I) or a group that may be converted to a group R^{6b} may also be prepared in accordance with the procedure of Scheme IV.

Compounds of formula (I) wherein R³ is

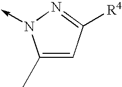

and R⁴ is —CONHPhCH₂NR¹¹R¹² wherein R¹¹ and R¹² are as defined for compounds of formula (I) may be prepared in accordance with Scheme V.

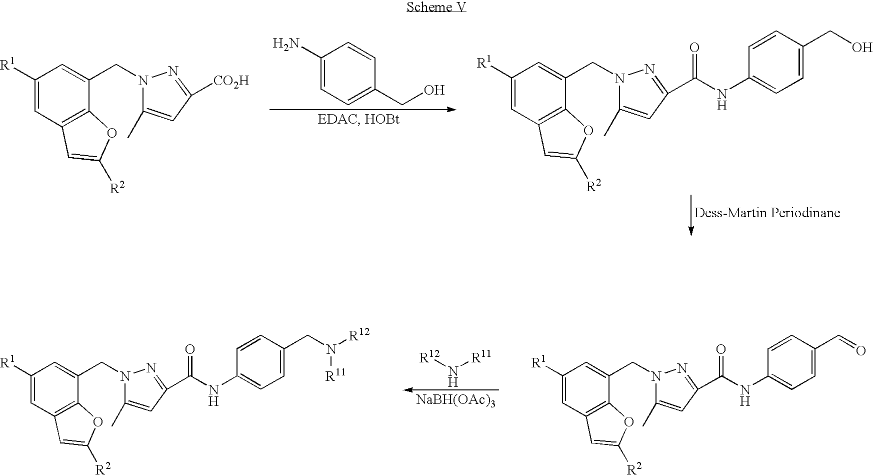

wherein R¹, R², R¹¹ and R¹² are as defined for compounds of formula (I), EDAC is 1-(3-dimethylaminopropyl)-3-ethylcarbodiimide hydrochloride and HOBt is 1-hydroxybenzotriazole. Dess-Martin periodinane is 1,1,1-triacetoxy-1,1-dihydro-1,2-benziodoxol-3(1H)-one.

Compounds of formula (I) wherein R³ is

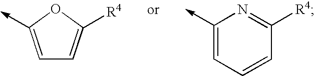

and R⁴ is —CONHPhCH₂NR¹¹R¹², wherein R¹¹ and R¹² are as defined for compounds of formula (I), may also be prepared using routes analogous to those described in Scheme V.

Compounds of formula (I) wherein R⁴ is pyridine optionally substituted by CH₂-aliphatic heterocycle may be prepared from a formyl piperidinyl intermediate of the formula:

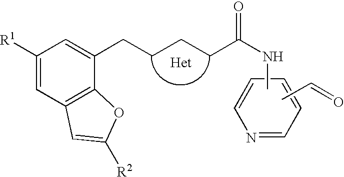

wherein R¹ and R² are as defined for compounds of formula (I) and “Het” represents the ring systems as defined for R³;
by reacting with an appropriate amine in the presence of NaBH(OAc)₃ in an analogous manner to that described above in Scheme V.

The formyl piperidinyl intermediate may be prepared from a compound of formula:

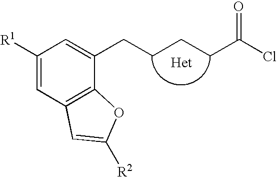

wherein R¹ and R² are as defined for compounds of formula (I) and “Het” represents the ring systems as defined for R³;
by reaction with the appropriate ethenylpyridinamine under suitable conditions, e.g. by use of triethylamine in dry dichloromethane, to give a compound of formula:

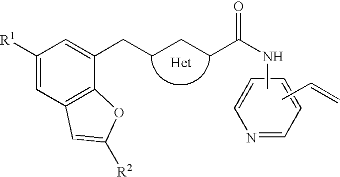

the ethenyl group may then be converted to a formyl group by conventional methods, e.g. osmium tetroxide and sodium periodate, to give the required formyl piperidinyl intermediate.

Compounds of formula (I) wherein R³ is

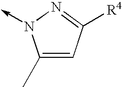

and R⁴ is CONH-pyridyl-CONHR wherein R is aliphatic heterocycle or a group that can be converted to aliphatic heterocycle may be prepared using the route described in Scheme VI below:

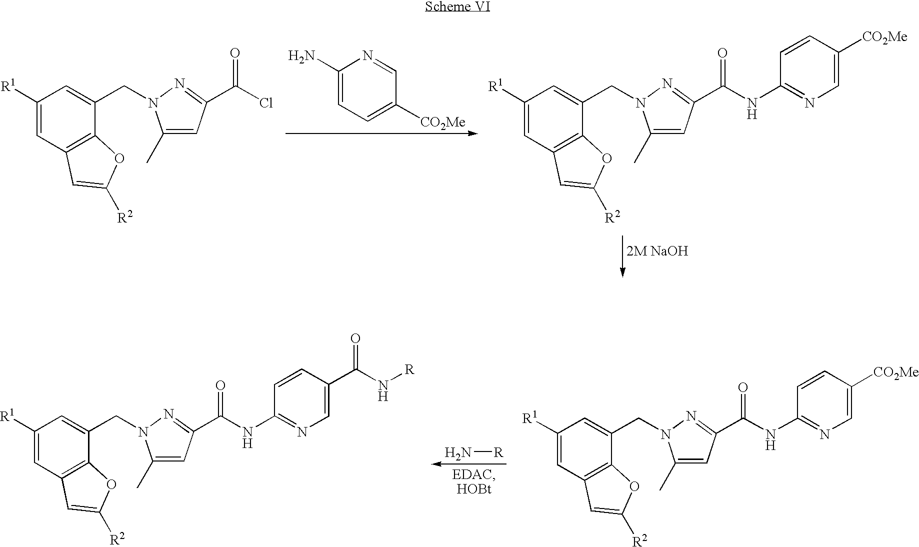

wherein R¹ and R² are as defined for compounds of formula (I), R is an aliphatic heterocyclic group or a group that may be converted to an aliphatic heterocyclic group, EDAC is 1-(3-dimethylaminopropyl)-3-ethylcarbodiimide hydrochloride and HOBt is 1-hydroxybenzotriazole.

Compounds of formula (I) wherein R³ is

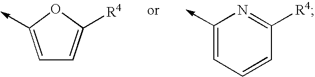

and R⁴ is CONH-pyridyl-CONHR wherein R is an aliphatic heterocyclic group or a group that may be converted to an aliphatic heterocyclic group may be prepared from appropriate starting materials using routes analogous to those described in Scheme VI.

Compounds of formula (I) wherein R³ is

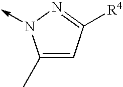

and R⁴ is CONHR^{6b} wherein R^{6b} is optionally substituted (CH₂)ₙaliphatic heterocycle wherein n is 0, 1, or 2 (such as optionally substituted (CH₂)ₙpiperidine) may be prepared using the route described in Scheme VII below:

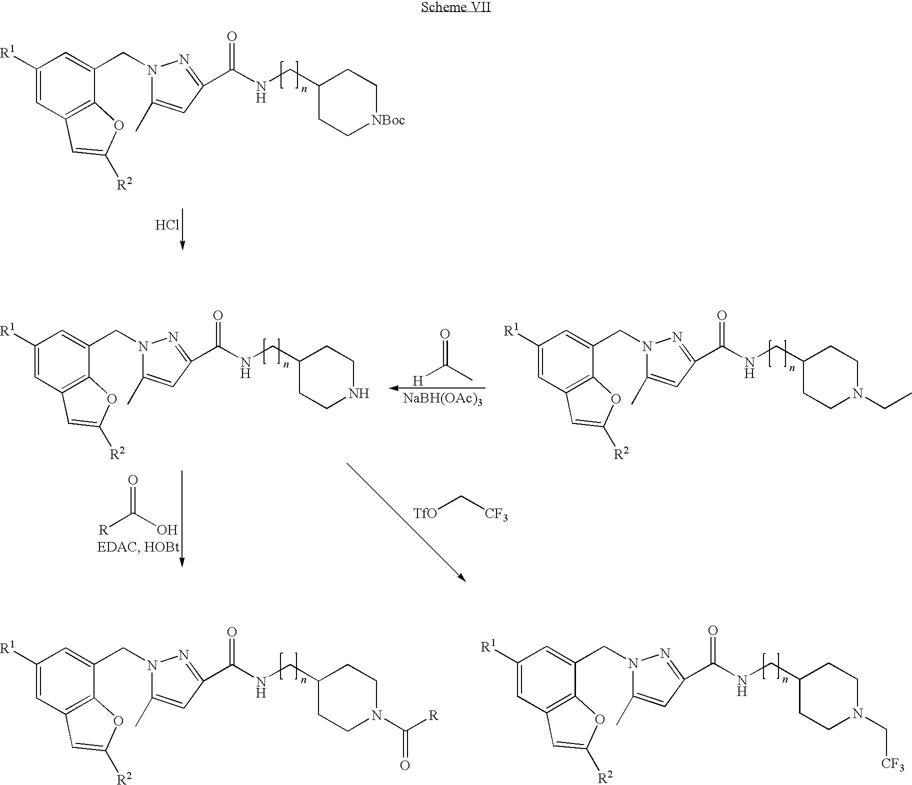

wherein R¹ and R² are as defined for compounds of formula (I), n is 0, 1, or 2 and R is C₁₋₂alkyl, EDAC is 1-(3-dimethylaminopropyl)-3-ethylcarbodiimide hydrochloride and HOBt is 1-hydroxybenzotriazole.

Compounds of formula (I) wherein R³ is

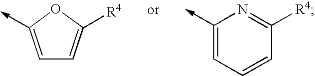

and R⁴ is CONHR^{6b} wherein R^{6b} is optionally substituted (CH₂)ₙaliphatic heterocycle wherein n is 0, 1, or 2 may be prepared from appropriate starting materials using analogous routes to those described in Scheme VII.

Compounds of formula (I) wherein R³ is

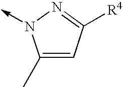

and R is NHCO₂R⁵, NHCOR⁷, or NHCONR⁸R⁹ and R⁵, R⁷, R⁸, and R⁹ are as defined for compounds of formula (I) may be prepared using the routes described in Scheme VIII below:

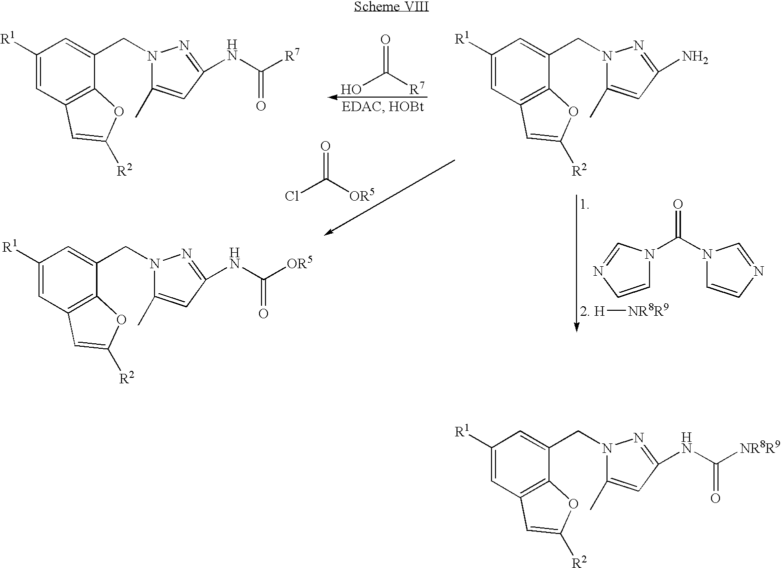

wherein R¹, R², R⁵, R⁷, R⁸, and R⁹ are as defined for compounds of formula (I), EDAC is 1-(3-dimethylaminopropyl)-3-ethylcarbodiimide hydrochloride and HOBt is 1-hydroxybenzotriazole.

Intermediates of the formula

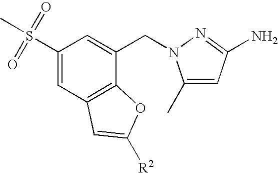

wherein R² is as defined for compounds of formula (I) may be prepared in accordance with the process of Scheme IX:

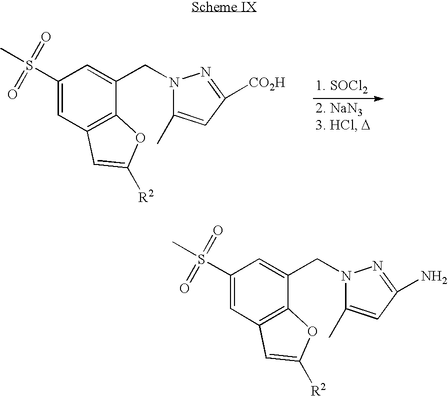

Intermediates of the formula

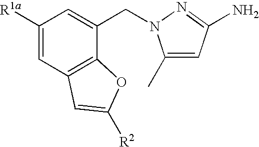

wherein R^{1a} is hydrogen, halogen, CN, or CF₃ and R² is as defined for compounds of formula (I) may be prepared in accordance with the procedure of Scheme X.

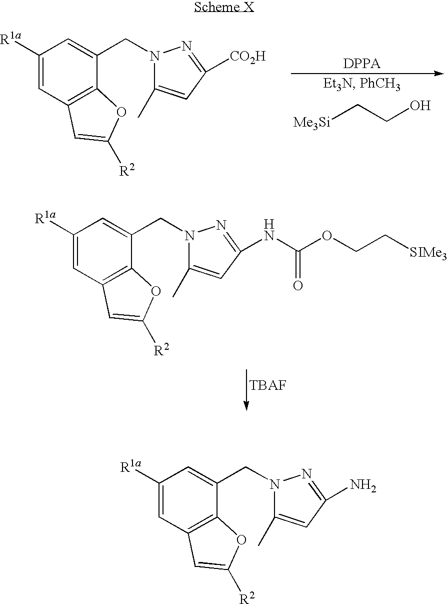

Wherein R² is as defined for compounds of formula (I), R^{1a} is hydrogen, halogen, CN, or CF₃, DPPA diphenylphosphoryl azide and TBAF is tetrabutylammonium fluoride.

Compounds of formula I wherein R³ is

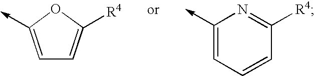

R⁴ is NHCO₂R⁵, NHCOR⁷, or NHCONR⁸R⁹ and R⁵, R⁷, R⁸, and R⁹ are as defined for compounds of formula (I) may be prepared using routes analogous to those described in Schemes VIII, IX and X.

It will be recognised to those skilled in the art that the compounds of formula (I) can be derived from compounds of formula (I) wherein R⁴ is CO₂H. Compounds of formula (I) wherein R⁴ is an amide group (e.g. —CONR^{6a}R^{6b}, and —CONHSO₂R¹⁰) may be prepared by activation of the carboxylic acid of a compound of formula (I) wherein R⁴ is CO₂H, for example by forming the acid chloride (for example by reaction of the carboxylic acid with thionyl chloride) followed by reaction with an amine or a sulfonamide respectively. Compounds of formula (I) wherein R⁴ is —NHCO₂R⁵ may be accessed by using the Curtius reaction (P. A. S. Smith, Org. React. 3, 337-449 (1946) and J. H. Saunders, R. J. Slocombe, Chem. Rev. 43, 205 (1948)).

A carboxylic acid group may be converted to an imidazole group by a sequence of well known functional group transformations such as those described in A. R. Katritzky, C. W. Rees ‘Comprehensive Heterocyclic Chemistry’, Pergamon (1984). Tetrazoles may be formed from carboxylic acids by converting the carboxylic acid to the primary amides, for example by reaction with oxalyl chloride followed by ammonia, followed by dehydration of the amide to the nitrile, for example by heating in phosphorous oxychloride, followed by reaction with azide.

Compounds of formula (I) wherein R⁴ is an imidazole moiety fused to give an optionally substituted bicyclic or tricyclic ring system may be prepared from intermediates of the formula:

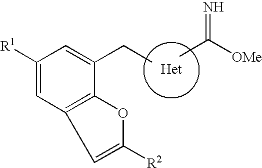

wherein R¹ and R² are as defined for compounds of formula (I) and “het” represents the furan, pyrazole or pyridyl ring systems as defined for R³.

These compounds may be prepared from the corresponding compound of formula (I) wherein R⁴ is CO₂H by known methods. Suitable methods include the reaction of the carboxylic acid with thionyl chloride then ammonia, then phosphorus oxychloride, then sodium methoxide in methanol. These intermediates may then be converted to compounds of formula (I) wherein R⁴ is an imidazole moiety fused to give an optionally substituted bicyclic or tricyclic ring system following the methods described in, for example, A. Czarny et al, J. Het. Chem., 1996, 33(4), 1393-1398.

Alternatively, compounds wherein R⁴ is -benzimidazolyl may typically be prepared by reacting a compound of formula (I) wherein R⁴ is CO₂H with 1,2-phenylenediamine or a suitably substituted analogue, N-(3-dimethylaminopropyl)-N′-ethylcarbodiimide hydrochloride and HOBT in the presence of a suitable solvent, such as dichloromethane followed by dehydration according to standard conditions known to the skilled person. Suitable diamines are commercially available, or may be prepared by known methods.

Compounds of formula (I) wherein R⁴ is -benzimidazolyl may also be prepared from the reaction of a suitable diamine with an intermediate of the formula:

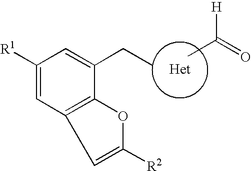

wherein R¹ and R² are as defined for compounds of formula (I) and “Het” represents the ring systems as defined for R³. These intermediates may be prepared from compounds of formula (I) wherein R⁴ is CO₂H by known methods, for example by reaction with lithium aluminum hydride in a suitable solvent, e.g. THF to give the corresponding methanol, followed by conversion to the corresponding carbaldehyde using Dess-Martin periodinane.

Compounds of formula (I) wherein R⁴ is benzimidazole may be functionalised on the benzimidazole ring using methods known in the art.

Accordingly the present invention also provides a process for the preparation of a compound of formula (I) or a derivative thereof:

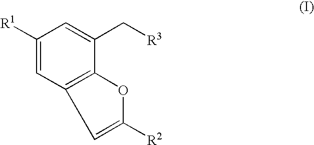

wherein:
R¹ is hydrogen, halogen, CN, CF₃ or SO₂CH₃;
R² is thienyl, thiazolyl, 1-methylimidazolyl, CH₂phenyl, phenyl optionally substituted by Cl, F or CN, or pyridyl optionally substituted by halogen;

### R³ is

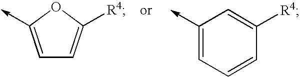

R⁴ is CO₂H, NHCO₂R⁵, CONR^{6a}R^{6b}, NHCOR⁷, NHCONR⁸R⁹, CONHSO₂R¹⁰, imidazole or tetrazole;
or R⁴ is an imidazole ring fused to give an optionally substituted bicyclic or tricyclic ring system;
R⁵ represents C₂₋₆ alkyl, or CH₂-heterocyclyl;
R^{6a} represents hydrogen; and
R^{6b} represents hydrogen; indane; NR¹¹R¹²; C₁₋₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle or CONH-aliphatic heterocycle;
or R^{6a} and R^{6b} together with the nitrogen atom to which they are attached is an optionally substituted aliphatic heterocycle;
R⁷ is C₁₋₆alkyl; CH₂N(CH₃)₂; or optionally substituted (CH₂)ₙaliphatic heterocycle wherein n is 0, or 1;
R⁸ is hydrogen or C₁₋₄alkyl;
R⁹ is C₁₋₄alkyl;
R¹⁰ is C₁₋₄alkyl, aryl or heteroaryl;
R¹¹ is hydrogen or C₁₋₄alkyl; and
R¹² is hydrogen or C₁₋₄alkyl;
comprising:
reacting a compound of formula:

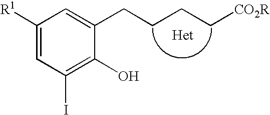

wherein R is methyl or ethyl; R¹ is as defined for compounds of formula (I); and Het is:

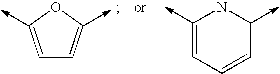

with a compound of the formula:

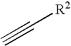

wherein R² is as defined for compounds of formula (I);
and if required, and in any order;
converting one group R⁴ to another group R⁴; and/or
effecting deprotection; and/or
forming a derivative thereof.

The present invention also provides a process for the preparation of a compound of formula (I) or a derivative thereof:

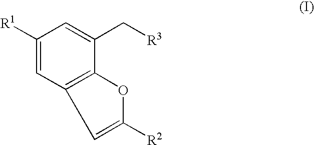

wherein:
R¹ is hydrogen, halogen, CN, CF₃ or SO₂CH₃;
R² is thienyl, thiazolyl, 1-methylimidazolyl, CH₂phenyl, phenyl optionally substituted by Cl, F or CN, or pyridyl optionally substituted by halogen;

### R³ is

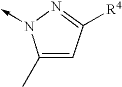

R⁴ is CO₂H, NHCO₂R⁵, CONR^{6a}R^{6b}, NHCOR⁷, NHCONR⁸R⁹, CONHSO₂R¹⁰, imidazole or tetrazole;
or R⁴ is an imidazole ring fused to give an optionally substituted bicyclic or tricyclic ring system;
R⁵ represents C₂₋₆ alkyl, or CH₂-heterocyclyl;
R^{6a} represents hydrogen; and
R^{6b} represents hydrogen; indane; NR¹¹R¹²; C₁₋₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle or CONH-aliphatic heterocycle;
or R^{6a} and R^{6b} together with the nitrogen atom to which they are attached is an optionally substituted aliphatic heterocycle;
R⁷ is C₁₋₆alkyl; CH₂N(CH₃)₂; or optionally substituted (CH₂)ₙaliphatic heterocycle wherein n is 0, or 1;
R⁸ is hydrogen or C₁₋₄alkyl;
R⁹ is C₁₋₄alkyl;
R¹⁰ is C₁₋₄alkyl, aryl or heteroaryl;
R¹¹ is hydrogen or C₁₋₄alkyl; and
R¹² is hydrogen or C₁₋₄alkyl;
comprising:
reacting a compound of formula:

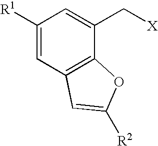

wherein R¹ and R² are as defined for compounds of formula (I); and X is a leaving group such as chloro or mesylate;
with a compound of the formula:

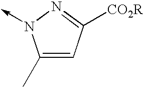

wherein R is methyl or ethyl;
and if required, and in any order;
converting one group R⁴ to another group R⁴; and/or
effecting deprotection; and/or
forming a derivative thereof.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anemia, Hodgkin's disease, scleredoma, myasthenia gravis, multiple sclerosis, sarcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cachexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; hemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration.

The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; brakykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

In addition to activity at the EP₁ receptor, certain compounds of the present invention and pharmaceutically acceptable derivatives thereof exhibit antagonism of the TP receptor and are therefore indicated to be useful in treating conditions mediated by the action of thromboxane at the TP receptor. Conditions mediated by the action of thromboxane at the TP receptor include renal disorders, asthma, or gastric lesions.

In certain situations it is envisaged that the administration of a compound exhibiting antagonism of TP receptors in combination with a compound exhibiting antagonism of EP₁ receptors may be advantageous.

Certain compounds of the invention are selective for EP₁ over EP₃.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

It will be appreciated to those skilled in the art that where compounds are named as hydrochloride salts the stoichiometry of the isolated reaction products is undetermined due to the nature of their preparation. Compounds have therefore been named as hydrochlorides and denoted as xHCl, where x is 0-3 and represents the stoichiometry of said salt. Similar considerations apply to compounds herein named as mesylate or trifluoroacetate salts.

### Abbreviations

AcOH, acetic acid, Bn (benzyl), Bu, Pr, Me, Et (butyl, propyl, methyl, ethyl), DBU (1,8-diazabicyclo[5.4.0]undec-7-ene), DMSO (dimethyl sulfoxide), DCM/MDC (dichloromethane), DME (ethylene glycol dimethyl ether), DMF (N,N-dimethylformamide), EDTA (ethylenediaminetetraacetic acid), EtOAc (ethyl acetate), EtOH (ethanol), EDAC (1-(3-dimethylaminopropyl)-3-ethylcarbodiimide hydrochloride), HOBT/HOBt (1-hydroxybenzotriazole), HPLC (High pressure liquid chromatography), IPA (isopropanol), LCMS (Liquid chromatography/Mass spectroscopy), MDAP (Mass Directed Auto Preparation), MeOH (methanol), ML (mother liquor), NBS (N-bromosuccinimide), NMR (Nuclear Magnetic Resonance (spectrum)), NMP (n-methylpyrrolidone), Ph (phenyl), pTSA (para-toluene sulfonic acid), RT/Rt (retention time), SM (starting material), SPE (Solid Phase Extraction—silica cartridge chromatography), TBAF (tetrabutylammonium fluoride), TBME (tertiary butyl methyl ether), TEA (triethylamine), THF (tetrahydrofuran), s, d, dd, t, q, m, br (singlet, doublet, double doublet, triplet, quartet, multiplet, broad.)

### Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation).

The term “Biotage” when used herein refers to commercially available pre-packed silica gel cartridges.

### LCMS

The following LCMS conditions were used during the preparation of the examples.

### Software

Waters MassLynx version 4.0 SP2

### Column

The column used is a Waters Atlantis, the dimensions of which are 4.6 mm×50 mm. The stationary phase particle size is 3 m.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Description 1 (D1)

### 1-{[(1,1-Dimethylethyl)oxy]carbonyl}-4-fluoro-4-piperidinecarboxylic acid

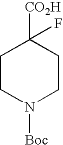

Solution of 4-fluoro-1-(1,1-dimethylethyl)-1,4-piperidine dicarboxylic acid-4-methyl ester (1.00 g, 4.02 mmol) in EtOH (16 ml) was stirred at room temperature. 2M NaOH (5.0 ml, 10.05 mmol) was added and the solution heated to 60° C. for 5¼ hours. After this time, the solution was left to cool to room temperature overnight. The solution was then acidified using 2M HCl and the organics were extracted into EtOAc (×2). The combined organics were dried over MgSO₄, filtered and concentrated under reduced pressure to give a white coloured solid (0.767 g)

### Description 2 (D2)

### Ethyl 1-(2,2,2-trifluoroethyl)-4-piperidinecarboxylate

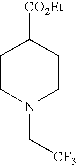

Solution of piperidine-4-carboxylic acid ethyl ester (1.00 g, 6.36 mmol) in EtOH (12 ml) was stirred at room temperature. 2,2,2-trifluoroethyl trifluoromethane sulfonate (0.737 g, 3.18 mmol) was added. NaHCO₃ (0.534 g, 6.36 mmol) was then added. Solution was stirred for 18 hours (overnight) at reflux. After this time, solution was allowed to cool to room temperature and then the solvent was removed under reduced pressure. Residue was partitioned between DCM and water. Organics were washed with water, then dried over MgSO₄, filtered and concentrated under reduced pressure to give an oily solid. The residue was chromatographed [SiO₂, Hexane/EtOAc, 25-50%] to give the title compound (0.632 g).

### Description 3 (D3)

### 1-(2,2,2-trifluoroethyl)-4-piperidinecarboxylic acid

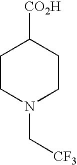

Solution of ethyl 1-(2,2,2-trifluoroethyl)-4-piperidinecarboxylate (0.632 g, 2.64 mmol) in EtOH (11.0 ml) was stirred at room temperature. 2M NaOH (3.0 ml, 6.00 mmol) was added and the solution stirred for 1½ hours at room temperature. Mixture was neutralised using 2M HCl and organics extracted into EtOAc (×2). Combined organics were dried over MgSO₄, filtered and concentrated under reduced pressure to give a pale yellow coloured oil. Residue was used without further purification.

### Description 4 (D4)

### 5-Ethenyl-2-pyridinamine

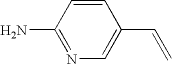

2-amino-5-bromo-pyridine (2.00 g, 11.56 mmol) was dissolved in a 1:1 mix of toluene (58 ml) and EtOH (58 ml). Vinyl boronic anhydride pyridine complex (VBAP) (4.17 g, 87.34 mmol) and K₂CO₃ (12.78 g, 92.5 mmol) were added to the solution. Argon was bubbled through the solution for 30 min. Pd(PPh₃)₄ (0.67 g, 0.58 mmol) was added and the solution was heated to 80° C. for a total of 3 hours. The mixture was allowed to cool to room temperature, then it was diluted with EtOAc and water. The organics were washed with further water. The organics were dried over MgSO₄, filtered and concentrated to give an orange oil. The oil was dissolved in the minimal amount of IPA which upon cooling a cream coloured precipitate formed. The solid was filtered off, the filtrate was concentrated and chromatographed [SiO₂, 75-100% EtOAc in Hexane] to give the title compound (526 mg).

LC/MS Rt=0.81 min, [M+H]⁺ 121

### Description 5 (D5)

### Methyl 6-(chloromethyl)-2-pyridinecarboxylate

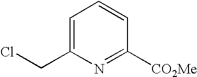

Thionyl chloride (1.785 g, 15 mmol) was added to a solution of methyl 6-(hydroxymethyl)-2-pyridinecarboxylate (2.088 g, 12.5 mmol) in dichloromethane (50 ml) and left at room temperature for one hour. The resulting solution was washed with 1M potassium carbonate solution, dried (magnesium sulphate) and evaporated to give the title compound as a colourless oil (2.32 g).

LC/MS: Rt=2.01 min, [M+H]⁺ 186.1, 188.1

### Description 6 (D6)

### Ethyl 6-{[5-chloro-2-(methyloxy)phenyl]methyl}-2-pyridinecarboxylate

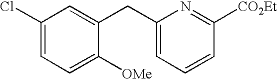

A mixture of 5-chloro-2-methoxyphenylboronic acid (2.33 g, 12.5 mmol), methyl 6-(chloromethyl)-2-pyridinecarboxylate (2.32 g, 12.5 mmol), potassium carbonate (6.9 g, 50 mmol) and tetrakis(triphenylphosphine)palladium(0) (724 mg, 0.625 mmol) in 1:1 ethanol/toluene (100 ml) was stirred and heated at 90° C. under argon for 3 hours. The mixture was cooled, diluted with water (200 ml) and ether (50 ml) and the organic phase dried (magnesium sulphate), evaporated and purified by flash chromatography on a Biotage column eluting with 1:4 ethyl acetate/hexane. The title compound was isolated as a colourless oil (1.8 g).

LC/MS: Rt=3.19, [M+H]⁺ 306.1, 308.1

### Description 7 (D7)

### Ethyl 5-{[5-chloro-2-(methyloxy)phenyl]methyl}-2-furancarboxylate

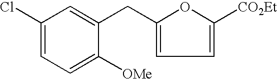

Prepared in a similar manner to above but using ethyl 5-chloromethyl-2-furancarboxylate instead of methyl 6-(chloromethyl)-2-pyridinecarboxylate.

LC/MS: Rt=3.43 min, [M+H]⁺ 295.2, 297.2

### Description 8 (D8)

### Ethyl 6-[(5-chloro-2-hydroxyphenyl)methyl]-2-pyridinecarboxylate

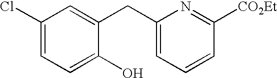

Boron tribromide (4.52 g, 18 mmol) was added carefully to a solution of ethyl 6-{[5-chloro-2-(methyloxy)phenyl]methyl}-2-pyridinecarboxylate (1.8 g, 5.89 mmol) in dichloromethane (25 ml) and left at room temperature for 4 hours. A further (4.52 g, 18 mmol) of boron tribromide were added and after 16 hours the solution was poured onto ice. Ethyl acetate (100 ml) was added and the organic phase was dried (magnesium sulphate) and evaporated to give a pale yellow foam (1.66 g) which was dissolved in ethanol (40 ml) and sulphuric acid (3 ml) and refluxed for 6 hours. The resulting solution was cooled, evaporated, dissolved in ethyl acetate/water (60 ml of each), basified with potassium carbonate and the organic phase dried, (magnesium sulphate) and evaporated to give a white solid which was triturated with 2:1 hexane/ether to give the title compound as a white solid (1.09 g).

LC/MS: Rt=3.07 min, [M+H]⁺ 292.2, 294.2

### Description 9 (D9)

### Ethyl 5-[(5-chloro-2-hydroxyphenyl)methyl]-2-furancarboxylate

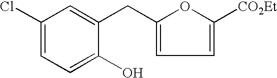

Prepared in a similar manner to above but using ethyl 5-{[5-chloro-2-(methyloxy)phenyl]methyl}-2-furancarboxylate instead of ethyl 6-{[5-chloro-2-(methyloxy)phenyl]methyl}-2-pyridinecarboxylate.

LC/MS: Rt=3.03 min, [M+H]⁺ 281.2, 283.2

### Description 10 (D10)

### Methyl 2-hydroxy-5-(methylsulfonyl)benzoate

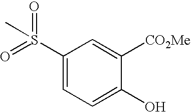

Boron tribromide (30.12 g, 120 mmol) was added carefully to an ice-cooled, stirred solution of methyl 2-(methyloxy)-5-(methylsulfonyl)benzoate (9.76 g, 40 mmol) in dichloromethane (200 ml) producing a gummy precipitate. Stirred for 30 minutes and the solution was poured onto ice and the gummy precipitate dissolved in ethyl acetate/water (200 ml of each). The ethyl acetate and the dichloromethane solutions were dried (magnesium sulphate) combined and evaporated. The residue was dissolved in methanol (150 ml) and sulphuric acid (10 ml) and refluxed under argon for 20 hours. The resulting solution was cooled, evaporated, dissolved in ethyl acetate/water (200 ml of each) and the organic phase washed with saturated sodium bicarbonate, dried (magnesium sulphate) and evaporated to give the title compound as a white solid (7.9 g).

LC/MS: Rt=2.11 min, [M+H]⁺ 231.2

### Description 11 (D11)

### Ethyl 6-[(5-chloro-2-hydroxy-3-iodophenyl)methyl]-2-pyridinecarboxylate

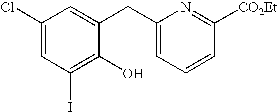

N-Iodosuccinimide (900 mg, 4 mmol) was added to a stirred solution of ethyl 6-[(5-chloro-2-hydroxyphenyl)methyl]-2-pyridinecarboxylate in DMF (6 ml) and stirred for 18 hours. The resulting solution was diluted with water (50 ml) and ethyl acetate (50 ml) and the organic phase washed with 5% sodium thiosulphate solution (50 ml) and water (3×25 ml) then dried (magnesium sulphate), evaporated and purified by flash chromatography on a Biotage column eluting with 1:4 ethyl acetate/hexane. The title compound was isolated as a white solid (1.34 g).

LC/MS: Rt=3.70 min, [M+H]⁺ 418.0, 420.0

The following compounds were prepared in a similar manner to above by reaction of N-iodosuccinimide with the appropriate phenol.

| Description | Structure | Name | LC/MS data |
|---|---|---|---|
| 12 (D12) | 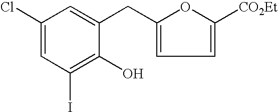 | Ethyl 5-[(5-chloro-2- hydroxy-3- iodophenyl)methyl]-2- furancarboxylate | Rt = 3.37 min, [M + H]⁺ 407.0 |
| 13 (D13) | 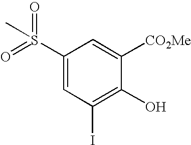 | Methyl 2-hydroxy-3- iodo-5- (methylsulfonyl)benzoate | Rt = 2.65 min, [M + H]⁺ 357.0 |

### Description 14 (D14)

### Methyl 3-bromo-5-chloro-2-hydroxybenzoate

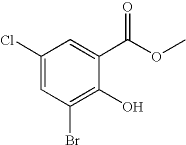

NBS (47.76 g) added to a stirred solution of methyl-5-chloro-salicylate (50.02 g) in DMF (500 ml) at rt, stirred overnight. Solid collected by filtration. Further solid formed when product was dried (air-flow) on sinter. Solid was collected. Filtrate was diluted with Et₂O and washed with H₂O. Et₂O layer was dried (Na₂SO₄), filtered and conc to low volume—solid collected and washed with cold Et₂O to give the title compound (68.05 g, 96%) Rt=3.42 mins [M−H]⁻ 265, 267

### Description 15 (D15)

### 3-bromo-5-chloro-2-hydroxybenzaldehyde

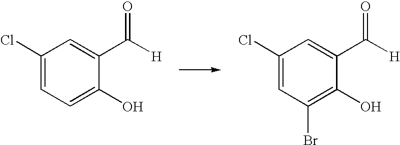

A solution of 5-chloro-2-hydroxybenzaldehyde (5.00 g, 31.93 mmol) and N-bromosuccinimide (5.68 g, 31.93 mmol) in dry DMF (64 ml) was stirred at room temperature under an atmosphere of argon for 72 hours (over the weekend). The reaction was monitored by LC-MS. The reaction mixture was diluted with EtOAc and washed with water (×3). Brine was added to encourage separation. The organics were dried over magnesium sulfate, filtered and concentrated under reduced pressure to give an orange solid, 3-bromo-5-chloro-2-hydroxybenzaldehyde (7.42 g, 99%)

Rt=2.92 min, [M−H]⁻ 233, 235

### Description 16 (D16)

### Methyl 5-chloro-2-hydroxy-3-iodobenzoate

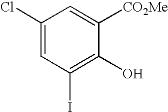

To a solution of methyl-5-chlorosalicylate (50 g) in N,N-dimethylformamide (400 ml) at room temperature was added N-iodosuccinimide (72.6 g) and the solution was stirred under argon over the weekend. The N,N-dimethylformamide was mostly removed (approximately 350 ml was evaporated) and the residue was filtered washing the white solid with N,N-dimethylformamide (100 ml). The solid was dried in the vacuum oven at 55° C. to afford the title compound 55.3 g.

LC/MS Rt=3.39 min. Molecular ion observed [M+H]⁺ 313, consistent with molecular formula C8H6³⁵CIIO₃

The filtrate was evaporated to a low volume of N,N-dimethylformamide which afforded more solid and the mixture was refrigerated overnight. The solid was filtered off and washed with cold N,N-dimethylformamide. The solid was dried in the vacuum oven at 55° C. to afford the title compound 27.1 g.

LC/MS Rt=3.39 min. Molecular ion observed [M+H]⁺ 313, consistent with molecular formula C8H6³⁵CIIO₃

### Description 17 (D17)

### 5-Chloro-2-hydroxy-3-iodobenzaldehyde

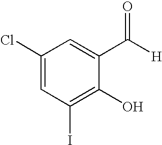

N-iodosuccinimide (7.18 g, 31.9 mmol) was slowly added to a solution of 5-Chloro-2-hydroxybenzaldehyde (5 g, 31.9 mmol) in DMF (25 ml). The reaction mixture was stirred at room temperature for 6 hours, more N-iodosuccinimide (1.8 g) was added and the reaction stirred for other 24 hours. The mixture was diluted with ethyl acetate (100 ml), washed with 0.1N HCl (40 ml), water (30 ml), 10% sodium thiosulphate solution (50 ml) and brine (30 ml). The organic phase was dried (MgSO₄) and evaporated to give the title compound as a yellow solid (8.82 g).

LC/MS Rt=3.84 min, [M−H]⁻ 280.9, 282.9

The following compound was prepared in a similar manner to 5-Chloro-2-hydroxy-3-iodobenzaldehyde using the appropriate intermediates:

| Description | Structure | Name | LC/MS data |
|---|---|---|---|
| 18 (D18) | 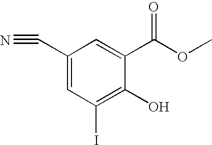 | Methyl 5-cyano-2- hydroxy-3- iodobenzoate | Rt =3.02 min, [M − H]⁻ 304 |

### Description 19 (D19)

### 1,1-Dimethylethyl 2-[(5-chloro-2-hydroxy-3-iodophenyl)methyl] hydrazinecarboxylate

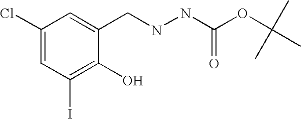

5-Chloro-2-hydroxy-3-iodobenzaldehyde (8.82 g, 31.2 mmol) was dissolved in dry DCM (90 ml), tert-butyl carbazate (4.53 g, 34 mmol) was added followed by acetic acid (1.87 ml, 32 mmol). The mixture was stirred under argon at room temperature for 40 minutes, cooled to 0° C., and sodium triacetoxyborohydride was slowly added. The suspension was warmed to room temperature, stirred for 2 hours, more sodium triacetoxyborohydride (5 g) was added and the reaction left for a further 72 hours. The mixture was treated slowly with 2M HCl (25 ml) and extracted with DCM (100 ml×2). The combined extracts were dried (MgSO₄) and evaporated to give the title compound as pale yellow solid.

LC/MS Rt=3.38 min, [M−H]⁻ 397,399

### Description 20 (D20)

### 1,1-Dimethylethyl 2-[(3-bromo-5-chloro-2-hydroxyphenyl)methyl]hydrazinecarboxylate

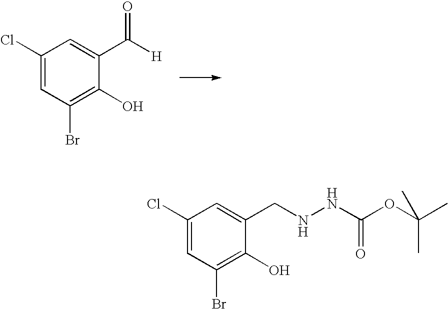

To a solution of 3-bromo-5-chloro-2-hydroxybenzaldehyde (7.42 g, 31.63 mmol) in dry DCM (90 ml), 1,1-dimethylethyl hydrazinecarboxylate (4.60 g, 34.80 mmol) and acetic acid (1.90 ml) were added. The reaction solution was stirred at room temperature under an atmosphere of argon for 40 minutes. After this time, the reaction solution was cooled to 0° C. and sodium triacetoxyborohydride (20.0 g, 94.90 mmol) was added. The reaction solution was stirred at room temperature for 2 hours. The reaction was monitored by LC-MS. The reaction mixture was diluted by the slow addition of hydrochloric acid (20 ml, 2M aq. soln). The organics were extracted using DCM (×2). Brine was added to encourage separation. The combined organics were dried over magnesium sulfate, filtered and concentrated under reduced pressure. The resulting residue was washed with 1:1 Hexane: Diethyl ether (50 ml) and filtered to give 1,1-dimethylethyl 2-[(3-bromo-5-chloro-2-hydroxyphenyl)methyl]hydrazinecarboxylate (11.0 g, 99%)

LC/MS Rt=3.13 min, [M−H]⁻ 351, 353

### Description 21 (D21)

### 4-Chloro-2-(hydrazinomethyl)-6-iodophenol

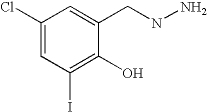

1,1-Dimethylethyl 2-[(5-chloro-2-hydroxy-3-iodophenyl)methyl] hydrazinecarboxylate (14.6 g, 36.6 mmol) was dissolved in THF (200 ml), HCl (5M aqueous solution) (29 ml, 146 mmol) was added and the solution was stirred, under argon, at 80° C. for 2 hours. The mixture was cooled and the solvent evaporated; the residue was diluted with water, basified with K₂CO₃ and extracted with ethyl acetate (500 ml). The organic phase was dried (MgSO₄) and evaporated. The residue was triturated with DCM to give the title compound as solid (3.5 g).

LC/MS Rt=1.57 min, [M+H]⁺ 299, 301

### Description 22 (D22)

### 2-Bromo-4-chloro-6-(hydrazinomethyl)phenol hydrochloride

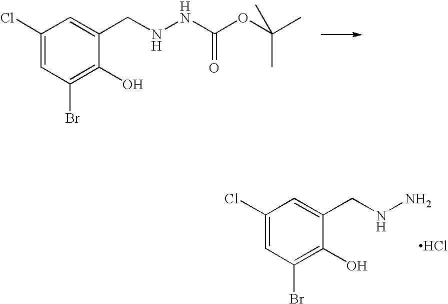

A solution of 1,1-dimethylethyl 2-[(3-bromo-5-chloro-2-hydroxyphenyl)methyl]-hydrazinecarboxylate (7.30 g, 20.8 mmol) and hydrochloric acid (17 ml, 5 M aq. soln) in THF (52 ml) was stirred at 80° C. under an atmosphere of argon for 4 hours. The reaction was monitored by LC-MS. The reaction solution was concentrated under reduced pressure to give a cream solid, 2-bromo-4-chloro-6-(hydrazinomethyl)phenol hydrochloride (6.15 g, 100%)

LC/MS Rt=1.24 min, [M−H]⁻ 250, 252

### Description 23 (D23)

### Ethyl 1-[(3-bromo-5-chloro-2-hydroxyphenyl)methyl]-5-methyl-1H-pyrazole-3-carboxylate

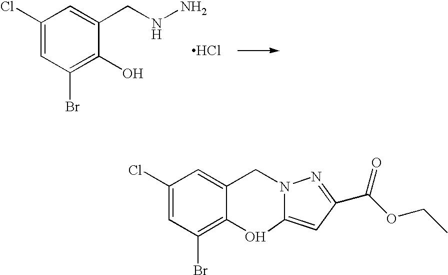

To a solution of 2-bromo-4-chloro-6-(hydrazinomethyl)phenol hydrochloride (3.72 g, 12.95 mmol) in acetic acid (25 ml) and EtOH (10 ml) at 70° C. under an atmosphere of argon, ethyl 2,4-dioxopentanoate (2 ml, 14.24 mmol) was added dropwise. An exotherm of 3° C. was observed during the addition. After the addition, the reaction solution was stirred at 70° C. for 30 minutes. The reaction was monitored by LC-MS. The reaction was allowed to cool to room temperature. Precipitation occurred and filtered. Solid washed with acetic acid and dried under high vacuum, ethyl 1-[(3-bromo-5-chloro-2-hydroxyphenyl)methyl]-5-methyl-1H-pyrazole-3-carboxylate. The filtrate was diluted with EtOAc and washed with water (×3). The organics were dried over magnesium sulfate, filtered and concentrated under reduced pressure to give ethyl 1-[(3-bromo-5-chloro-2-hydroxyphenyl)methyl]-5-methyl-1H-pyrazole-3-carboxylate.

LC/MS Rt=3.09 min, [M+H]⁺ 375, 377

### Description 24 (D24)

### Ethyl 1-[(5-chloro-2-hydroxy-3-iodophenyl)methyl]-5-methyl-1H-pyrazole-3-carboxylate

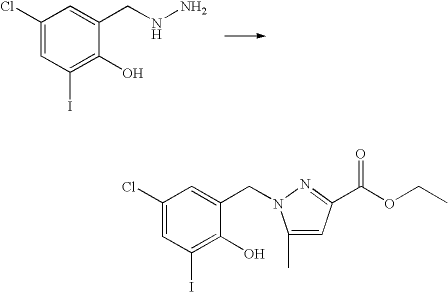

4-Chloro-2-(hydrazinomethyl)-6-iodophenol (3.5 g, 11.7 mmol) was dissolved in acetic acid (25 ml) and ethanol (4 ml) was added to help the dissolution. To this solution ethyl 2,4-dioxopentanoate (1.82 ml, 12.9 mmol) was slowly added; a white solid precipitated, the solid was filtered and triturated with dichloromethane/hexane mixture to give 1.77 g of the title compound as white solid. The mother liquor was concentrated and purified on the SP4 eluting with ethyl acetate 0-30% in hexane giving more product. Obtained in total 2.65 g of the title compound.

LC/MS Rt=3.37 min, [M+H]⁺ 421, 423, [M−H]⁻ 419, 420.9

### Description 25 (D25)

### Methyl 5-chloro-2-(2,4-dichlorophenyl)-1-benzofuran-7-carboxylate

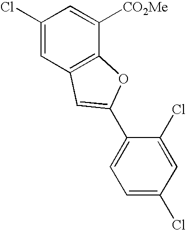

Solution of 2,4-dichloroiodobenzene (0.500 g, 1.85 mmol) in dry DMF (7.5 ml) was stirred at room temperature under an atmosphere of argon. (Ph₃P)₂PdCl₂ (0.133 g, 0.19 mmol), Et₃N (0.514 ml, 3.7 mmol), CuI (0.036 g, 0.19 mmol) and trimethylsilylacetylene (0.287 ml, 2.04 mmol) were added. The mixture was stirred for 40 minutes. After this time, TBAF (2.04 ml, 2.04 mmol, 1M in THF) and methyl 5-chloro-2-hydroxy-3-iodobenzoate (0.861 g, 2.77 mmol) and the mixture stirred for a further 2 hours at room temperature. After this time, the mixture was diluted with EtOAc and the organics washed with water. The combined organics were dried over MgSO₄, filtered and concentrated under reduced pressure to give a brown oil. The residue was chromatographed [SiO₂, Hexane/EtOAc, 0-10%) to give the title compound (0.523 g).

LC/MS Rt=4.34 min [M+H]⁺ 353, 357

### Description 26 (D26)

### Methyl 5-chloro-2-phenyl-1-benzofuran-7-carboxylate

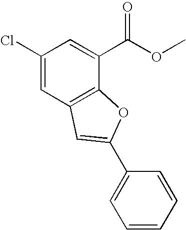

Ethynylbenzene (27.74 ml, 253 mmol) was added to a mixture of methyl 3-bromo-5-chloro-2-hydroxybenzoate (33.66 g, 126 mmol), CuI (2.41 g, 12.6 mmol), Pd (PPh₃)₂Cl₂ (8.84 g, 12.6 mmol) and TEA (35.2 ml, 253 mmol) in DMF (125 ml) under argon. The reaction mixture was stirred at room temperature for 1 hour, heated at 75° C. for 1 hour, cooled, diluted with water (400 ml) and extracted with diethyl ether (3×300 ml). The combined extracts were washed with water (3×150 ml), dried (MgSO₄) and evaporated. The residue was purified on the Biotage 75 using 5% of ethyl acetate in hexane, the fractions containing the product (impurity present) were combined and partially evaporated to give a solid that was filtered off to give the clean title compound as a pale yellow solid.

LC/MS Rt=3.84 min, [M+H]⁺ 287.1, 289.1

The following compounds were prepared from a bromo or iodophenol and the appropriate acetylene using the above method.

| Description | Structure | Name | LC/MS data |
|---|---|---|---|
| 27 (D27) | 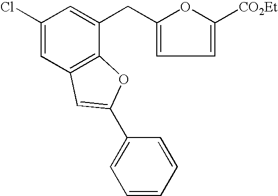 | Ethyl 5-[(5-chloro-2- phenyl-1-benzofuran-7- yl)methyl]-2- furancarboxylate | Rt = 4.01 min, [M + H]⁺ 381.1 |
| 28 (D28) | 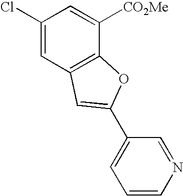 | Methyl 5-chloro-2-(3- pyridinyl)-1-benzofuran- 7-carboxylate | Rt = 3.03 min, [M + H]⁺ 288.2, 290.1 |
| 29 (D29) | 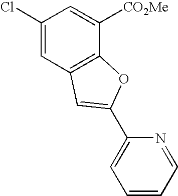 | Methyl 5-chloro-2-(2- pyridinyl)-1-benzofuran- 7-carboxylate | Rt = 3.25 min, [M + H]⁺ 288.2, 290.1 |
| 30 (D30) | 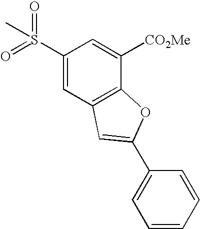 | Methyl 5- (methylsulfonyl)-2- phenyl-1-benzofuran-7- carboxylate | Rt = 3.04 min, [M + H]⁺ 331.1 |
| 31 (D31) | 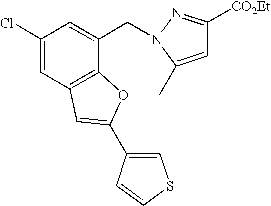 | Ethyl 1-{[5-chloro-2-(3- thienyl)-1-benzofuran-7- yl]methyl}-5-methyl-1H- pyrazole-3-carboxylate | Rt = 3.60 min, [M + H]⁺ 401.1, 403.1 |
| 32 (D32) | 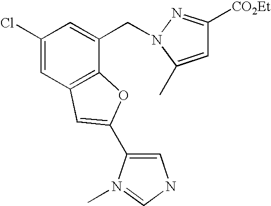 | Ethyl 1-{[5-chloro-2-(1- methyl-1H-imidazol-5-yl)- 1-benzofuran-7- yl]methyl}-5-methyl-1H- pyrazole-3-carboxylate | Rt = 2.23 min, [M + H]⁺ 399.2, 401.2 |
| 33 (D33) | 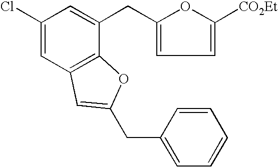 | Ethyl 5-{[5-chloro-2- (phenylmethyl)-1- benzofuran-7-yl]methyl}- 2-furancarboxylate | Rt = 3.93 min, [M4-H]⁺ 395.1 |

### Description 34 (D34)

### Methyl 5-cyano-2-phenyl-1-benzofuran-7-carboxylate

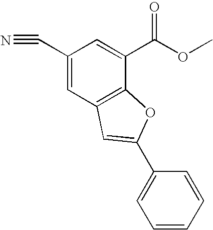

Phenylacetylene (5.6 ml, 51.05 mmol) was added to methyl 5-cyano-2-hydroxy-3-iodobenzoate (7.8 g, 25.7 mmol), CuI (0.49 g, 2.57 mmol), Pd(PPh₃)₂Cl₂ (1.8 g, 2.56 mmol) and TEA (7.15 ml, 51.5 mmol) in DMF (60 ml), under nitrogen. The reaction mixture was stirred for 18 hours at room temperature. LC/MS consistent with product and PPh₃. H₂O (200 ml) was added and the solution extracted with ethyl acetate (100 ml×3). Some of the product precipitated out, it was then filtered off. The organic layer was washed with H₂O (100 ml×2), dried (MgSO₄) and evaporated to give a black oil. The residue was chromatographed on the SP4 using 10-30% EtOAc in hexane. The title compound was obtained as an orange coloured solid (6.2 g)

LC/MS Rt=3.37 min, [M+H]⁺ 278.2.

### Description 35 (D35)

### Methyl 5-chloro-2-(2,4-difluorophenyl)-1-benzofuran-7-carboxylate

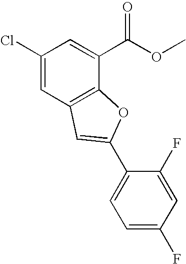

To a solution of 2,4-difluoro-1-iodobenzene (7.69 g, Aldrich) in N,N dimethylformamide (130 ml) and triethylamine (8.9 ml) was added dichlorobis(triphenylphosphine)-palladium(II) (2.25 g), copper(I)iodide (608 mg) and trimethylsilylacetylene (5 ml). The solution was stirred at room temperature under argon for forty minutes and then treated with a solution of tetrabutylammonium fluoride (1.0M in tetrahydrofuran, 35 ml) followed by methyl 5-chloro-2-hydroxy-3-iodobenzoate (15 g), added portionwise. After three hours, the N,N-dimethylformamide was evaporated and the residue dissolved in ethyl acetate (400 ml). The ethyl acetate layer was washed with water (2×400 ml) and then dried (MgSO₄) and evaporated to a dark brown oily solid. The residue was triturated with diethyl ether and the diethyl ether was decanted off. This was repeated twice and then the combined diethyl ether solutions were evaporated to afford a brown oil. The oil was dissolved in dichloromethane and applied to a Biotage Si 75 column. The column was eluted with hexane (2 L) followed by 5% ethyl acetate/hexane (6 L). All fractions containing clean product by t.l.c. were taken evaporated and dried to a yellow solid. The solid was stirred in hexane for one hour then filtered off and dried at 40° C. under vacuum to afford the title compound (1.88 g). The filtrate was evaporated to give an impure second crop of the title compound (1.55 g)

LC/MS Rt=3.87 min. Molecular ion observed [M+H]⁺ 323, consistent with molecular formula C₁₆H₉O₃³⁵ClF₂

### Description 36 (D36)

### 5-Chloro-2-(4-fluorophenyl)-1-benzofuran-7-carboxylic acid

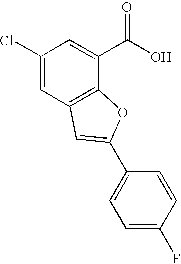

Methyl 5-chloro-3-formyl-2-hydroxybenzoate (800 mg, 3.73 mmol), ethyl bromo(4-fluorophenyl)acetate (1.07 g, 4.1 mmol) and K₂CO₃ (2.21 g, 16 mmol) were heated in DMF (10 ml), under argon, for 2½ hour. Cooled, neutralised with 2M HCl and extracted with ethyl acetate (×2). The combined extracts were washed with water, dried and evaporated to give a yellow oil.

The oil was dissolved in ethanol (16 ml) and KOH (1.19 g, 21.2 mmol) added. The reaction mixture was refluxed, under argon, for 3 hours; it was then cooled and the solvent was evaporated. The residue was treated with water, acidified with 2M HCl and extracted with EtOAc (×3), the combined extracts were dried (MgSO₄) and evaporated to give an orange solid.

The solid was dissolved in xylene (20 ml) and p-toluene sulfonic acid (˜120 mg) added. The mixture was refluxed for 1 hour, it was then cooled and the solvent evaporated. The residue was diluted with ethyl acetate and washed with water, the organic phase was dried (MgSO₄) and evaporated to give a dark solid. Trituration with diethyl ether gave the title compound as a pale brown solid (660 mg).

LC/MS Rt=3.25 min, [M+H]⁺ 289, 291

### Description 37 (D37)

### Ethyl 1-{[5-chloro-2-(4-chloro-2-fluorophenyl)-1-benzofuran-7-yl]methyl}-5-methyl-1H-pyrazole-3-carboxylate

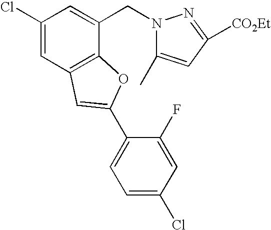

Solution of ethyl 1-[(5-chloro-2-hydroxy-3-iodophenyl)methyl]-5-methyl-1H-pyrazole-3-carboxylate (0.97 g, 2.31 mmol) in dry DMF (9.2 ml) was stirred at room temperature under an atmosphere of argon. Trimethylsilylacetylene (0.391 ml, 2.77 mmol), (Ph₃P)₂PdCl₂ (0.162 g, 0.23 mmol), CuI (0.045 g, 0.23 mmol) and Et₃N (0.642 ml, 4.62 mmol) were added to the solution. The solution was stirred at room temperature for 1 hour. After this time, TBAF (1M in THF, 3.47 ml, 3.47 mmol) and 4-chloro-2-fluoro-iodobenzene (0.887 g, 0.448 ml, 3.47 mmol) were added and the mixture stirred at room temperature for 1 hour. After this time, further 4-chloro-2-fluoro-iodobenzene (0.2 ml) was added and the mixture heated to 70° C. for 2½ hours. After this time, the solution was allowed to cool to room temperature. The mixture was then diluted with EtOAc and the organics were washed with water (×3). The concentrated organics were dried over MgSO₄, filtered and concentrated under reduced pressure to give a brown oily solid. The residue was chromatographed [SiO₂, Hexane/EtOAc, 0-35%]. The obtained compound was then further purified using MDAP to give the title compound (0.149 g)

LC/MS Rt=4.11 min [M+H]⁺ 447

### Description 38 (D38)

### Ethyl 1-{[5-chloro-2-(2-chlorophenyl)-1-benzofuran-7-yl]methyl}-5-methyl-1H-pyrazole-3-carboxylate

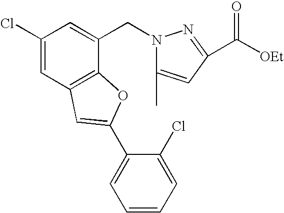

Solution of ethyl 1-[(5-chloro-2-hydroxy-3-iodophenyl)methyl]-5-methyl-1H-pyrazole-3-carboxylate (1.00 g, 2.38 mmol) in dry DMF (5.0 ml) was stirred at room temperature under an atmosphere of argon. 2-chlorophenyl acetylene (0.485 g, 3.57 mmol), (Ph₃P)₂PdCl₂ (0.168 g, 0.24 mmol), CuI (0.045 g, 0.24 mmol) and Et₃N (0.728 ml, 4.76 mmol) were added and the solution was stirred at room temperature for 18 hours. After this time, solution was heated to 80° C. for 3 hours. The solution was then allowed to cool to room temperature. The solution was diluted with EtOAc and the organics washed with water (×2). Organics were dried over MgSO₄, filtered and concentrated under reduced pressure to give a dark coloured oily solid. The residue was chromatographed [SiO₂, Hexane/EtOAc 0-25%] to give the title compound (0.550 g)

LC/MS Rt=3.74 min [M+H]⁺ 429, 432.

### Description 39 (D39)

### Ethyl 1-[(5-chloro-2-phenyl-1-benzofuran-7-yl)methyl]-5-methyl-1H-pyrazole-3-carboxylate

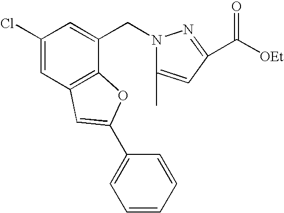

Solution of ethyl 1-[(5-chloro-2-hydroxy-3-iodophenyl)methyl]-5-methyl-1H-pyrazole-3-carboxylate (7.00 g, 16.67 mmol) in dry DMF (65.0 ml) was stirred at room temperature under an atmosphere of argon. Phenylacetylene (2.743 ml, 25.00 mmol), (Ph₃P)₂PdCl₂ (1.170 g, 1.67 mmol), CuI (0.318 g, 1.67 mmol) and Et₃N (4.638 ml, 33.34 mmol) were added and the solution was stirred at room temperature for 19 hours. After this time, solution was heated to 90° C. for 1 hour. The solution was then allowed to cool to room temperature. The solution was diluted with EtOAc and the organics washed with water (×2). Organics were dried over MgSO₄, filtered and concentrated under reduced pressure to give a black coloured oily solid. The residue was chromatographed [SiO₂, Hexane/EtOAc 15-25%] to give the title compound (4.08 g)

LC/MS Rt=3.72 min [M+H]⁺ 395, 397.

### Description 40 (D40)

### Ethyl 1-[5-chloro-2-(5-fluoro-2-pyridinyl)-1-benzofuran-7-yl]methyl-5-methyl-1H-pyrazole-3-carboxylate

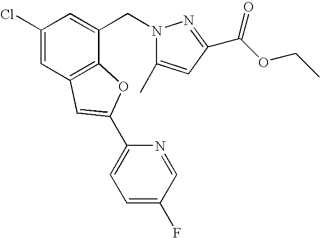

To a solution of 2-bromo-5-fluoropyridine (322 mg, Aldrich) in N,N dimethylformamide (6 ml) and triethylamine (0.510 ml) was added dichlorobis(triphenylphosphine)-palladium(II) (128 mg), copper(I)iodide (35 mg) and trimethylsilylacetylene (0.284 ml). The solution was stirred at room temperature under argon for ninety minutes and then treated with a second portion of trimethylsilylacetylene (0.050 ml). After a total of three and a half hours, a solution of tetrabutylammonium fluoride (1.0M in tetrahydrofuran, 2 ml) followed by ethyl 1-[(5-chloro-2-hydroxy-3-iodophenyl)methyl]-5-methyl-1H-pyrazole-3-carboxylate (1 g) was added and the mixture stirred at room temperature under argon for one hour then at 70° C. for two hours. The N,N-dimethylformamide was partially evaporated and the residue dissolved in ethyl acetate (40 ml). The ethyl acetate layer was washed with water (3×40 ml) and then dried (MgSO₄) and evaporated to a brown oil. The oil was dissolved in dichloromethane and applied to a Biotage Si 40+M column (pre-wetted with hexane) and eluted with 20% ethyl acetate/hexane (500 ml) and 30% ethyl acetate/hexane (500 ml). Fractions were evaporated and dried to afford the title compound as a yellow foam (287 mg).

LC/MS Rt=3.50 min. Molecular ion observed [M+H]⁺ 414, consistent with molecular formula C₂₁H₁₇N₃O₃³⁵ClF

The following compound was prepared in a similar manner using the appropriate aryl halide:

| Description | Structure | Name | LC/MS data |
|---|---|---|---|
| 41 (D41) | 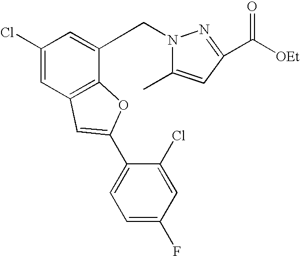 | ethyl 1-{[5-chloro-2- (2-chloro-4- fluorophenyl)-1- benzofuran-7- yl]methyl}-5-methyl- 1H-pyrazole-3- carboxylate | Rt = 3.80 min, [M + H]⁺ 447, 449, 451 |

### Description 42 (D42)

### Ethyl 1-{[5-chloro-2-(4-chlorophenyl)-1-benzofuran-7-yl]methyl}-5-methyl-1H-pyrazole-3-carboxylate

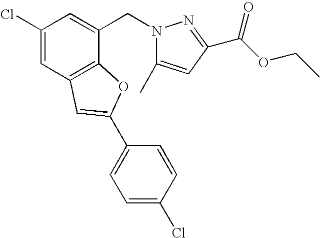

To a solution of ethyl 1-[(5-chloro-2-hydroxy-3-iodophenyl)methyl]-5-methyl-1H-pyrazole-3-carboxylate (1 g) in N,N dimethylformamide (10 ml) and triethylamine (0.662 ml) was added dichlorobis(triphenylphosphine)-palladium(II) (167 mg), copper(I)iodide (45 mg) and 1-chloro-4-ethynylbenzene (325 mg). The solution was stirred at room temperature under argon for forty five minutes and then treated with a second portion of 1-chloro-4-ethynylbenzene (162 mg). After stirring for a further one hour, the solution was refrigerated overnight. The reaction mixture was diluted with ethyl acetate (100 ml) and washed with water (3×50 ml) and then dried (MgSO₄) and evaporated to a brown solid. The solid was dissolved in dichloromethane and applied to a Biotage Si 40+M column (pre-wetted with hexane) and eluted with 10% ethyl acetate/hexane (500 ml) and 20% ethyl acetate/hexane (1 L) taking 10 ml fractions. Fractions 30-70 were evaporated and dried to afford the title compound as a pale brown foam (890 mg).

LC/MS Rt=3.91 min. Molecular ion observed [M+H]⁺ 429, consistent with molecular formula C₂₂H₁₈N₂O₃³⁵Cl₂

### Description 43 (D43)

### Ethyl 1-{[5-chloro-2-(2-cyanophenyl)-1-benzofuran-7-yl]methyl}-5-methyl-1H-pyrazole-3-carboxylate

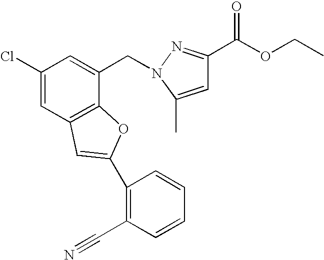

2-Iodobenzonitrile (340 mg, 1.48 mmol), Pd(PPh₃)₂Cl₂ (104 mg, 0.15 mmol), CuI (28 mg, 0.15 mmol), TEA (412 μl, 2.96 mmol) and trimethylsilyl acetylene (375 μl, 1.63 mmol) were stirred in DMF (6 ml), under argon, for 1 hour. Tetrabutylammonium fluoride (1.63 ml, 1M in THF, 1.63 mmol) and ethyl 1-[(5-chloro-2-hydroxy-3-iodophenyl)methyl]-5-methyl-1H-pyrazole-3-carboxylate (811 mg, 1.93 mmol) were added and the mixture was stirred at room temperature for 1½ hour then heated at 70° C. for 2 hours. The mixture was cooled, diluted with ethyl acetate (15 ml), washed with water; the organic phase was dried (MgSO₄) and evaporated. The residue was purified on the SP4 using 10-30% of EtOAc in hexane to afford the title compound as yellow oil (400 mg).

LC/MS Rt=3.62 min, [M+H]⁺ 420.1

The following compound was prepared in a similar manner to ethyl 1-{[5-chloro-2-(2-cyanophenyl)-1-benzofuran-7-yl]methyl}-5-methyl-1H-pyrazole-3-carboxylate from the appropriate intermediates:

| Description | Structure | Name | LC/MS data |
|---|---|---|---|
| 44 (D44) | 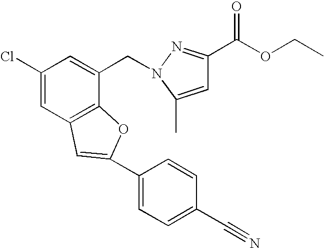 | Ethyl 1-{[5-chloro-2-(4- cyanophenyl)-1- benzofuran-7-yl]methyl}- 5-methyl-1H-pyrazole-3- carboxylate | Rt = 3.58 min, [M + H]⁺ 420.1, 422.1 |

### Description 45 (D45)

### Ethyl 6-[(5-chloro-2-phenyl-1-benzofuran-7-yl)methyl]-2-pyridinecarboxylate

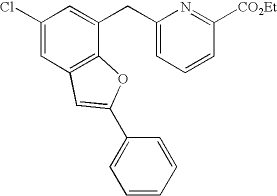

A mixture of ethyl 6-[(5-chloro-2-hydroxy-3-iodophenyl)methyl]-2-pyridinecarboxylate (2.46 g, 5.89 mmol), phenylacetylene (1.29 ml, 11.78 mmol), copper(I) iodide (112 mg, 0.59 mmol) triethylamine (1.64 ml, 11.79 mmol) and bis(triphenylphosphine)palladium(II) chloride (414 mg, 0.59 mmol) in DMF (10 ml) was stirred at room temperature for one hour then heated at 70° C. for 1.5 hours, left overnight at room temperature and heated for a further 4 hours at 70° C. The resulting mixture was diluted with ethyl acetate/water (250 ml of each) and the organic layer dried (magnesium sulphate) and evaporated. The residue was purified by repeated flash chromatography on a Biotage column eluting with 10-25% ethyl acetate/hexane, 0-20% ethyl acetate in hexane and finally 0-18% ethyl acetate/hexane to give the title compound as yellow solid (1.02 g). A further 1.33 g of less pure material was also obtained.

LC/MS: Rt=3.86 min, [M+H]⁺ 392.1, 394.1

### Description 46 (D46)

### Ethyl 1-{[5-chloro-2-(1,3-thiazol-2-yl)-1-benzofuran-7-yl]methyl}-5-methyl-1H-pyrazole-3-carboxylate

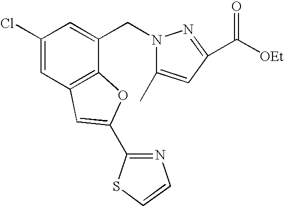

A solution of 2-bromothiazole (0.163 ml, 1.83 mmol) in dry DMF (6 ml) was stirred at room temperature. CuI (34 mg, 0.181 mmol), Pd(PPh₃)Cl₂ (127 mg, 0.181 mmol), Et₃N (0.504 ml, 3.62 mmol) and TMS-acetylene (0.281 ml, 1.99 mmol) were added. The solution was stirred at room temperature under argon for 1.5 hours. A further 1 equiv. of TMS acetylene was added. The solution was stirred at room temperature for a total of 18 hours. The solution was then heated to 40° C. under argon for a total of 4 hours. The solution was allowed to cool to room temperature. TBAF (3.8 ml) was added and the mixture left to stir at room temperature for a total of 18 hours and 15 minutes. The mixture was then heated to 40° C. under argon for 1 hour, then heated to 60° C. for 2 hours. The black solution was concentrated under reduced pressure. The resulting residue was diluted with EtOAc. The organics were washed with water (4×10 ml). Organics were dried over MgSO₄, filtered and concentrated to give a brown coloured oil. The oil was chromatographed [SiO₂, 25-50% EtOAc in Hexane] to give product. This sample contained an impurity, so sample was scratched in ether but no precipitate formed. The residue was dissolved in DMSO:MeOH (1:1), a precipitate formed which was collected following filtration. No improvement in purity was apparent. The solid was combined with the mother liquors and dissolved in DCM. The organics were washed with water (×2) to remove the DMSO. Organics were dried over MgSO₄, filtered and concentrated to give the title compound (impurity remaining). (79 mg)

LC/MS Rt=3.28 min, [M+H]⁺ 402, 404

Impurity LC/MS Rt=4.01 min [M+H]⁺ 657

### Description 47 (D47)

### [5-Chloro-2-(2,4-dichlorophenyl)-1-benzofuran-7-yl]methanol

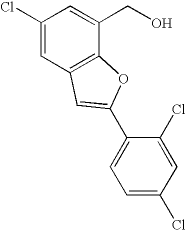

Solution of methyl 5-chloro-2-(2,4-dichlorophenyl)-1-benzofuran-7-carboxylate (2.369 g, 6.67 mmol) in dry THF (25.0 ml) was stirred at 0° C. under an atmosphere of argon. LiAlH₄ (1M in THF, 6.67 mmol, 6.67 ml) was added dropwise to the stirred solution. Solution was stirred at 0° C. for 3 hours. After this time, LC/MS was consistent with possible product. Solution was quenched by addition of water (dropwise initially). Organics were extracted into EtOAc and then washed with water (×3). Organics were dried over MgSO₄, filtered and concentrated under reduced pressure to give a light brown coloured solid. (2.08 g)

LC/MS Rt=3.81 min

### Description 48 (D48)

### [5-Chloro-2-(3-pyridinyl)-1-benzofuran-7-yl]methanol

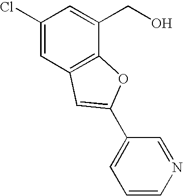

1M Lithium aluminum hydride in THF (2 ml, 2 mmol) was added to a stirred solution of methyl 5-chloro-2-(3-pyridinyl)-1-benzofuran-7-carboxylate (565 mg, 1.96 mmol) in dry TH F (15 ml) under argon and stirred for 30 minutes then cooled in ice and 2M sodium hydroxide (15 ml) added carefully followed by ethyl acetate (25 ml). The organic phase was dried (magnesium sulphate), evaporated and purified by flash chromatography on a Biotage column using a gradient elution from 1:1 to 9:1 ethyl acetate/hexane. The title compound was isolated as an off-white solid (326 mg).

LC/MS: Rt=2.29 min, [M+H]⁺ 260.1, 262.1

The following compounds were prepared in a similar manner by lithium aluminum hydride reduction of the appropriate ester.

| Description | Structure | Name | LC/MS data |
|---|---|---|---|
| 49 (D49) | 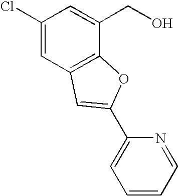 | [5-Chloro-2-(2- pyridinyl)-1-benzofuran- 7-yl]methanol | Rt = 2.59 min, [M +H]⁺ 260.2, 262.2 |
| 50 (D50) | 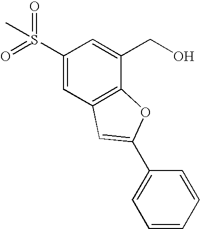 | [5-(Methylsulfonyl)-2- phenyl-1-benzofuran-7- yl]methanol | Rt = 2.62 min, |

### Description 51 (D51)

### (5-Chloro-2-phenyl-1-benzofuran-7-yl)methanol

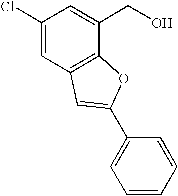

Methyl 5-chloro-2-phenyl-1-benzofuran-7-carboxylate (16.2 g, 56.5 mmol) was dissolved in tetrahydrofuran (250 ml) cooled to 0° C. and LiAlH₄ (24.6 ml, 2.3 M in THF, 56.5 mmol) was added dropwise under argon. The reaction mixture was warmed to room temperature and stirred for 1 hour, 2M HCl was slowly added and the solution was extracted with diethyl ether (2×250 ml). The combined organics were dried (MgSO₄) and evaporated to give an off white solid.

LC/MS Rt=3.26 min, [M+H]⁺ 259.2

The following compounds were prepared in a similar manner to (5-Chloro-2-phenyl-1-benzofuran-7-yl)methanol from the appropriate intermediates:

| De- | | | |
|---|---|---|---|
| scrip- | | | LC/MS |
| tion | Structure | Name | data |
| 52 (D52) | 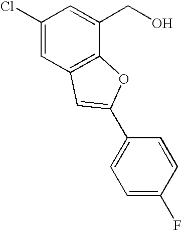 | [5-Chloro-2-(4- fluorophenyl)-1- benzofuran-7- yl]methanol | Rt = 3.32 min, [M + H]⁺ 277.1 |

### Description 53 (D53)

### 7-(Hydroxymethyl)-2-phenyl-1-benzofuran-5-carbonitrile

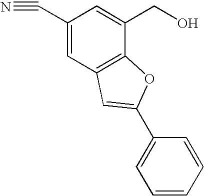

Methyl 5-cyano-2-phenyl-1-benzofuran-7-carboxylate (6.2 g, 22.00 mmol) was dissolved in THF (100 ml) and cooled to −10° C. LiAlH₄ (1M in Et₂O, 11.2 ml, 11.2 mmol) was added slowly under an atmosphere of argon. the mixture was kept cold for ½ hour then warmed to rt for ½ hour. Further LiAlH₄ (1M in Et₂O, 2.6 ml) was added and the mixture stirred for a further ½ hour at rt. H₂O (150 ml) and EtOAc (200 ml) were added and then the mixture was filtered through celite. The aqueous layer was extracted using EtOAc (100 ml). Organics were dried (MgSO₄) and evaporated to give a dark coloured solid. The solid was not very soluble in DCM, solid collected and the solution columned on SP4 15-35% EtOAc in Hexane. The insoluble solid was identified as the title compound (2 g). The column did not separate the title compound from impurities (2 g of crude material obtained).

LC/MS Rt=2.81 mins, [M+H]⁺ 216.2

### Description 54 (D54)

### [5-Chloro-2-(2,4-difluorophenyl)-1-benzofuran-7-yl]methanol

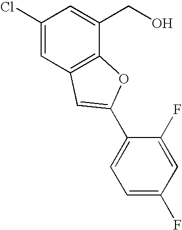

To solutions of methyl 5-chloro-2-(2,4-difluorophenyl)-1-benzofuran-7-carboxylate (1.88 g and 1.55 g) in dry tetrahydrofuran (25 ml and 20 ml) at 0° C. under argon was added dropwise solutions of lithium aluminum hydride (1.0M in tetrahydrofuran, 5.84 ml and 4.8 ml) and the solutions stirred at 0° C. for one hour. Both reaction mixtures were quenched with water and extracted with ethyl acetate. The organic layer from the first reaction was washed with water then dried (MgSO₄) and evaporated to afford the title compound as an off-white solid (1.69 g). The organic layer from the second reaction was washed with water then dried (MgSO₄) and evaporated to afford the title compound as a yellow solid (1.22 g).

LC/MS Rt=3.32 min. No molecular ion was observed.

### Description 55 (D55)

### 5-Chloro-7-(chloromethyl)-2-(2,4-difluorophenyl)-1-benzofuran

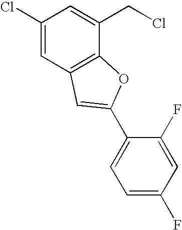

To suspensions of [5-chloro-2-(2,4-difluorophenyl)-1-benzofuran-7-yl]methanol (1.69 g and 1.22 g) in dry dichloromethane (20 ml and 15 ml) at room temperature under argon was added thionyl chloride (2.1 ml and 1.5 ml). After ten minutes solutions were obtained and the solutions were stirred overnight. Both reaction mixtures were evaporated to dryness and used in the next step without purification.

### Description 56 (D56)

### 5-Chloro-7-(chloromethyl)-2-(2,4-dichlorophenyl)-1-benzofuran

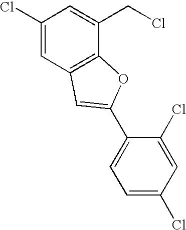

Mixture of [5-chloro-2-(2,4-dichlorophenyl)-1-benzofuran-7-yl]methanol (2.08 g, 6.36 mmol) in dry DCM (13.0 ml) was stirred at room temperature under an atmosphere of argon. SOCl₂ (2.63 ml, 31.8 mmol) was added and the mixture stirred for 3 hours (solid dissolved 10 minutes after addition of SOCl₂). After 3 hours at room temperature, solvent was removed under reduced pressure to give the title compound. The title compound was used directly, without further purification, in subsequent steps.

### Description 57 (D57)

### [5-Chloro-2-(3-pyridinyl)-1-benzofuran-7-yl]methyl methanesulfonate

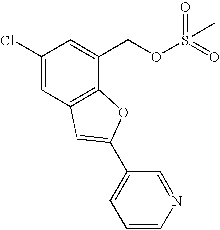

Methanesulphonyl chloride (149 mg, 1.3 mmol) was added to a stirred suspension of [5-chloro-2-(3-pyridinyl)-1-benzofuran-7-yl]methanol (325 mg, 1.25 mmol) and triethylamine (151 mg, 1.5 mmol) in dichloromethane (10 ml) producing a yellow solution. Stirred for 30 minutes then washed with water (20 ml), dried (magnesium sulphate) and evaporated to give the title compound as a yellow solid (420 mg).

LC/MS: Rt=2.75 min, [M+H]⁺ 338.1, 340.1

The following compound was prepared in a similar manner by reaction of methanesulphonyl chloride with the appropriate alcohol.

| Description | Structure | Name | LC/MS data |
|---|---|---|---|
| 58 (D58) | 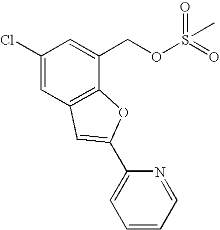 | [5-Chloro-2-(2- pyridinyl)-1- benzofuran-7- yl]methyl methanesulfonate | Rt = 3.00 min, [M + H]⁺ 338.1, 340.1 |

### Description 59 (D59)

### (5-Chloro-2-phenyl-1-benzofuran-7-yl)methyl methanesulfonate

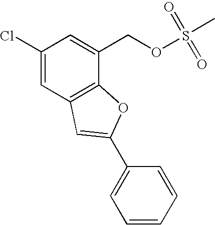

5-Chloro-2-phenyl-1-benzofuran-7-yl)methanol (16.1 g, 62.4 mmol) was treated with dichloromethane (200 ml), cooled to 0° C., triethylamine (10.4 ml, 75 mmol) and methane sulfonyl chloride (5.81 ml, 75 mmol) were added under argon. The mixture was stirred at 0° C. for 15 minutes and then stirred at room temperature for 1 hour. The solvent was evaporated; the residue was diluted with H₂O, acidified with 2M HCl and extracted with ethyl acetate (2×250 ml). The combined extracts were dried (MgSO₄) and evaporated to give the title compound as pale yellow solid (21.5 g).

LC/MS Rt=3.47 min

The following compounds were prepared in a similar manner to (5-Chloro-2-phenyl-1-benzofuran-7-yl)methyl methanesulfonate from the appropriate intermediates:

| Description | Structure | Name | LC/MS data |
|---|---|---|---|
| 60 (D60) | 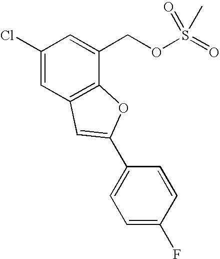 | [5-Chloro-2-(4- fluorophenyl)-1- benzofuran-7-yl]methyl methanesulfonate | Rt = 3.52 min |
| 61 (D61) | 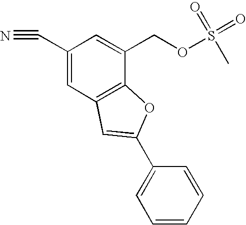 | (5-Cyano-2-phenyl-1- benzofuran-7-yl)methyl methanesulfonate | Rt = 3.17 min |

### Description 62 (D62)

### [5-(Methylsulfonyl)-2-phenyl-1-benzofuran-7-yl]methyl methanesulfonate

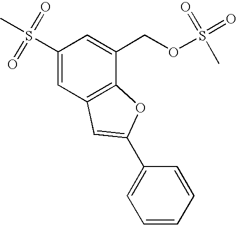

To a suspension of [5-(methylsulfonyl)-2-phenyl-1-benzofuran-7-yl]methanol (10 g) in dichloromethane (150 ml) was added triethylamine (6.89 ml) and the mixture stirred under argon until in solution. The solution was cooled to 0° C. and methanesulfonic anhydride (6.89 g) added portionwise and then stirred for thirty minutes at room temperature. The reaction mixture was diluted with dichloromethane (150 ml), washed with water (2×150 ml) dried (MgSO₄) and evaporated to afford the title compound (12.3 g).

LC/MS Rt=2.87 min. No molecular ion observed

### Description 63 (D63)

### Ethyl 1-{[5-chloro-2-(2,4-dichlorophenyl)-1-benzofuran-7-yl]methyl}-5-methyl-1H-pyrazole-3-carboxylate

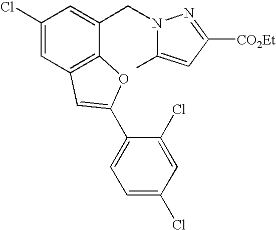

Solution of 5-chloro-7-(chloromethyl)-2-(2,4-dichlorophenyl)-1-benzofuran (6.36 mmol) in dry DMF (25.0 ml) was stirred at room temperature under an atmosphere of argon. Ethyl 3-methylpyrazole-5-carboxylate (1.078 g, 7.00 mmol), K₂CO₃ (0.966 g, 7.00 mmol) and NaI (1.421 g, 9.54 mmol) were added to the stirred solution. The solution was stirred at room temperature overnight. After this time only starting material was apparent by LC/MS. Reaction mixture was heated to 80° C. for 4 hours. Solution was diluted with EtOAc and washed with water (×3). Organics were dried over MgSO₄, filtered and concentrated under reduced pressure to give a brown coloured oil. The residue was chromatographed [SiO₂ Hexane/EtOAc, 10-20%) to give the title compound (0.738 g)

LC/MS Rt=4.04 min, [M+H]⁺ 465, 467.

### Description 64 (D64)

### Ethyl 1-{[5-chloro-2-(3-pyridinyl)-1-benzofuran-7-yl]methyl}-5-methyl-1H-pyrazole-3-carboxylate

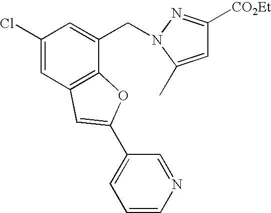

A mixture of [5-chloro-2-(3-pyridinyl)-1-benzofuran-7-yl]methyl methanesulfonate (420 mg, 1.24 mmol), ethyl 5-methyl-1H-pyrazole-3-carboxylate (200 mg, 1.3 mmol) and potassium carbonate (414 mg, 3 mmol) in DMF (10 ml) was stirred at room temperature for 17 hours then diluted with water (60 ml) and ethyl acetate (50 ml). The organic phase was washed with water (3×30 ml), dried (magnesium sulphate), evaporated and purified by flash chromatography on a Biotage column using a gradient elution from 1:1 to 3:1 ethyl acetate/hexane to give 180 mg of the title compound.

LC/MS Rt=3.07 min, [M+H]⁺ 396.1, 398.1

The following compounds were prepared in a similar manner by reaction of ethyl 5-methyl-1H-pyrazole-3-carboxylate with the appropriate methanesulphonate.

| Description | Structure | Name | LC/MS data |
|---|---|---|---|
| 65 (D65) | 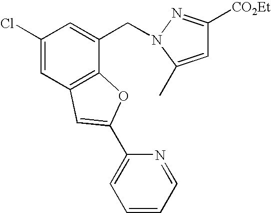 | Ethyl 1-{[5-chloro-2- (2-pyridinyl)-1- benzofuran-7- yl]methyl}-5-methyl- 1H-pyrazole-3- carboxylate | Rt = 3.28 min, [M + H]⁺ 396.1, 398.1 |
| 66 (D66) | 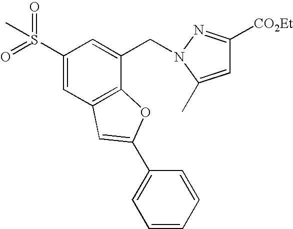 | Ethyl 5-methyl-1-{[5- (methylsulfonyl)-2- phenyl-1- benzofuran-7- yl]methyl}-1H- pyrazole-3- carboxylate | Rt = 3.10 min, [M + H]⁺ 439.2 |

### Description 67 (D67)

### Ethyl 1-[(5-chloro-2-phenyl-1-benzofuran-7-yl)methyl]-5-methyl-1H-pyrazole-3-carboxylate

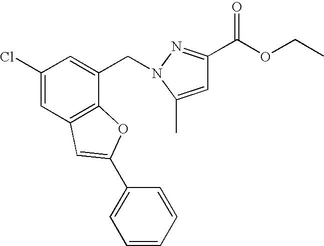

Ethyl 5-methyl-1H-pyrazole-3-carboxylate (10.3 g, 67 mmol) was added to (5-Chloro-2-phenyl-1-benzofuran-7-yl)methyl methanesulfonate (21.5 g, 64 mmol) and K₂CO₃(17.5 g, 127 mmol) in DMF (200 ml). The mixture was stirred under argon at room temperature for 64 hours, diluted with water (200 ml) and extracted with diethyl ether (2×250 ml). The combined extracts were dried (MgSO₄) and evaporated. The residue was purified on the SP4 using 20% of ethyl acetate in hexane, the solid obtained was triturated with a mixture of hexane/ethyl acetate to give the title compound as white solid (10.7 g).

LC/MS Rt=3.21 min, [M+H]⁺ 395.1, 397.1

The following compounds were prepared in a similar manner to ethyl 1-[(5-chloro-2-phenyl-1-benzofuran-7-yl)methyl]-5-methyl-1H-pyrazole-3-carboxylate from the appropriate intermediates:

| Description | Structure | Name | LC/MS data |
|---|---|---|---|
| 68 (D68) | 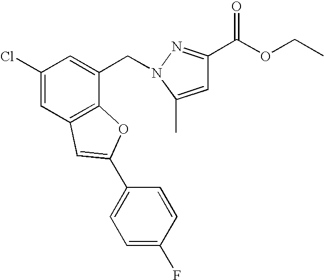 | Ethyl 1-{[5-chloro-2-(4- fluorophenyl)-1- benzofuran-7- yl]methyl}-5-methyl- 1H-pyrazole-3- carboxylate | Rt = 4.11 min, [M + H]⁺ 413.1, 415.1 |
| 69 (D69) | 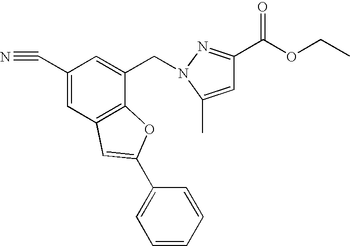 | Ethyl 1-[(5-cyano-2- phenyl-1-benzofuran- 7-yl)methyl]-5-methyl- 1H-pyrazole-3- carboxylate | Rt = 3.36 min, [M + H]⁺ 386.2, 387.2 |

### Description 70 (D70)

### Ethyl 1-{[5-chloro-2-(2,4-difluorophenyl)-1-benzofuran-7-yl]methyl}-5-methyl-1H-pyrazole-3-carboxylate

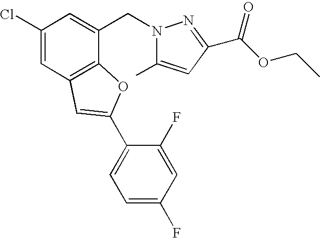

To solutions of 5-chloro-7-(chloromethyl)-2-(2,4-difluorophenyl)-1-benzofuran (assumed 5.75 mmol and 4.45 mmol) in dry N,N-dimethylformamide (20 ml and 15 ml) at room temperature under argon was added potassium carbonate (872 mg and 703 mg), sodium iodide (1.29 g and 933 mg) and ethyl 5-methyl-1H-pyrazole-3-carboxylate (Alfa Aesar, 974 mg and 703 mg) and the mixtures stirred overnight. The N,N-dimethylformamide was evaporated and the two reaction mixtures were combined using ethyl acetate (200 ml) and water (100 ml). Washed with water (3×100 ml) then dried (MgSO₄) and evaporated to a brown oil. The oil was dissolved in dichloromethane and applied to a Biotage Si 40+M column (pre-wetted with hexane) and eluted with hexane (250 ml) followed by 10% ethyl acetate/hexane (1 L) and 20% ethyl acetate/hexane (2 L) taking 20 ml fractions. Fractions 64-88 were evaporated and dried to afford the title compound as a pale yellow solid (1.47 g)

LC/MS Rt=3.77 min. Molecular ion observed [M+H]⁺ 431, consistent with molecular formula C₂₂H₁₇N₂O₃³⁵ ClF₂

### Example 1 (E1)

### 1-{[5-Chloro-2-(2,4-dichlorophenyl)-1-benzofuran-7-yl]methyl}-5-methyl-1H-pyrazole-3-carboxylic acid

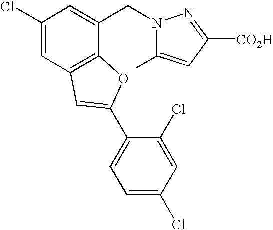

Solution of ethyl 1-{[5-chloro-2-(2,4-dichlorophenyl)-1-benzofuran-7-yl]methyl}-5-methyl-1H-pyrazole-3-carboxylate (0.738 g, 1.59 mmol) in EtOH (3.2 ml) and NaOH (aq.) (2M, 2.4 ml, 4.77 mmol) was stirred at 80° C. for 3 hours. After this time, EtOH was removed under reduced pressure. Residue was acidified using 2 M HCl and organics were extracted into EtOAc. Combined organics were dried over MgSO₄, filtered and concentrated under reduced pressure to give a yellow coloured solid.

LC/MS Rt=3.55 min [M+H]⁺ 435

The following compounds were prepared in a similar manner using the appropriate esters. In some cases the title compound was purified using MDAP.

| Example No. | Structure | Name | LC/MS Data |
|---|---|---|---|
| 2 (E2) | 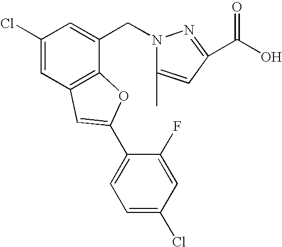 | 1-{[5-chloro-2-(4-chloro-2- fluorophenyl)-1- benzofuran-7-yl]methyl}-5- methyl-1H-pyrazole-3- carboxylic acid | Rt = 3.55 min [M + H]⁺ 419, 422 |
| 3 (E3) | 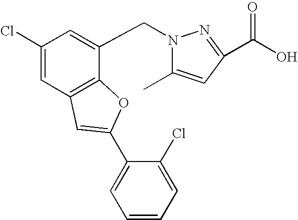 | 1-{[5-chloro-2-(2- chlorophenyl)-1- benzofuran-7-yl]methyl}-5- methyl-1H-pyrazole-3- carboxylic acid | Rt = 3.40 min |
| 4 (E4) | 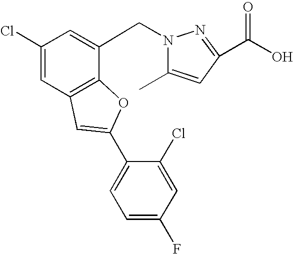 | 1-{[5-chloro-2-(2-chloro-4- fluorophenyl)-1- benzofuran-7-yl]methyl}-5- methyl-1H-pyrazole-3- carboxylic acid | Rt = 3.43 min, [M + H]⁺ 419, 421 |
| 5 (E5) | 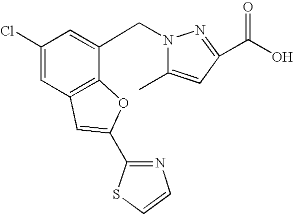 | 1-{[5-chloro-2-(1,3-thiazol- 2-yl)-1-benzofuran-7- yl]methyl}-5-methyl-1H- pyrazole-3-carboxylic acid | Rt = 2.69 min [M + H]⁺ 374, 376 |

### Example 6 (E6)

### 1-[(5-Chloro-2-phenyl-1-benzofuran-7-yl)methyl]-5-methyl-1H-pyrazole-3-carboxylic acid

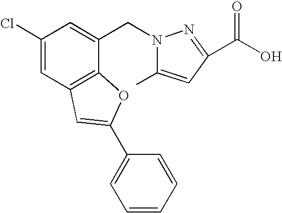

Solution of ethyl 1-[(5-chloro-2-phenyl-1-benzofuran-7-yl)methyl]-5-methyl-1H-pyrazole-3-carboxylate (4.08 g, 10.35 mmol) in EtOH (20 ml) was stirred at room temperature. NaOH (aq. soln.) (15 ml, 31.05 mmol, 2M) was added and the mixture heated to 60° C. for 2 hours. The solution was then allowed to cool to room temperature and water was added, a solid crashed out which was filtered off. The solid was treated with 2 M HCl and filtered again, washing with water. The remaining solution was acidified using 2M HCl and the organics were extracted into EtOAc. The combined organics were dried over MgSO₄, filtered and concentrated under reduced pressure to give a pale yellow coloured oily solid, giving a total of 3.97 g of the title compound.

LC/MS Rt=3.19 min, [M+H]⁺ 367, 369.

### Example 7 (E7)

### 6-[(5-Chloro-2-phenyl-1-benzofuran-7-yl)methyl]-2-pyridinecarboxylic acid

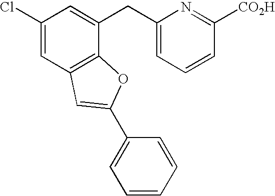

2M Sodium hydroxide (2 ml, 4 mmol) was added to a solution of ethyl 6-[(5-chloro-2-phenyl-1-benzofuran-7-yl)methyl]-2-pyridinecarboxylate (500 mg, 1.28 mmol) in ethanol (20 ml) and left at room temperature for one hour. The resulting solution was evaporated and the residue suspended in water/ether (40 ml of each) and acidified with 2M hydrochloric acid.

The organic phase was dried (magnesium sulphate), evaporated and triturated with 1:1 ether/hexane and filtered to give the title compound as a light coloured solid (416 mg).

LC/MS: Rt=3.37 min, [M+H]⁺ 364.1, 366.0

The following compounds were prepared in a similar manner to above by hydrolysis of the appropriate ester.

| Example | | | |
|---|---|---|---|
| No. | Structure | Name | LC/MS data |
| Example 8 (E8) | 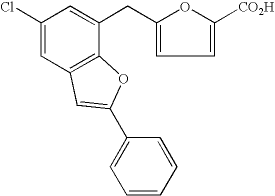 | 5-[(5-Chloro-2- phenyl-1- benzofuran-7- yl)methyl]-2- furancarboxylic acid | Rt = 3.43 min, [M + H]⁺ 353.1 |
| Example 9 (E9) | 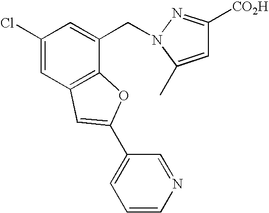 | 1-{[5-Chloro-2-(3- pyridinyl)-1- benzofuran-7- yl]methyl}-5-methyl- 1H-pyrazole-3- carboxylic acid | Rt = 2.44 min, [M + H]⁺ 368.1, 370.2 |
| Example 10 (E10) | 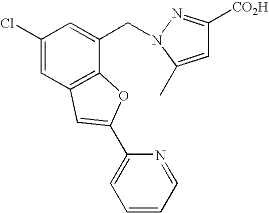 | 1-{[5-Chloro-2-(2- pyridinyl)-1- benzofuran-7- yl]methyl}-5-methyl- 1H-pyrazole-3- carboxylic acid | Rt = 2.67 min, [M + H]⁺ 368.1, 370.2 |
| Example 11 (E11) | 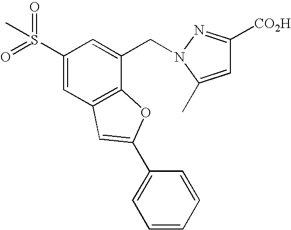 | 5-Methyl-1-{[5- (methylsulfonyl)-2- phenyl-1- benzofuran-7- yl]methyl}-1H- pyrazole-3- carboxylic acid | Rt = 2.63 min, [M + H]⁺ 411.2 |
| Example 12 (E12) | 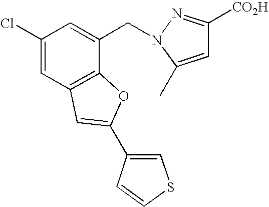 | 1-{[5-Chloro-2-(3- thienyl)-1- benzofuran-7- yl]methyl}-5-methyl- 1H-pyrazole-3- carboxylic acid | Rt = 3.08 min, [M + H]⁺ 373.1, 375.1 |
| Example 13 (E13) | 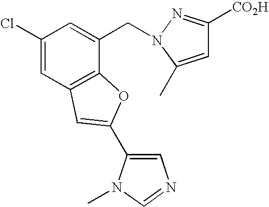 | 1-{[5-Chloro-2-(1- methyl-1H-imidazol- 5-yl)-1-benzofuran- 7-yl]methyl}-5- methyl-1H-pyrazole- 3-carboxylic acid | Rt = 1.89 min, [M + H]⁺ 371.2, 373.2 |
| Example 14 (E14) | 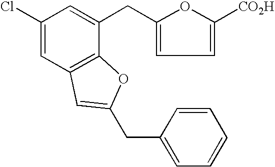 | 1-{[5-Chloro-2- (phenylmethyl)-1- benzofuran-7- yl]methyl}-5-methyl- 1H-pyrazole-3- carboxylic acid | Rt = 3.40 min, [M + H]⁺ 367.1 |
| Example 15 (E15) | 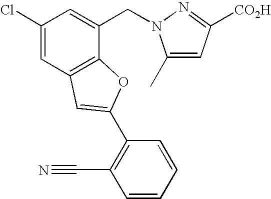 | 1-{[5-Chloro-2-(2- cyanophenyl)-1- benzofuran-7- yl]methyl}-5-methyl- 1H-pyrazole-3- carboxylic acid | Rt = 3.01 min, [M + H]⁺ 392.1 |
| Example 16 (E16) | 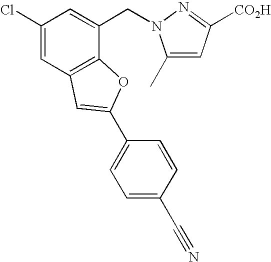 | 1-{[5-Chloro-2-(4- cyanophenyl)-1- benzofuran-7- yl]methyl}-5-methyl- 1H-pyrazole-3- carboxylic acid | Rt = 3.05 min, [M + H]⁺ 392.1 |

### Example 6b (E6b)

### 1-[(5-Chloro-2-phenyl-1-benzofuran-7-yl)methyl]-5-methyl-1H-pyrazole-3-carboxylic acid

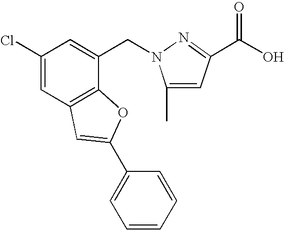

Ethyl 1-[(5-chloro-2-phenyl-1-benzofuran-7-yl)methyl]-5-methyl-1H-pyrazole-3-carboxylate (10.7 g, 27.3 mmol) was dissolved in hot ethanol (100 ml), NaOH 2M (27 ml) was added and the solution was heated at 50° C. for 2 hours. The solution was then cooled and the solvent evaporated. The solid obtained was treated with water, acidified with 2M HCl and extracted with warm ethyl acetate (3×300 ml). The combined extracts were dried (MgSO₄) and evaporated; the residue was azeotroped twice with toluene to give the title compound as white solid (9.94 g)

LC/MS Rt=3.21 min, [M+H]⁺ 367.1, 369.1.

The following compounds were prepared in a similar manner to 1-[(5-Chloro-2-phenyl-1-benzofuran-7-yl)methyl]-5-methyl-1H-pyrazole-3-carboxylic acid from the appropriate intermediates:

| Example | Structure | Name | LC/MS data |
|---|---|---|---|
| 17 (E17) | 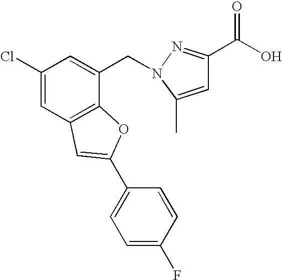 | 1-{[5-chloro-2-(4- fluorophenyl)-1- benzofuran-7-yl]methyl}-5- methyl-1H-pyrazole-3- carboxylic acid | Rt = 3.24 min, [M + H]⁺ 385, 387 |

### Example 18

### 1-[(5-Cyano-2-phenyl-1-benzofuran-7-yl)methyl]-5-methyl-1H-pyrazole-3-carboxylic acid

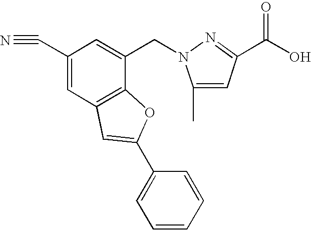

Ethyl 1-[(5-cyano-2-phenyl-1-benzofuran-7-yl)methyl]-5-methyl-1H-pyrazole-3-carboxylate (760 mg, 1.97 mmol) was dissolved in ethanol (25 ml), NaOH 2M (0.987 ml, 1.97 mmol) was added and the solution was stirred at room temperature for 4 hours during which time solid separated. THF (20 ml) was added to help dissolution; the solution was stirred at room temperature for 15 hours. The solvent was evaporated and the residue was treated with water, extracted with ethyl acetate (2×80 ml) to remove the starting material still present, acidified with 2M HCl and extracted with ethyl acetate (3×100 ml). The combined extracts were dried (MgSO₄) and evaporated to give an impure white solid. The solid was purified on the MDAP to give the title compound.

LC/MS Rt=2.9 min, [M+H]⁺ 358.1, 359.2, [M−H]⁻ 356.1, 357.2

### Example 19 (E19)

### 1-{[5-Chloro-2-(2,4-difluorophenyl)-1-benzofuran-7-yl]methyl}-5-methyl-1H-pyrazole-3-carboxylic acid

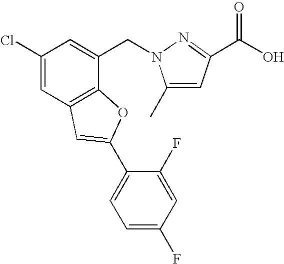

To a solution of ethyl 1-{[5-chloro-2-(2,4-difluorophenyl)-1-benzofuran-7-yl]methyl}-5-methyl-1H-pyrazole-3-carboxylate (1.47 g) in ethanol (27 ml) was added 2N sodium hydroxide (6.8 ml) and the mixture stirred at room temperature for one hour, then at 90° C. for fifteen minutes. The solution was evaporated and water was added to the residue. The aqueous suspension was acidified to pH1 with concentrated hydrochloric acid then filtered and washed with water and diethyl ether. The solid was dried at 60° C. under vacuum to afford the title compound as a white solid (887 mg). The washings were returned to a separating funnel and the aqueous removed. The organic layer was washed with water then dried (MgSO₄) and evaporated to a yellow solid. The solid was stirred in a small volume of diethyl ether for one hour then filtered off and washed with diethyl ether. The solid was dried at 60° C. under vacuum to afford the title compound as a white solid (199 mg).

LC/MS Rt=3.23 min. Molecular ion observed [M+H]⁺ 403, consistent with molecular formula C₂₀H₁₃N₂O₃³⁵ ClF₂

### Example 20 (E20)

### 1-{[5-chloro-2-(5-fluoro-2-pyridinyl)-1-benzofuran-7-yl]methyl}-5-methyl-1H-pyrazole-3-carboxylic acid

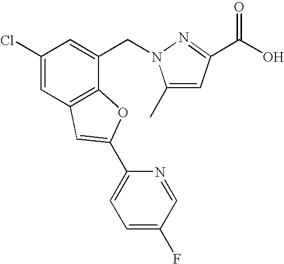

To a solution of ethyl 1-{[5-chloro-2-(5-fluoro-2-pyridinyl)-1-benzofuran-7-yl]methyl}-5-methyl-1H-pyrazole-3-carboxylate (285 mg) in ethanol (6 ml) was added 2N sodium hydroxide (1.38 ml) and the mixture stirred at 90° C. for thirty minutes. The mixture was evaporated and water was added to the residue. The aqueous suspension was acidified to pH1 with concentrated hydrochloric acid then filtered and washed with water. The solid was dried at 60° C. under vacuum to afford the title compound as a pale brown solid (259 mg).

LC/MS Rt=2.89 min. Molecular ion observed [M+H]⁺ 386, consistent with molecular formula C₁₉H₁₃N₃O₃³⁵ClF

### Example 21 (E21)

### 1-{[5-Chloro-2-(4-chlorophenyl)-1-benzofuran-7-yl]methyl}-5-methyl-1H-pyrazole-3-carboxylic acid

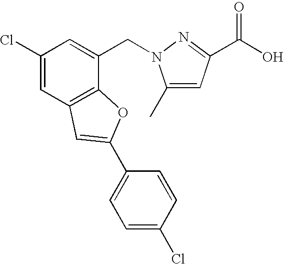

To a suspension of ethyl 1-{[5-chloro-2-(4-chlorophenyl)-1-benzofuran-7-yl]methyl}-5-methyl-1H-pyrazole-3-carboxylate (890 mg) in ethanol (18 ml) was added 2N sodium hydroxide (4.16 ml) and the mixture stirred at 90° C. for thirty minutes. The mixture was evaporated and water was added to the residue. The aqueous suspension was acidified to pH1 with concentrated hydrochloric acid then filtered and washed with water. The solid was dried at 60° C. under vacuum over sodium hydroxide. The solid was stirred in refluxing dichloromethane (25 ml) for thirty minutes and then allowed to cool. The solid was filtered off and washed with dichloromethane and dried under vacuum to afford the title compound (626 mg).

LC/MS Rt=3.37 min. Molecular ion observed [M+H]⁺ 401, consistent with molecular formula C₂₀H₁₄N₂O₃³⁵Cl₂

### Description 71 (D71)

### 5-Methyl-1-{[5-(methylsulfonyl)-2-phenyl-1-benzofuran-7-yl]methyl}-1H-pyrazole-3-carbonyl chloride

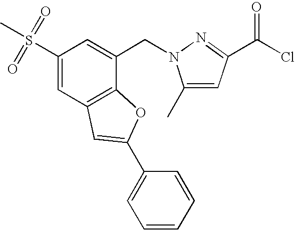

To a suspension of 5-methyl-1-{[5-(methylsulfonyl)-2-phenyl-1-benzofuran-7-yl]methyl}-1H-pyrazole-3-carboxylic acid (2.75 g) in dry dichloromethane (60 ml) and thionyl chloride (4.93 ml) was heated at 50° C. and stirred under argon for one hour. The solution was evaporated and co-evaporated from toluene.

### Description 72 (D72)

### 1-{[5-chloro-2-(2,4-dichlorophenyl)-1-benzofuran-7-yl]methyl}-5-methyl-1H-pyrazole-3-carbonyl chloride

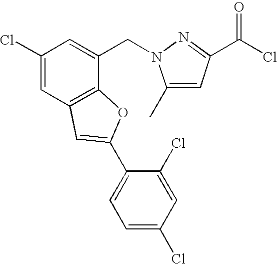

A solution of 1-{[5-chloro-2-(2,4-dichlorophenyl)-1-benzofuran-7-yl]methyl}-5-methyl-1H-pyrazole-3-carboxylic acid (0.400 g, 0.92 mmol) in dry DCM (3.68 ml) was stirred at room temperature under argon. SOCl₂ (0.335 ml, 4.59 mmol) was added and the solution stirred for 2½ hours at 60° C. After this time, the solvent was removed under reduced pressure and the residue was used directly in the next step.

The following compounds were prepared in a similar manner using the appropriate acid

| Description | Structure | Name |
|---|---|---|
| 73 (D73) | 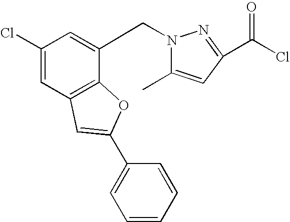 | 1-[(5-Chloro-2-phenyl-1-benzofuran-7- yl)methyl]-5-methyl-1H-pyrazole-3- carbonyl chloride |
| 74 (D74) | 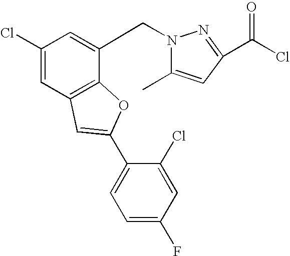 | 1-{[5-Chloro-2-(2-chloro-4-fluorophenyl)-1- benzofuran-7-yl]methyl}-5-methyl-1H- pyrazole-3-carbonyl chloride |

### Description 75 (D75)

### 2-(Trimethylsilyl)ethyl {1-[(5-chloro-2-phenyl-1-benzofuran-7-yl)methyl]-5-methyl-1H-pyrazol-3-yl}carbamate

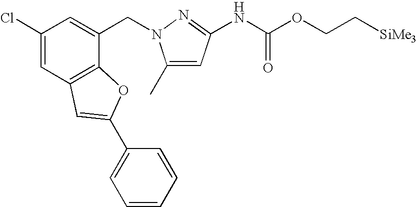

Solution of 1-[(5-chloro-2-phenyl-1-benzofuran-7-yl)methyl]-5-methyl-1H-pyrazole-3-carboxylic acid (3.79 g, 10.36 mmol) in dry PhCH₃ (40.0 ml) was stirred at room temperature under an atmosphere of argon. Diphenylphosphorylazide (2.453 ml, 11.39 mmol), Et₃N (1.729 ml, 12.43 mmol) and trimethylsilylmethanol (1.779 ml, 12.43 mmol) were added. The solution was heated to 80° C. under an atmosphere of argon for 24 hours. After this time, mixture was allowed to cool to room temperature and the mixture was left to stand for a further 24 hours. The mixture was then diluted with EtOAc (400 ml) and washed with NaHCO₃ (sat. aq. soln., 20 ml), brine was added to aid separation. Organics were separated and the aqueous layer washed with further EtOAc. The combined organics were dried over MgSO₄, filtered and concentrated under reduced pressure to give a brown solid. The residue was chromatographed [SiO₂, Hexane/EtOAc, 0-30%) to give the title compound (1.10 g)

LC/MS Rt=4.18 min [M+H]⁺ 482, 484.

### Description 76 (D76)

### 2-(Trimethylsilyl)ethyl (1-{[5-chloro-2-(4-cyanophenyl)-1-benzofuran-7-yl]methyl}-5-methyl-1H-pyrazol-3-yl)carbamate

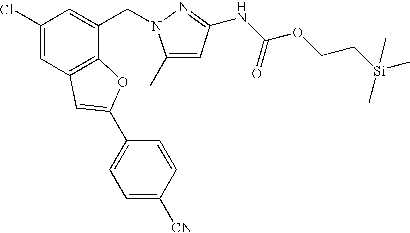

To a suspension of 1-{[5-chloro-2-(4-cyanophenyl)-1-benzofuran-7-yl]methyl}-5-methyl-1H-pyrazole-3-carboxylic acid (1.59 g) in dry toluene (17 ml) was added diphenylphosphoryl azide (0.962 ml) and the mixture stirred under argon at room temperature for thirty minutes. 2-Triethylsilyl ethanol (0.582 ml) and triethylamine (0.678 ml) were added and stirring continued for one hour. The reaction mixture was then heated at 90° C. overnight. The reaction mixture was diluted with ethyl acetate (100 ml) and washed with saturated sodium bicarbonate (50 ml) and 2:1 water:brine (150 ml) then dried (MgSO₄) and evaporated to afford a brown oil. The oil was dissolved in dichloromethane and applied to a Biotage Si 40+M column and purified using the Biotage SP4 (gradient method) to afford the title compound as a yellow: orange foam (613 mg).

LC/MS Rt=4.04 min. Molecular ion observed [M+H]⁺ 507, consistent with molecular formula C₂₆H₂₇N₄O₃³⁵ClSi

### Description 77 (D77)

### 2-(Trimethylsilyl)ethyl {1-[(5-cyano-2-phenyl-1-benzofuran-7-yl)methyl]-5-methyl-1H-pyrazol-3-yl}carbamate

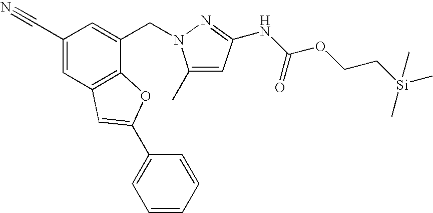

To 1-[(5-cyano-2-phenyl-1-benzofuran-7-yl)methyl]-5-methyl-1H-pyrazole-3-carboxylic acid (400 mg) in dry toluene (4.5 ml) was added diphenylphosphoryl azide (0.266 ml) and the mixture stirred under argon at room temperature for thirty minutes. 2-Triethylsilyl ethanol (0.241 ml) and triethylamine (0.187 ml) were added and stirring continued for thirty minutes. The reaction mixture was then heated at 90° C. overnight. The reaction mixture was diluted with ethyl acetate and washed with saturated sodium bicarbonate then dried (MgSO₄) and evaporated to afford a brown oily foam. Applied to a Biotage Si 25+M column and purified using the Biotage SP4 (gradient method) to afford the title compound (190 mg).

LC/MS Rt=3.90 min. Molecular ion observed [M+H]⁺ 473, consistent with molecular formula C₂₆H₂₈N₄O₃Si

### Description 78 (D78)

### 1-[(5-Chloro-2-phenyl-1-benzofuran-7-yl)methyl]-5-methyl-1H-pyrazol-3-amine

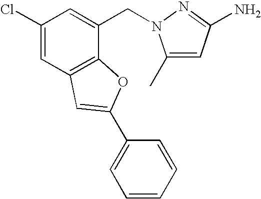

Solution of 2-(trimethylsilyl)ethyl {1-[(5-chloro-2-phenyl-1-benzofuran-7-yl)methyl]-5-methyl-1H-pyrazol-3-yl}carbamate (0.900 g, 1.87 mmol) in dry THF (7.5 ml) was stirred at room temperature under an atmosphere of argon. TBAF (1M in THF, 2.06 ml, 2.06 mmol) was added and the solution stirred for a further ½ hour. After ˜2½ hours more TBAF (0.5 ml) was added. The solution was stirred for 20 hours at room temperature. Mixture was diluted with EtOAc and washed with water (×3). Organics were dried over MgSO₄, filtered and concentrated under reduced pressure to give a pale yellow coloured oil. Oil was washed with Et₂O to give a yellow coloured solid. (0.525 g)

LC/MS Rt=3.06 min [M+H]⁺ 338, 340.

### Description 79 (D79)

### 4-{7-[(3-Amino-5-methyl-1H-pyrazol-1-yl)methyl]-5-chloro-1-benzofuran-2-yl}benzonitrile

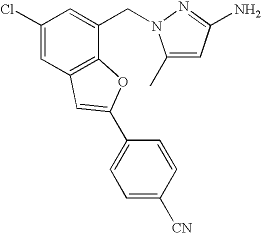

To a solution of 2-(trimethylsilyl)ethyl (1-{[5-chloro-2-(4-cyanophenyl)-1-benzofuran-7-yl]methyl}-5-methyl-1H-pyrazol-3-yl)carbamate (613 mg) in dry tetrahydrofuran (3 ml) under argon was added tetrabutylammonium fluoride solution (1M in tetrahydrofuran, 2.42 ml) and the reaction mixture heated at 50° C. The mixture was cooled and the tetrahydrofuran evaporated. The residue was diluted with ethyl acetate (50 ml) and washed with water whereby a yellow precipitate was formed. The solid was filtered off and dried at 50° C. under vacuum to afford the title compound (371 mg).

LC/MS Rt=2.85 min. Molecular ion observed [M+H]⁺ 363, consistent with molecular formula C₂₀H₁₅N₄O³⁵Cl

### Description 80 (D80)

### 7-[(3-Amino-5-methyl-1H-pyrazol-1-yl)methyl]-2-phenyl-1-benzofuran-5-carbonitrile

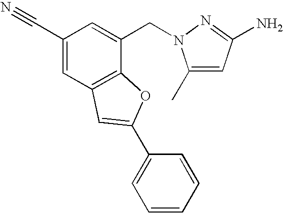

To a solution of 2-(trimethylsilyl)ethyl {1-[(5-cyano-2-phenyl-1-benzofuran-7-yl)methyl]-5-methyl-1H-pyrazol-3-yl}carbamate (142 mg) in dry tetrahydrofuran (1 ml) under argon was added tetrabutylammonium fluoride solution (1M in tetrahydrofuran, 0.6 ml) and the reaction mixture heated at 50° C. for one hour. The mixture was cooled and the tetrahydrofuran evaporated. The residue was dissolved in ethyl acetate (20 ml) and washed with water (3×15 ml) then dried (MgSO₄) and evaporated to afford the title compound as a white solid (92 mg).

LC/MS Rt=2.69 min. Molecular ion observed [M+H]⁺ 329, consistent with molecular formula C₂₀H₁₆N₄O

### Description 81 (D81)

### 1-[(5-Chloro-2-phenyl-1-benzofuran-7-yl)methyl]-5-methyl-1H-pyrazol-3-amine

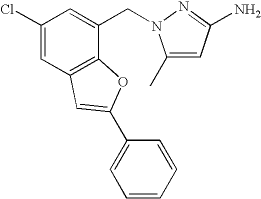

A suspension of 1-[(5-chloro-2-phenyl-1-benzofuran-7-yl)methyl]-5-methyl-1H-pyrazole-3-carboxylic acid (918 mg) in dry dichloromethane (35 ml) and thionyl chloride (1.83 ml) was heated at 50° C. whereby a solution was obtained and stirred under argon for ninety minutes. The solution was evaporated and co-evaporated from toluene to afford a yellow solid. The solid was suspended in acetone (15 ml) and treated with a solution of sodium azide (815 mg) in water (2 ml). The mixture was partitioned between water and toluene and extracted twice with dichloromethane. The combined organic layer was washed with water then dried (MgSO₄) and evaporated to remove most of the dichloromethane. The solution was heated to 100° C. for one hour then treated with concentrated hydrochloric acid (2 ml) and stirred at 110° C. for three hours. The reaction mixture was evaporated, the solid residue triturated with dichloromethane, filtered off, washed with dichloromethane and dried at 50° C. under vacuum to afford the title compound (622 mg).

LC/MS Rt=3.01 min. Molecular ion observed [M+H]⁺ 338, consistent with molecular formula C₁₉H₁₆N₃O³⁵Cl

### Description 82 (D82)

### 1,1-Dimethylethyl 4-({[(1-{[5-chloro-2-(2,4-dichlorophenyl)-1-benzofuran-7-yl]methyl}-5-methyl-1H-pyrazol-3-yl)carbonyl]amino}methyl)-1-piperidinecarboxylate

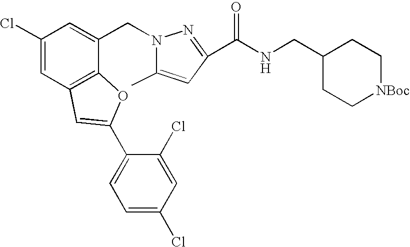

Solution of 1-{[5-chloro-2-(2,4-dichlorophenyl)-1-benzofuran-7-yl]methyl}-5-methyl-1H-pyrazole-3-carbonyl chloride (0.145 g, 0.31 mmol) in dry DCM (2.0 ml) was stirred at room temperature under an atmosphere of argon. Et₃N (0.086 ml, 0.62 mmol) was added to the solution. 4-(Aminomethyl)-1-boc-piperidine (0.098 g, 0.46 mmol) was added to the mixture and stirring continued at room temperature overnight. After this time, solvent was removed under reduced pressure and the residue purified using MDAP to give the title compound.

LC/MS Rt=4.36 min [M+H]⁺ 633.

### Description 83 (D83)

### 1,1-Dimethylethyl 4-({[(1-{[5-chloro-2-(2-chlorophenyl)-1-benzofuran-7-yl]methyl}-5-methyl-1H-pyrazol-3-yl)carbonyl]amino}methyl)-1-piperidinecarboxylate

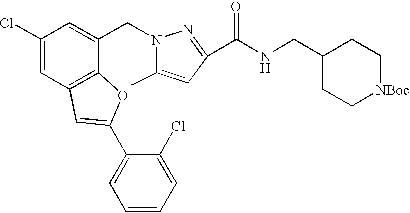

Solution of 1-{[5-chloro-2-(2-chlorophenyl)-1-benzofuran-7-yl]methyl}-5-methyl-1H-pyrazole-3-carboxylic acid (0.150 g, 0.38 mmol) in dry DCM (1.3 ml) was stirred at room temperature under an atmosphere of argon. 4-(aminomethyl)-1-boc-piperidine (0.098 g, 0.46 mmol), EDAC (0.087 g, 0.46 mmol) and HOBt (0.062 g, 0.46 mmol) were added, and the solution stirred at room temperature for 19 hours. After this time, the solution was diluted with DCM and washed with water. Organics were dried over MgSO₄, filtered and concentrated under reduced pressure. The residue was chromatographed [SiO₂ Hexane/EtOAc 25-75%] to give the title compound.

LC/MS Rt=4.06 min, [M+H]⁺ 597.

### Description 84 (D84)

### 1,1-Dimethylethyl 4-{[({5-[(5-chloro-2-phenyl-1-benzofuran-7-yl)methyl]-2-furanyl}carbonyl)amino]methyl}-1-piperidinecarboxylate

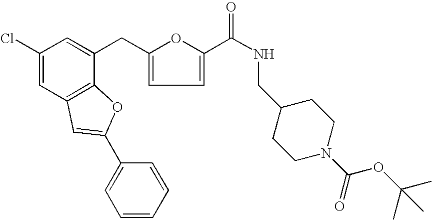

Oxalyl chloride (0.2 ml) was added to a suspension of 1-{[5-chloro-2-(2-pyridinyl)-1-benzofuran-7-yl]methyl}-5-methyl-1H-pyrazole-3-carboxylic acid (100 mg, 0.28 mmol) and DMF (1 drop) in dichloromethane (5 ml) and left at room temperature for 1 hour. The solution was evaporated and azeotroped with toluene (10 ml). The residue was dissolved in dichloromethane (3 ml) and a solution of N-Boc-4-aminomethyl piperidine (70 mg, 0.33 mmol) in pyridine (0.2 ml) was added and stirred for 30 minutes. The resulting solution was diluted with EtOAc (40 ml) washed with water (20 ml), dried (magnesium sulphate), evaporated and purified by flash chromatography on a Biotage column eluting with 40% ethyl acetate in hexane to give the title compound as a colourless gum (115 mg).

LC/MS Rt=3.94 min, [M+H]⁺ 452.1, 549.2

The following compounds were prepared in a similar manner by reaction of the appropriate acid with oxalyl chloride then treatment with an amine and pyridine.

| | | | LC/MS |
|---|---|---|---|
| Description | Structure | Name | data |
| 85 (D85) | 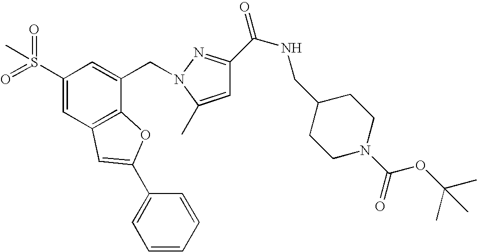 | 1,1-Dimethylethyl 4- ({[(5-methyl-1-{[5- (methylsulfonyl)-2- phenyl-1-benzofuran- 7-yl]methyl}-1H- pyrazol-3- yl)carbonyl]amino}methyl)-1- piperidinecarboxylate | Rt = 3.27 min, [M + H]⁺ 607.3 |
| 86 (D86) | 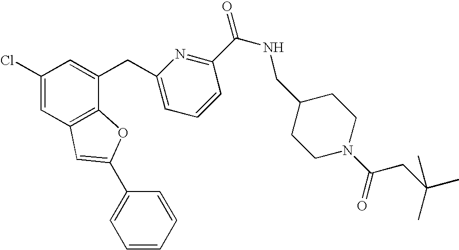 | 1,1-Dimethylethyl 4- {[({6-[(5-chloro-2- phenyl-1-benzofuran- 7-yl)methyl]-2- pyridinyl}carbonyl)amino] methyl}-1- piperidinecarboxylate | Rt = 4.15 min [M + H]⁺ 560.3 |
| 87 (D87) | 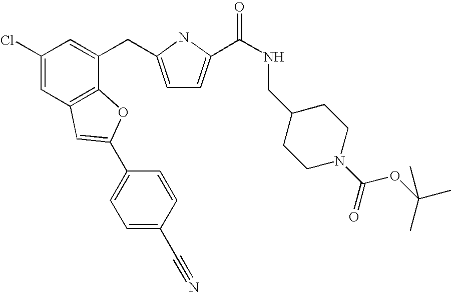 | 1,1-Dimethylethyl 4- ({[(5-{[5-chloro-2-(4- cyanophenyl)-1- benzofuran-7- yl]methyl}-2- furanyl)carbonyl] amino}methyl)-1- piperidinecarboxylate | Rt = 3.80 min [M + H- BOC]⁺ 474.1 |
| 88 (D88) | 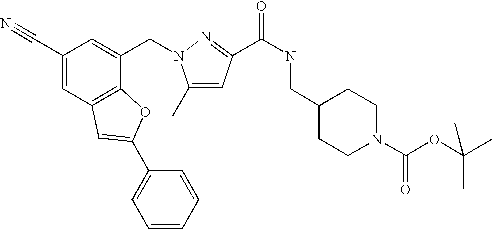 | 1,1-Dimethylethyl 4- {[({1-[(5-cyano-2- phenyl-1- benzofuran-7- yl)methyl]-5-methyl- 1H-pyrazol-3- yl}carbonyl)amino] methyl}-1- piperidinecarboxylate | Rt = 3.50 min [M + H]⁺ 554.3 |

### Description 89 (D89)

### 1-{[5-Chloro-2-(2,4-difluorophenyl)-1-benzofuran-7-yl]methyl}-N-[4-(hydroxymethyl)phenyl]-5-methyl-1H-pyrazole-3-carboxamide

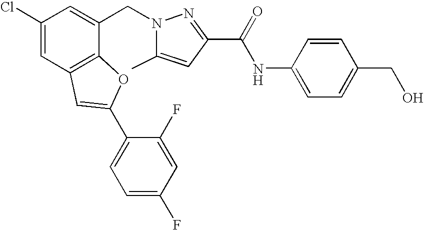

To a semi-solution of 1-{[5-chloro-2-(2,4-difluorophenyl)-1-benzofuran-7-yl]methyl}-5-methyl-1H-pyrazole-3-carboxylic acid (200 mg) in N,N-dimethylformamide (4 ml) was added N-ethylmorpholine (0.253 ml), 4-iminobenzyl alcohol (73 mg), 1-hydroxybenzotriazole hydrate (104 mg) and N-(3-dimethylaminopropyl)-N′-ethylcarbodiimide (114 mg). After thirty minutes the reaction mixture was in solution and was stirred overnight. The reaction mixture was diluted with ethyl acetate (40 ml) and washed with saturated sodium bicarbonate (40 ml) and water (40 ml) then dried (MgSO₄) and evaporated to afford the title compound as an off-white solid.

LC/MS Rt=3.57 min. Molecular ion observed [M+H]⁺ 508, consistent with molecular formula C₂₇H₂₀N₃O₃³⁵ ClF₂

### Description 90 (D90)

### 1-[(5-Chloro-2-phenyl-1-benzofuran-7-yl)methyl]-N-[4-(hydroxymethyl)phenyl]-5-methyl-1H-pyrazole-3-carboxamide

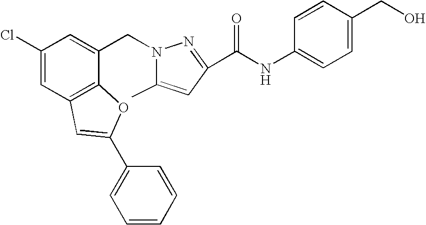

1-[(5-chloro-2-phenyl-1-benzofuran-7-yl)methyl]-5-methyl-1H-pyrazole-3-carboxylic acid (1.50 g, 4.09 mmol) was dissolved in dry DMF (14 ml). EDAC (942 mg, 4.91 mmol), HOBt (664 mg, 4.91 mmol) and 4-amino-benzylalcohol (607 mg, 4.91 mmol) were added. The reaction solution was stirred under an atmosphere of argon at room temperature for 17 hours (overnight). After this time, a further 0.5 equiv. of aniline was added and the reaction stirred at room temperature under argon for a further 2 hours. The reaction was diluted with EtOAc and washed with sat. soln. NaHCO₃(10 ml). A precipitate formed in the aqueous layer. The organics were washed with further water (3×20 ml). Organics were dried over MgSO₄, filtered and concentrated to give a yellow solid. The residue was washed with the minimum amount of DCM. DCM took on a yellow colour, leaving a pale yellow solid. Process was repeated to give the title compound. (1.06 g)

LC/MS Rt=3.34 min, [M+H]⁺ 472, 474

### Description 91 (D91)

### 6-[(5-Chloro-2-phenyl-1-benzofuran-7-yl)methyl]-N-[4-(hydroxymethyl)phenyl]-2-pyridinecarboxamide

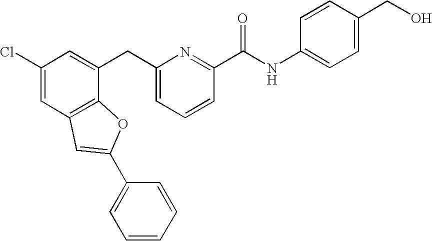

6-[(5-chloro-2-phenyl-1-benzofuran-7-yl)methyl]-2-pyridinecarboxylic acid (400 mg, 1.1 mmol), 4-aminobenzylalcohol (270 mg, 2.2 mmol), EDAC (281 mg, 1.5 mmol) and HOBt (184 mg, 1.2 mmol) were stirred in DMF (5 ml) at room temperature for 4 hours. The mixture was then diluted with water/ether (50 ml of each). The organic layer was washed with 60 ml sat. NaHCO₃, 30 ml HCl (aq) 2M and water (2×20 ml). the organics were then dried over MgSO₄ and evaporated to give a yellow solid. (476 mg).

LC/MS Rt=3.79 min

### Description 92 (D92)

### 1-[(5-chloro-2-phenyl-1-benzofuran-7-yl)methyl]-N-(5-ethenyl-2-pyridinyl)-5-methyl-1H-pyrazole-3-carboxamide

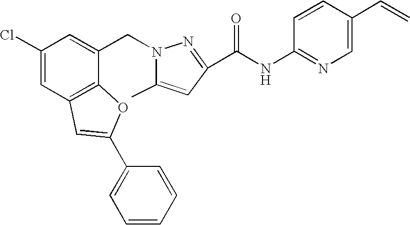

1-[(5-chloro-2-phenyl-1-benzofuran-7-yl)methyl]-5-methyl-1H-pyrazole-3-carbonyl chloride (630 mg, 1.637 mmol) was dissolved in dry DCM (3.3 ml). 5-ethenyl-2-pyridinamine (98 mg, 0.819 mmol) and Et₃N (0.5 ml) were added. The solution was stirred under an atmosphere of argon, at room temperature for 2 hours. The reaction mixture was concentrated under reduced pressure. Ethanol (10 ml) and NaOH (2M, 4.5 ml) were added to the solution. The reaction mixture was stirred at room temperature for 1 hour. Further NaOH (2M, 4.5 ml) was added and the reaction mixture was stirred at room temperature under argon for a further 17 hours (overnight). The solution was analyzed by LC/Ms and then left to stir for a total of 75 hours. The reaction mixture was then concentrated under reduced pressure. The solution was diluted with DCM and NaHCO₃ (aq. soln.). Organic layer was washed with water. The organics were dried over MgSO₄, filtered and concentrated to give a solid. The compound was chromatographed [SiO₂, 0-20% EtOAc in Hexane] to give the title compound. (193 mg)

LC/MS Rt=3.95 min, [M+H]⁺ 469, 471

### Description 93 (D93)

### 1-[(5-Chloro-2-phenyl-1-benzofuran-7-yl)methyl]-N-(5-formyl-2-pyridinyl)-5-methyl-1H-pyrazole-3-carboxamide

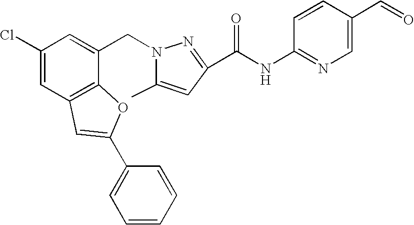

1-[(5-chloro-2-phenyl-1-benzofuran-7-yl)methyl]-N-(5-ethenyl-2-pyridinyl)-5-methyl-1H-pyrazole-3-carboxamide (193 mg, 0.412 mmol) was dissolved in THF (1.2 ml) and H₂O (1.2 ml). 2 drops of OsO₄ and NalO₄ (220 mg, 1.03 mmol) were added to the mixture. The mixture was stirred at room temperature for a total of 16 hours. The reaction mixture was then diluted with DCM and water until the solid dissolved, and filtered through a hydrophobic frit, fitted with an Na₂SO₄ drying capsule. The aqueous layer was washed with further DCM (×2). The organics were concentrated under reduced pressure to give a white foam. The residue was chromatographed [SiO₂, 20-50% EtOAc in Hexane] to give the title compound. (56 mg)

LC/MS Rt=3.73 min [M+H]⁺ 471, 473

### Description 94 (D94)

### 1-{[5-Chloro-2-(2,4-difluorophenyl)-1-benzofuran-7-yl]methyl}-N-(4-formylphenyl)-5-methyl-1H-pyrazole-3-carboxamide

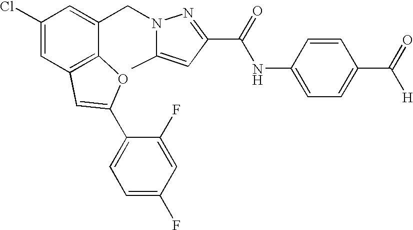

To a suspension of 1-{[5-chloro-2-(2,4-difluorophenyl)-1-benzofuran-7-yl]methyl}-N-[4-(hydroxymethyl)phenyl]-5-methyl-1H-pyrazole-3-carboxamide (0.5 mmol) in dichloromethane (10 ml) was added, portionwise, Dess-Martin periodinane (210 mg) to give a pale cloudy mixture. The reaction mixture was stirred for forty minutes then cooled to 0° C. and treated with saturated sodium bicarbonate (4 ml) and 10% sodium thiosulphate (6 ml). The mixture was extracted with dichloromethane and the organic layer was dried (MgSO₄) and evaporated to afford the title compound as a pale brown foam (269 mg).

LC/MS Rt=3.93 min. Molecular ion observed [M+H]⁺ 506, consistent with molecular formula C₂₇H₁₈N₃O₃³⁵ ClF₂

The following compound was prepared in a similar manner from the appropriate benzyl alcohol:

| | | | LC/MS |
|---|---|---|---|
| Description | Structure | Name | data |
| 95 (D95) | 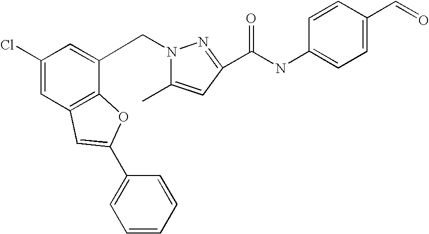 | 1-[(5-chloro-2-phenyl-1- benzofuran-7- yl)methyl]-N-(4- formylphenyl)-5-methyl- 1H-pyrazole-3- carboxamide | Rt = 3.67 min [MNa⁺] 492, 494 |
| 96 (D96) | 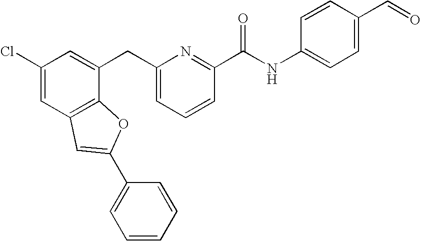 | 6-[(5-chloro-2-phenyl-1- benzofuran-7- yl)methyl]-N-(4- formylphenyl)-2- pyridinecarboxamide | LC/MS Rt = 4.16 min |

### Description 97 (D97)

### 1,1-Dimethylethyl 4-[(4-{[({[5-chloro-2-(2,4-difluorophenyl)-1-benzofuran-7-yl]methyl}-5-methyl-1H-pyrazol-3-yl)carbonyl]amino}phenyl)methyl]-1-piperazinecarboxylate

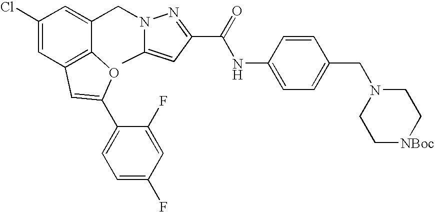

To a solution of 1-{[5-chloro-2-(2,4-difluorophenyl)-1-benzofuran-7-yl]methyl}-N-(4-formylphenyl)-5-methyl-1H-pyrazole-3-carboxamide (0.5 mmol) in dichloromethane was added BOC-piperazine (111 mg) and the solution stirred under argon for thirty minutes. Acetic acid (0.16 ml) and sodium triacetoxyborohydride (126 mg) were added and after stirring for seventy five minutes, BOC-piperazine (56 mg) was added, and after a further one hundred and five minutes, sodium triacetoxyborohydride (63 mg) was added and the mixture stirred overnight. The reaction mixture was diluted with dichloromethane (30 ml) and washed with 1:1 saturated sodium bicarbonate: water (25 ml), water (25 ml), and brine (25 ml) then dried (MgSO₄) and evaporated to afford a foam (323 mg). The foam was dissolved in dichloromethane and applied to a Biotage Si 25+S column (pre-wetted with hexane) and eluted with 50% ethyl acetate/hexane (500 ml) taking 10 ml fractions. Fractions 15-39 were evaporated and dried to afford the title compound as a white foam (214 mg).

LC/MS Rt=2.83 min. Molecular ion observed [M+H]⁺ 676, consistent with molecular formula C₃₆H₃₆N₅O₄³⁵ ClF₂

### Description 98 (D98)

### 1,1-Dimethylethyl 4-({[(1-{[5-chloro-2-(2,4-difluorophenyl)-1-benzofuran-7-yl]methyl}-5-methyl-1H-pyrazol-3-yl)carbonyl]amino}methyl)-1-piperidinecarboxylate

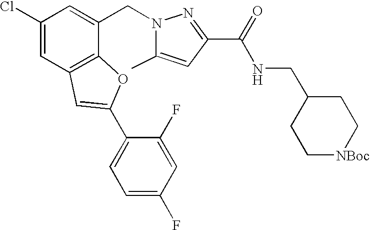

To a semi-solution of 1-{[5-chloro-2-(2,4-difluorophenyl)-1-benzofuran-7-yl]methyl}-5-methyl-1H-pyrazole-3-carboxylic acid (100 mg) in N,N-dimethylformamide (4 ml) was added N-ethylmorpholine (0.126 ml), 1,1-dimethylethyl 4-(aminomethyl)-1-piperidinecarboxylate (64 mg), 1-hydroxybenzotriazole hydrate (52 mg) and N-(3-dimethylaminopropyl)-N′-ethylcarbodiimide (57 mg). After thirty minutes the reaction mixture was in solution and was stirred overnight. The reaction mixture was diluted with ethyl acetate (40 ml) and washed with saturated sodium bicarbonate (25 ml) and water (2×25 ml) then dried (MgSO₄) and evaporated to afford a white foam. The foam was dissolved in dichloromethane and applied to a Biotage Si 25+S column (pre-wetted with hexane) and eluted with 40% ethyl acetate/hexane (500 ml) taking 5 ml fractions. Fractions 47-65 were evaporated and dried to afford the title compound as a white foam (110 mg).

LC/MS Rt=3.96 min. Molecular ion observed [M+H]⁺ 599, consistent with molecular formula C₃₁H₃₃N₄O₄³⁵ ClF₂

### Description 99 (D99)

### 1,1-Dimethylethyl 4-({[(1-{[5-chloro-2-(4-chlorophenyl)-1-benzofuran-7-yl]methyl}-5-methyl-1H-pyrazol-3-yl)carbonyl]amino}methyl)-1-piperidinecarboxylate

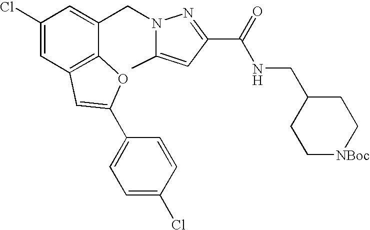

To a solution of 1-{[5-chloro-2-(4-chlorophenyl)-1-benzofuran-7-yl]methyl}-5-methyl-1H-pyrazole-3-carboxylic acid (50 mg) in N,N-dimethylformamide (2 ml) was added N-ethylmorpholine (0.064 ml), 1,1-dimethylethyl 4-(aminomethyl)-1-piperidinecarboxylate (32 mg), 1-hydroxybenzotriazole hydrate (26 mg) and N-(3-dimethylaminopropyl)-N′-ethylcarbodiimide (29 mg). After two hours the reaction mixture was in solution and was stirred overnight. The reaction mixture was diluted with ethyl acetate (30 ml) and washed with saturated sodium bicarbonate (20 ml) and water (3×15 ml) then dried (MgSO₄) and evaporated to afford a yellow oil. The oil was dissolved in dichloromethane and applied to a Biotage Si 12+M column and purified using the Biotage SP4 (gradient method) to afford the title compound as an off-white coloured foam (68 mg).

LC/MS Rt=4.04 min. Molecular ion observed [M+H⁺] 597, consistent with molecular formula C₃₁H₃₄N₄O₄³⁵Cl₂

### Description 100 (D100)

### 1,1-Dimethylethyl 4-({[(1-{[5-chloro-2-(4-cyanophenyl)-1-benzofuran-7-yl]methyl}-5-methyl-1H-pyrazol-3-yl)carbonyl]amino}methyl)-1-piperidinecarboxylate

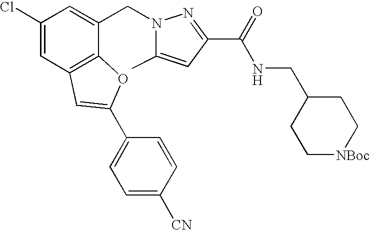

To a solution of 1-{[5-chloro-2-(4-cyanophenyl)-1-benzofuran-7-yl]methyl}-5-methyl-1H-pyrazole-3-carboxylic acid (150 mg) in N,N-dimethylformamide (6 ml) was added N-ethylmorpholine (0.193 ml), 1,1-dimethylethyl 4-(aminomethyl)-1-piperidinecarboxylate (98 mg), 1-hydroxybenzotriazole hydrate (80 mg) and N-(3-dimethylaminopropyl)-N′-ethylcarbodiimide (87 mg) and the reaction mixture stirred overnight. The reaction mixture was diluted with ethyl acetate (90 ml) and washed with saturated sodium bicarbonate (60 ml) and water (3×30 ml) then dried (MgSO₄) and evaporated to afford an orange oil. The oil was dissolved in dichloromethane and applied to a Biotage Si 12+M column and purified using the Biotage SP4 (gradient method) to afford the title compound as an orange oil.

LC/MS Rt=3.72 min. Molecular ion observed [M+H]⁺ 588, consistent with molecular formula C₃₂H₃₄N₅O₄³⁵Cl

### Description 101 (D101)

### Methyl 6-[({1-[(5-chloro-2-phenyl-1-benzofuran-7-yl)methyl]-5-methyl-1H-pyrazol-3-yl}carbonyl)amino]-3-pyridinecarboxylate

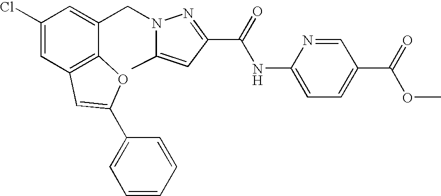

A suspension of 1-[(5-chloro-2-phenyl-1-benzofuran-7-yl)methyl]-5-methyl-1H-pyrazole-3-carboxylic acid (309 mg) in dry dichloromethane (12 ml) and thionyl chloride (0.615 ml) was heated at 50° C. under argon for one hour to give a solution. The solution was evaporated to dryness and the residue dissolved in dichloromethane (6 ml) and cooled to 0° C. Triethylamine (0.615 ml) and methyl 6-amino-3-pyridinecarboxylate (154 mg) were added and after one hour, the reaction mixture was diluted with dichloromethane and washed with saturated sodium bicarbonate (20 ml), water (20 ml), 2N hydrochloric acid (20 ml), water (20 ml), saturated sodium bicarbonate (20 ml) and brine (20 ml). The organic layer was then dried (MgSO₄) and evaporated to a white solid. The solid was dissolved in dichloromethane and applied to a Biotage Si 40+M column and purified using the Biotage SP4 (gradient method) and dried to afford the title compound as a white solid (162 mg).

LC/MS Rt=4.09 min. Molecular ion observed [M+H]⁺ 501, consistent with molecular formula C₂₇H₂₁N₄O₄³⁵Cl

### Description 102 (D102)

### 6-[({1-[(5-Chloro-2-phenyl-1-benzofuran-7-yl)methyl]-5-methyl-1H-pyrazol-3-yl}carbonyl)amino]-3-pyridinecarboxylic acid

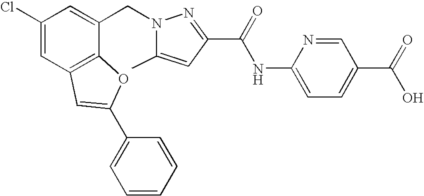

To a suspension of methyl 6-[({1-[(5-chloro-2-phenyl-1-benzofuran-7-yl)methyl]-5-methyl-1H-pyrazol-3-yl}carbonyl)amino]-3-pyridinecarboxylate (162 mg) in ethanol (5 ml) was added 2N sodium hydroxide (0.65 ml) and the mixture stirred at 90° C. for thirty minutes. The mixture was evaporated and water was added to the residue. The aqueous suspension was acidified to pH1 with concentrated hydrochloric acid then filtered and washed with water. The solid was dried at 50° C. under vacuum to afford the title compound (128 mg).

LC/MS Rt=3.05 min. Molecular ion observed [M+H]⁺ 487, consistent with molecular formula C₂₆H₁₉N₄O₄³⁵Cl

### Description 103 (D103)

### 1,1-Dimethylethyl 4-{[({6-[({1-[(5-chloro-2-phenyl-1-benzofuran-7-yl)methyl]-5-methyl-1H-pyrazol-3-yl}carbonyl)amino]-3-pyridinyl}carbonyl)amino]methyl}-1-piperidinecarboxylate

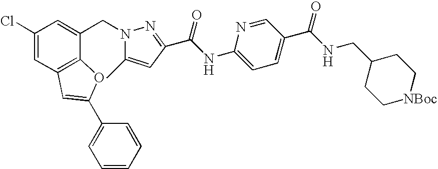

To a suspension of 6-[({1-[(5-chloro-2-phenyl-1-benzofuran-7-yl)methyl]-5-methyl-1H-pyrazol-3-yl}carbonyl)amino]-3-pyridinecarboxylic acid (assumed 0.324 mmol) in N,N-dimethylformamide (6 ml) was added N-ethylmorpholine (0.165 ml), 1,1-dimethylethyl 4-(aminomethyl)-1-piperidinecarboxylate (83 mg), 1-hydroxybenzotriazole hydrate (68 mg) and N-(3-dimethylaminopropyl)-N′-ethylcarbodiimide (75 mg) and the reaction mixture stirred overnight. The reaction mixture was diluted with ethyl acetate (60 ml) and washed with saturated sodium bicarbonate (40 ml) and water (2×30 ml) then dried (MgSO₄) and evaporated to afford a yellow oil. The oil was dissolved in dichloromethane and applied to a Biotage Si 25+S column and purified using the Biotage SP4 (gradient method) to afford the title compound (62 mg).

LC/MS Rt=4.03 min. Molecular ion observed [M+H]⁺ 683, consistent with molecular formula C₃₇H₃₉N₆O₅³⁵Cl

### Description 104 (D104)

### 1,1-Dimethylethyl (3R)-3-[({1-[(5-chloro-2-phenyl-1-benzofuran-7-yl)methyl]-5-methyl-1H-pyrazol-3-yl}carbonyl)amino]-1-pyrrolidinecarboxylate

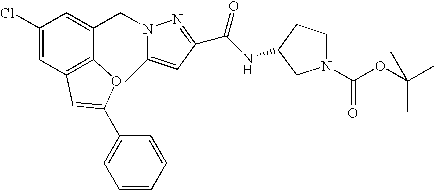

To a suspension of 1-[(5-chloro-2-phenyl-1-benzofuran-7-yl)methyl]-5-methyl-1H-pyrazole-3-carboxylic acid (200 mg) in N,N-dimethylformamide (8 ml) was added N-ethylmorpholine (0.280 ml), (R)-(+)-N-Boc-3-amino pyrrolidine (0.112 ml), 1-hydroxybenzotriazole hydrate (92 mg) and N-(3-dimethylaminopropyl)-N′-ethylcarbodiimide (127 mg) and the reaction mixture was stirred overnight. The reaction mixture was diluted with ethyl acetate (40 ml) and washed with saturated sodium bicarbonate (25 ml) and water (2×25 ml) then dried (MgSO₄) and evaporated to afford the title compound (129 mg).

LC/MS Rt=3.71 min. Molecular ion observed [M+H]⁺ 535, consistent with molecular formula C₂₉H₃₁N₄O₄³⁵Cl

### Description 105 (D105)

### 1,1-Dimethylethyl (3R)-3-[({1-[(5-chloro-2-phenyl-1-benzofuran-7-yl)methyl]-5-methyl-1H-pyrazol-3-yl}amino)carbonyl]-1-piperidinecarboxylate

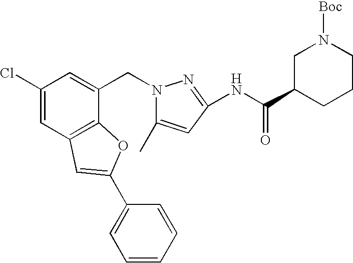

Solution of 1-[(5-chloro-2-phenyl-1-benzofuran-7-yl)methyl]-5-methyl-1H-pyrazol-3-amine (0.200 g, 0.6 mmol) in dry DCM (4.0 ml) was stirred at room temperature under an atmosphere of argon. EDAC (0.138 g, 0.72 mmol) and HOBt (0.096 g, 0.72 mmol) were added to the solution. (R)—N-(tert-Butoxycarbonyl)-piperidine-3-carboxylic acid (0.164 g, 0.72 mmol) was added to the solution and stirring continued at room temperature for 18 hours. The solution was diluted with DCM, and washed with water. The solution was dried over Na₂SO₄, and the organics concentrated under reduced pressure. The residue was chromatographed [SiO₂, Hexane/EtOAc, 50-100%] to give the title compound. (0.211 g)

LC/MS Rt=3.82 min [M+H]⁺ 549, 551

The following compounds were prepared in a similar manner from 1-[(5-chloro-2-phenyl-1-benzofuran-7-yl)methyl]-5-methyl-1H-pyrazol-3-amine (0.200 g, 0.6 mmol) and the appropriate acid. In some cases, it was necessary to purify the compounds using MDAP.

| | | | LC-MS |
|---|---|---|---|
| Description | Structure | Name | Data |
| 106 (D106) | 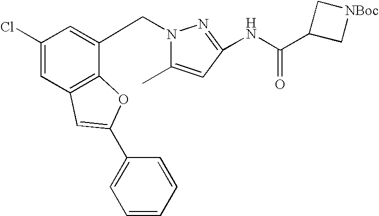 | 1,1-dimethylethyl 3-[({1- [(5-chloro-2-phenyl-1- benzofuran-7-yl)methyl]- 5-methyl-1H-pyrazol-3- yl}amino)carbonyl]-1- azetidinecarboxylate | Rt = 3.64 min [M + H]⁺ 521, 523 |
| 107 (D107) | 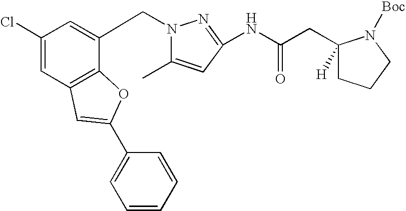 | 1,1-dimethylethyl (2S)-2- [2-({1-[(5-chloro-2- phenyl-1-benzofuran-7- yl)methyl]-5-methyl-1H- pyrazol-3-yl}amino)-2- oxoethyl]-1- pyrrolidinecarboxylate | Rt = 3.78 min [M + H]⁺ 549, 551 |

### Example 22 (E22)

### 1-[(5-Chloro-2-phenyl-1-benzofuran-7-yl)methyl]-5-methyl-N-[5-(1-piperazinylmethyl)-2-pyridinyl]-1H-pyrazole-3-carboxamide hydrochloride

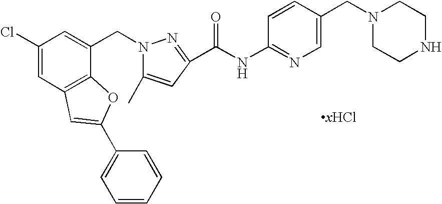

1-[(5-chloro-2-phenyl-1-benzofuran-7-yl)methyl]-N-(5-formyl-2-pyridinyl)-5-methyl-1H-pyrazole-3-carboxamide (56 mg, 0.119 mmol) was stirred in dry DCM (0.3 ml). 1-Boc-piperazine (27 mg, 0.143 mmol) was added and the reaction mixture was stirred at room temperature for 20 min. NaBH(OAc)₃ was added to the mixture. The mixture was then stirred at room temperature under argon for 40 min. 1 spatula of MS added and the mixture stirred at room temperature under an atmosphere of argon for a further 40 min. The reaction mixture was diluted with DCM. The organic layer was washed with 2M NaOH (2 ml). The organic layer was removed and the aqueous layer washed with further DCM (×2). The combined organics were washed with brine. The organics were dried over MgSO₄, filtered and concentrated to give a colourless oil. The residue was chromatographed [SiO₂, 50-70% EtOAc in Hexane]. The product was then stirred in 4M HCl in dioxane for 30 min. The reaction mixture was concentrated under reduced pressure to give the title compounds as a white solid. The solid was washed with diethyl ether. (33 mg)

LC/MS Rt=2.43 min [M+H]⁺ 541, 543

### Example 23 (E23)

### 1-[(5-Chloro-2-phenyl-1-benzofuran-7-yl)methyl]-N-{4-[(ethylamino)methyl]phenyl}-5-methyl-1H-pyrazole-3-carboxamide hydrochloride

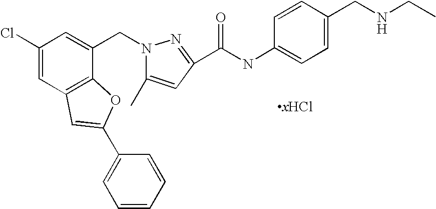

1-[(5-chloro-2-phenyl-1-benzofuran-7-yl)methyl]-N-(4-formylphenyl)-5-methyl-1H-pyrazole-3-carboxamide (200 mg, 0.426 mmol) was dissolved in dry DCM (4 ml). Ethylamine (0.256 ml) was added and the solution was stirred at room temperature under an atmosphere of argon for 30 minutes. After this time, acetic acid (0.1 ml) and NaBH(OAc)₃ (108 mg, 0.511 mmol) were added. The solution was stirred at room temperature under argon for 3 hours. A further 1.2 equiv. of ethylamine, 1.2 equiv of NaBH(OAc)₃ and a spatula of 4 Å MS (powdered) were added, and the solution left to stir at room temperature under an atmosphere of argon overnight (17 hours). Reaction mixture was diluted with DCM, and the organics washed with 2M NaOH (2 ml). The aqueous layer was washed with further DCM (×2). Brine was added to encourage separation. Combined organics were dried over MgSO₄, filtered and concentrated. The residue was chromatographed [SiO₂, 75% EtOAc in Hexane then 1% ammonia in 75% EtOAc in Hexane] to give pure product. The product was then treated with 1M HCl in Et₂O at room temperature for 30 minutes and concentrated to give the title compound. (151 mg)

Rt=2.78 min [M+H]⁺ 499, 501.

The following compounds were prepared in a similar manner using the appropriate aldehyde and amine, in either DCM or THF as solvent, purifying by chromatography or trituration with ether. If the HCl salt was required the compound was treated with 1M HCl in Et₂O.

| Example no. | Structure | Name | LC/MS Data |
|---|---|---|---|
| 24(E24) | 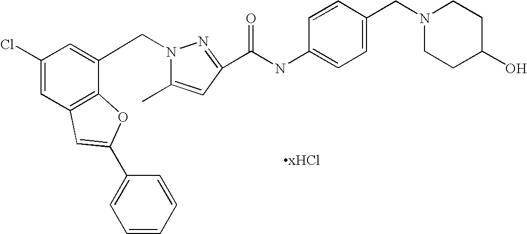 | 1-[(5-Chloro-2-phenyl-1- benzofuran-7-yl)methyl]- N-{4-[(4-hydroxy-1- piperidinyl)methyl]phenyl}- 5-methyl-1H-pyrazole-3- carboxamide | Rt = 2.74 min [M + H]⁺ 555, 557 |
| 25(E25) | 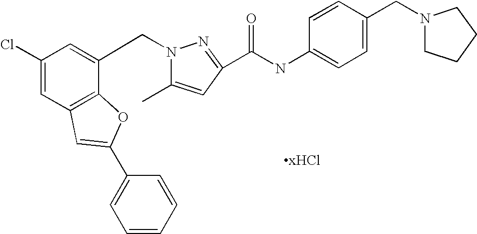 | 1-[(5-Chloro-2-phenyl-1- benzofuran-7-yl)methyl]- 5-methyl-N-[4-(1- pyrrolidinylmethyl)phenyl]- 1H-pyrazole-3- carboxamide | Rt = 2.77 min [M + H]⁺ 525, 527 |
| 26(E26) | 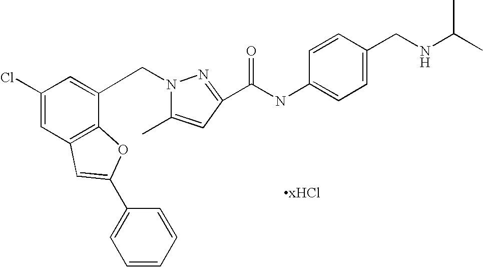 | 1-[(5-Chloro-2-phenyl-1- benzofuran-7-yl)methyl]- 5-methyl-N-(4-{[(1- methylethyl)amino]methyl} phenyl)-1H-pyrazole-3- carboxamide | Rt = 1.88 min [MH⁺] 513, 515 |
| 27 (E27) | 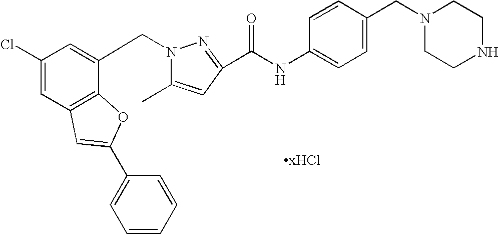 | 1-[(5-Chloro-2-phenyl-1- benzofuran-7-yl)methyl]- 5-methyl-N-[4-(1- piperazinyl)phenyl]-1H- pyrazole-3-carboxamide | |
| 28 (E28) | 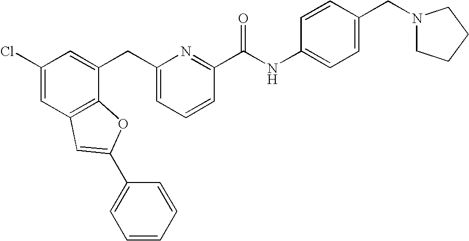 | 6-[(5-Chloro-2-phenyl-1- benzofuran-7-yl)methyl]- N-[4-(1- pyrrolidinylmethyl)phenyl]- 2-pyridinecarboxamide | Rt = 2.94 min |
| 29 (E29) | 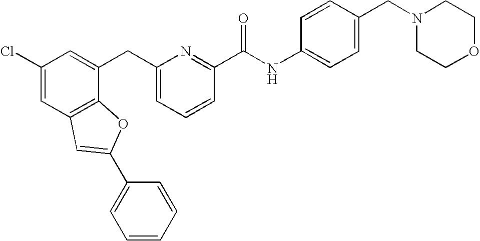 | 6-[(5-Chloro-2-phenyl-1- benzofuran-7-yl)methyl]- N-[4-(4- morpholinylmethyl) phenyl]- 2-pyridinecarboxamide | Rt = 2.87 min |

### Example 30 (E30)

### 1-{[5-chloro-2-(2,4-dichlorophenyl)-1-benzofuran-7-yl]methyl}-5-methyl-N-(4-piperidinylmethyl)-1H-pyrazole-3-carboxamide hydrochloride

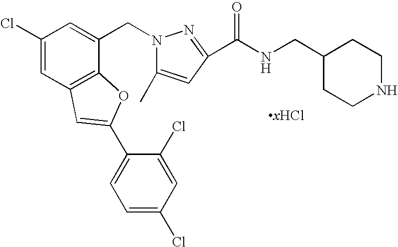

Solution of 1,1-dimethylethyl 4-({[(1-{[5-chloro-2-(2,4-dichlorophenyl)-1-benzofuran-7-yl]methyl}-5-methyl-1H-pyrazol-3-yl)carbonyl]amino}methyl)-1-piperidinecarboxylate (0.31 mmol) in HCl in Et₂O (1.0 ml, 1M) was stirred at room temperature under an atmosphere of argon overnight. LC/MS showed removal of the Boc group. Solvent was removed under reduced pressure to give an off-white solid (0.102 g).

LC/MS Rt=2.60 min [M+H]⁺ 533, 535.

The following compound was prepared in a similar manner from the appropriate amines.

| Example no. | Structure | Name | LC/MS Data |
|---|---|---|---|
| 31 (E31) | 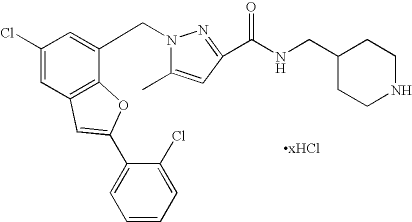 | 1-{[5-Chloro-2-(2- chlorophenyl)-1- benzofuran-7- yl]methyl}-5-methyl-N- (4-piperidinylmethyl)- 1H-pyrazole-3- carboxamide hydrochloride | Rt = 2.55 mins [M + H]⁺ 497, 500 |

### Example 32 (E32)

### (3R)—N-{1-[(5-chloro-2-phenyl-1-benzofuran-7-yl)methyl]-5-methyl-1H-pyrazol-3-yl}-3-piperidinecarboxamide methanesulfonate

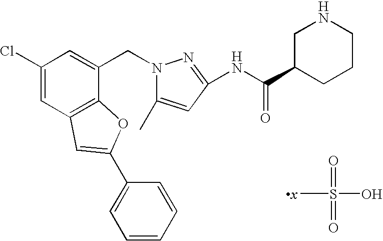

Solution of 1,1-dimethylethyl (3R)-3-[({1-[(5-chloro-2-phenyl-1-benzofuran-7-yl)methyl]-5-methyl-1H-pyrazol-3-yl}amino)carbonyl]-1-piperidinecarboxylate (0.211 g, 0.38 mmol) in dry DCM (0.5 ml) was stirred at room temperature. Trifluoroacetic acid (0.1 ml) was added and the solution stirred for 2 hours. After this time, the solvent was removed under reduced pressure to give a yellow coloured oil. Oil was dissolved in DCM and K₂CO₃ added. The inorganics were filtered off and MsOH was added to the solution. The solvent was then removed under reduced pressure and the resulting solid triturated with Et₂O to give the title compound (0.136 g)

The following compounds were prepared in a similar manner,

| | | | LC/MS |
|---|---|---|---|
| Example no. | Structure | Name | Data |
| 33 (E33) | 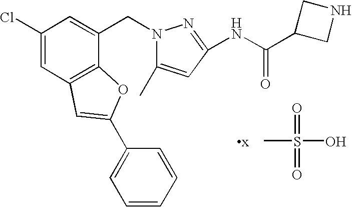 | N-{1-[(5-Chloro-2- phenyl-1-benzofuran-7- yl)methyl]-5-methyl-1H- pyrazol-3-yl}-3- azetidinecarboxamide methanesulfonate | Rt = 2.50 min, [M + H]⁺ 421, 423 |
| 34 (E34) | 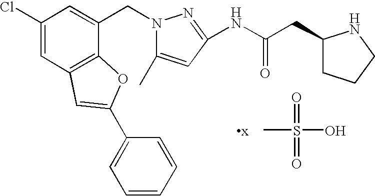 | N-{1-[(5-Chloro-2- phenyl-1-benzofuran-7- yl)methyl]-5-methyl-1H- pyrazol-3-yl}-2-[(2R)-2- pyrrolidinyl]acetamide methanesulfonate | Rt = 2.50 min [M + H]⁺ 449, 451 |

### Example 35 (E35)

### 5-Methyl-1-{[5-(methylsulfonyl)-2-phenyl-1-benzofuran-7-yl]methyl}-N-(4-piperidinylmethyl)-1H-pyrazole-3-carboxamide hydrochloride

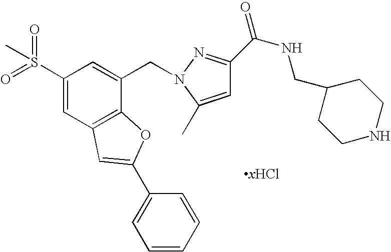

A solution of 1,1-dimethylethyl 4-({[(5-methyl-1-{[5-(methylsulfonyl)-2-phenyl-1-benzofuran-7-yl]methyl}-1H-pyrazol-3-yl)carbonyl]amino}methyl)-1-piperidinecarboxylate (250 mg, 0.41 mmol) in 4M hydrogen chloride in dioxan (5 ml) was stirred for about one minute during which time a gum separated preventing further stirring. After one hour the solvent was removed by evaporation and the residue dissolved in methanol (20 ml) and re-evaporated. The residue was triturated with ether to give the title compound as a white solid (198 mg).

LC/MS: Rt=2.05 min, [M+H]⁺ 507.2

The following compounds were prepared in a similar manner

| | | | LC/MS |
|---|---|---|---|
| Example no. | Structure | Name | data |
| 36 (E36) | 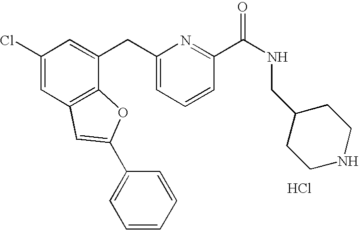 | 6-[(5-Chloro-2- phenyl-1-benzofuran- 7-yl)methyl]-N-(4- piperidinylmethyl)-2- pyridinecarboxamide hydrochloride | Rt = 2.44 min [M + H]⁺ 460.2 |
| 37 (E37) | 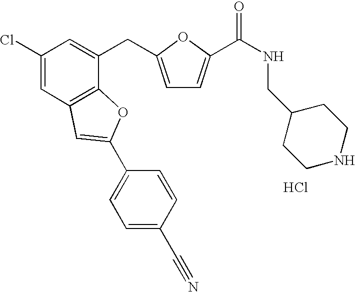 | 5-{[5-Chloro-2-(4- cyanophenyl)-1- benzofuran-7- yl]methyl}-N-(4- piperidinylmethyl)-2- furancarboxamide hydrochloride | Rt = 2.47 min [M + H]⁺ 474.2 |
| 38 (E38) | 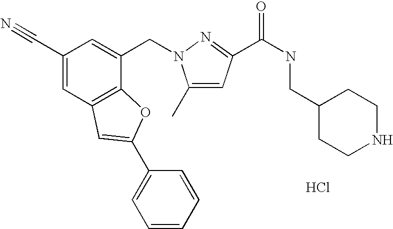 | 1-[(5-Cyano-2-phenyl- 1-benzofuran-7- yl)methyl]-5-methyl- N-(4- piperidinylmethyl)- 1H-pyrazole-3- carboxamide hydrochloride | Rt = 2.18 min [M + H]⁺ 454.2 |

### Example 39 (E39)

### 5-[(5-Chloro-2-phenyl-1-benzofuran-7-yl)methyl]-N-(4-piperidinylmethyl)-2-furancarboxamide hydrochloride

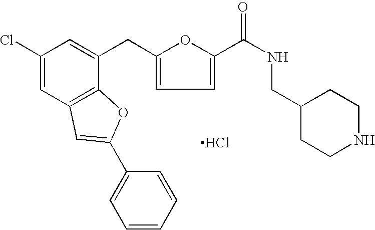

1,1-Dimethylethyl 4-{[({5-[(5-chloro-2-phenyl-1-benzofuran-7-yl)methyl]-2-furanyl}carbonyl)amino]methyl}-1-piperidinecarboxylate (110 mg) was dissolved in 4M hydrogen chloride in dioxan (4 ml) and left at room temperature for one hour. The solvent was evaporated and the residue dissolved in ethyl acetate (30 ml) and 2M sodium hydroxide (20 ml). The organic phase was separated and extracted with 2M hydrochloric acid (3×15 ml) and the combined acid extracts basified with 12.5M sodium hydroxide before being extracted with ethyl acetate (40 ml). The organic phase was dried (magnesium sulphate), evaporated and the residue dissolved in dichloromethane (5 ml) and treated with 1M hydrogen chloride in ether. The solvent was removed by evaporation and the residue triturated with ether to give the title compound as an off-white solid (26 mg).

LC/MS: Rt=2.42 min, [M+H]⁺ 449.1, 451.1

### Example 40 (E40)

### 1-{[5-Chloro-2-(2,4-difluorophenyl)-1-benzofuran-7-yl]methyl}-5-methyl-N-[4-(1-piperazinylmethyl)phenyl]-1H-pyrazole-3-carboxamide

### Hydrochloride Salt

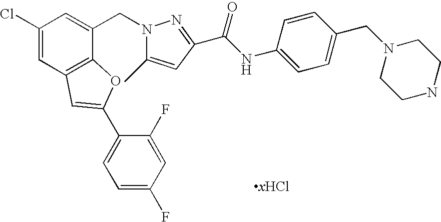

A solution of 1,1-dimethylethyl 4-[(4-{[(1-{[5-chloro-2-(2,4-difluorophenyl)-1-benzofuran-7-yl]methyl}-5-methyl-1H-pyrazol-3-yl)carbonyl]amino}phenyl)methyl]-1-piperazinecarboxylate (213 mg) in dichloromethane was treated with a solution of 4N hydrochloric acid in 1,4-dioxane (2 ml). After five minutes a precipitate formed and after thirty minutes the mixture was diluted with diethyl ether and the solid filtered off and washed with diethyl ether. The solid was purified by MDAP and then suspended in dichloromethane and treated with 1M hydrochloric acid in diethyl ether. The mixture was evaporated and diethyl ether added, then the solid was filtered off, washed with diethyl ether and dried at 60° C. under vacuum to afford the title compound as a pale green solid (113 mg).

LC/MS Rt=2.38 min. Molecular ion observed [M+H]⁺ 576, consistent with molecular formula C₃₁H₂₈N₅O₂³⁵ ClF₂

### Example 41 (E41)

### 1-{[5-Chloro-2-(2,4-difluorophenyl)-1-benzofuran-7-yl]methyl}-5-methyl-N-(4-piperidinylmethyl)-1H-pyrazole-3-carboxamide

### Hydrochloride Salt

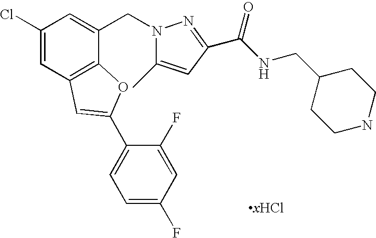

1,1-dimethylethyl 4-({[(1-{[5-chloro-2-(2,4-difluorophenyl)-1-benzofuran-7-yl]methyl}-5-methyl-1H-pyrazol-3-yl)carbonyl]amino}methyl)-1-piperidinecarboxylate (110 mg) was treated with a solution of 4N hydrochloric acid in 1,4-dioxane (4 ml). After five minutes a precipitate formed and after sixty minutes the mixture was diluted with diethyl ether and the solid filtered off and washed with diethyl ether then dried at 50° C. under vacuum to afford the title compound (81 mg).

LC/MS Rt=2.36 min. Molecular ion observed [M+H]⁺ 499, consistent with molecular formula C₂₆H₂₅N₄O₂³⁵ ClF₂

### Example 42 (E42)

### 1-{[5-Chloro-2-(4-chlorophenyl)-1-benzofuran-7-yl]methyl}-5-methyl-N-(4-piperidinylmethyl)-1H-pyrazole-3-carboxamide

### Hydrochloride Salt

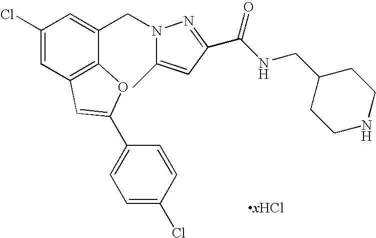

1,1-dimethylethyl 4-({[(1-{[5-chloro-2-(4-chlorophenyl)-1-benzofuran-7-yl]methyl}-5-methyl-1H-pyrazol-3-yl)carbonyl]amino}methyl)-1-piperidinecarboxylate (68 mg) was treated with a solution of 4N hydrochloric acid in 1,4-dioxane (2 ml). After one minute a precipitate formed and after thirty minutes the mixture was diluted with diethyl ether and the solid filtered off and washed with diethyl ether then dried at 50° C. under vacuum to afford the title compound as an off-white solid (40 mg).

LC/MS Rt=2.39 min. Molecular ion observed [M+H]⁺ 497, consistent with molecular formula C₂₆H₂₆N₄O₂³⁵Cl₂

### Example 43 (E43)

### 1-{[5-Chloro-2-(4-cyanophenyl)-1-benzofuran-7-yl]methyl}-5-methyl-N-(4-piperidinylmethyl)-1H-pyrazole-3-carboxamide

### Hydrochloride Salt

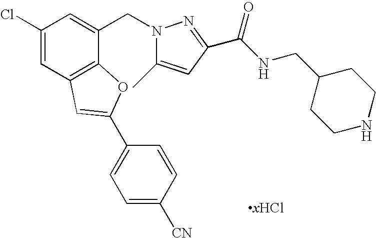

1,1-dimethylethyl 4-({[(1-{[5-chloro-2-(4-cyanophenyl)-1-benzofuran-7-yl]methyl}-5-methyl-1H-pyrazol-3-yl)carbonyl]amino}methyl)-1-piperidinecarboxylate was treated with a solution of 4N hydrochloric acid in 1,4-dioxane (6 ml). After thirty minutes the mixture was diluted with diethyl ether and the solid filtered off and washed with diethyl ether then dried at 50° C. under vacuum to afford the title compound (102 mg).

LC/MS Rt=2.21 min. Molecular ion observed [M+H]⁺ 488, consistent with molecular formula C₂₇H₂₆N₅O₂³⁵Cl

### Example 44 (E44)

### 6-[({1-[(5-Chloro-2-phenyl-1-benzofuran-7-yl)methyl]-5-methyl-1H-pyrazol-3-yl}carbonyl)amino]-N-(4-piperidinylmethyl)-3-pyridinecarboxamide

### Hydrochloride Salt

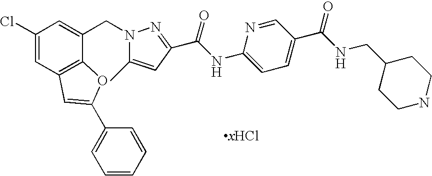

1,1-Dimethylethyl 4-{[({6-[({1-[(5-chloro-2-phenyl-1-benzofuran-7-yl)methyl]-5-methyl-1H-pyrazol-3-yl}carbonyl)amino]-3-pyridinyl}carbonyl)amino]methyl}-1-piperidinecarboxylate (62 mg) was treated with a solution of 4N hydrochloric acid in 1,4-dioxane (2 ml). After ninety minutes the mixture was diluted with diethyl ether and the mixture evaporated and then dried at 50° C. under vacuum to afford the title compound as a cream coloured solid (43 mg).

LC/MS Rt=2.47 min. Molecular ion observed [M+H]⁺ 583, consistent with molecular formula C₃₂H₃₁N₆O₃³⁵Cl

### Example 45 (E45)

### 1-[(5-Chloro-2-phenyl-1-benzofuran-7-yl)methyl]-5-methyl-N-[(3R)-3-pyrrolidinyl]-1H-pyrazole-3-carboxamide

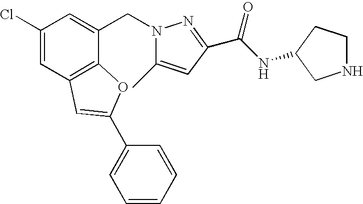

1,1-Dimethylethyl (3R)-3-[({1-[(5-chloro-2-phenyl-1-benzofuran-7-yl)methyl]-5-methyl-1H-pyrazol-3-yl}carbonyl)amino]-1-pyrrolidinecarboxylate (250 mg) was stirred in a solution of 4N hydrochloric acid in 1,4-dioxane (4 ml). The solid was filtered off and washed with saturated sodium bicarbonate, water and diethyl ether then dried at 45° C. under vacuum to afford the title compound (157 mg).

LC/MS Rt=2.33 min. Molecular ion observed [M+H]⁺ 435, consistent with molecular formula C₂₄H₂₃N₄O₂³⁵Cl

### Example 46 (E46)

### 1-[(5-chloro-2-phenyl-1-benzofuran-7-yl)methyl]-5-methyl-N-(4-piperidinylmethyl)-1H-pyrazole-3-carboxamide hydrochloride

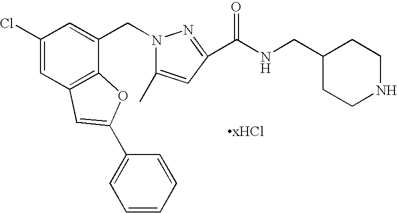

1-[(5-chloro-2-phenyl-1-benzofuran-7-yl)methyl]-5-methyl-1H-pyrazole-3-carbonyl chloride (400 mg, 1.038 mmol) was dissolved in dry DCM (2 ml) and DMF (1 ml). 4-(aminomethyl)-1—N-Boc piperidine (267 mg) and Et₃N (0.346 ml) were added to the solution. The solution was stirred at room temperature under an atmosphere of argon for 17 hours (overnight). The reaction mixture was then diluted with EtOAc and washed with sat. soln. NaHCO₃. Organics were separated and the aqueous layer was washed with further EtOAc. The organics were combined and washed with water (×2). Organics were then dried over MgSO₄, filtered and concentrated to give an oil. The oil was chromatographed [SiO₂, 40-75% EtOAc in Hexane]. The purified residue was then treated with 1M HCl in Et₂O for 2 hours to remove the Boc group.

LC/MS Rt=2.57 min, [M+H]⁺ 463, 465

The following compounds were prepared in a similar manner from the appropriate acid chloride and amine;

| Example no. | Structure | Name | Data |
|---|---|---|---|
| 47 (E47) | 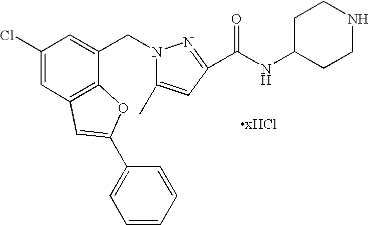 | 1-[(5-Chloro-2-phenyl-1- benzofuran-7-yl)methyl]- 5-methyl-N-4-piperidinyl- 1H-pyrazole-3- carboxamide | LC/MS Rt = 3.37 min [M + H]⁺ 485, 488 |
| 48 (E48) | 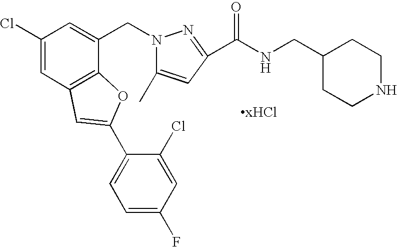 | 1-{[5-Chloro-2-(2-chloro- 4-fluorophenyl)-1- benzofuran-7-yl]methyl}- 5-methyl-N-(4- piperidinylmethyl)-1H- pyrazole-3-carboxamide hydrochloride | LC/MS Rt = 2.45 min [M + H]⁺ 515, 518 |

### Example 49 (E49)

### (2S)—N-{(2Z)-3-[({5-Chloro-2-[(1E,3Z)-1-ethenyl-1,3-pentadien-1-yl]-1-benzofuran-7-yl}methyl)(methyl)amino]-1-methylidene-2-buten-1-yl}-2-piperidinecarboxamide hydrochloride

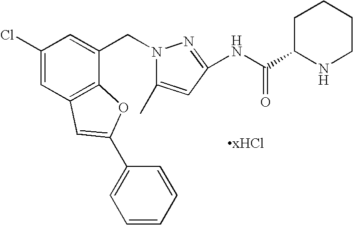

1-[(5-chloro-2-phenyl-1-benzofuran-7-yl)methyl]-5-methyl-1H-pyrazol-3-amine (200 mg, 0.593 mmol), EDAC (136 mg, 0.712 mmol) and HOBt (98 mg, 0.711 mmol) were stirred in dry DMF (2.0 ml). Boc-L-pipecolic acid (163 mg, 0.711 mmol) was added and the reaction mixture was stirred at room temperature under an atmosphere of argon for a total of 19 hours. The reaction mixture was then diluted with EtOAc and washed with NaHCO₃ (sat. aq. soln.). Organics were washed with further water (×3). The organics were dried over MgSO₄, filtered and concentrated. The residue was chromatographed [SiO₂, 20-50% EtOAc in Hexane]. The resulting product was treated with 4M HCl in dioxane for 10 min. A white solid formed which was filtered off and washed with ether. (163 mg)

The following compounds were prepared in a similar manner using the appropriate acid;

| | | | LC/MS |
|---|---|---|---|
| Example no. | Structure | Name | Data |
| 50 (E50) | 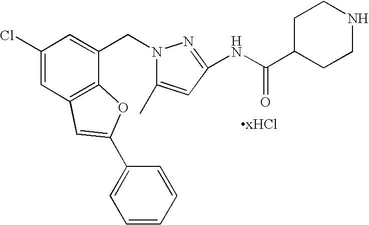 | N-{1-[(5-Chloro-2-phenyl- 1-benzofuran-7- yl)methyl]-5-methyl-1H- pyrazol-3-yl}-4- piperidinecarboxamide hydrochloride | |
| 51 (E51) | 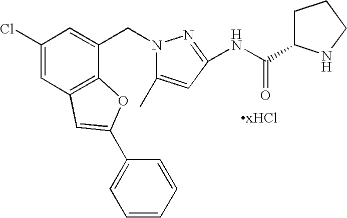 | N-{1-[(5-Chloro-2-phenyl- 1-benzofuran-7- yl)methyl]-5-methyl-1H- pyrazol-3-yl}-L- prolinamide hydrochloride | |
| 52 (E52) | 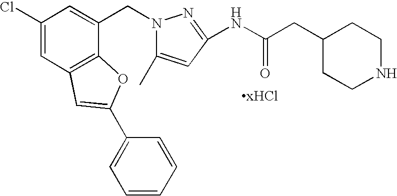 | N-{1-[(5-Chloro-2-phenyl- 1-benzofuran-7- yl)methyl]-5-methyl-1H- pyrazol-3-yl}-2-(4- piperidinyl)acetamide | LC/MS Rt = 2.31 min [M + H]⁺ 463, 465 |

### Example 53 (E53)

### N-{1-[(5-Chloro-2-phenyl-1-benzofuran-7-yl)methyl]-5-methyl-1H-pyrazol-3-yl}-4-fluoro-4-piperidinecarboxamide hydrochloride

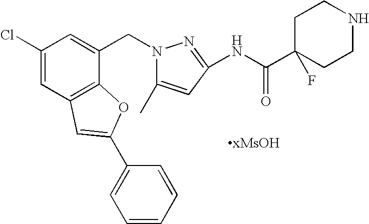

Solution of 1-[(5-chloro-2-phenyl-1-benzofuran-7-yl)methyl]-5-methyl-1H-pyrazol-3-amine (0.050 g, 0.15 mmol) in dry DCM (1.0 ml) was stirred at room temperature under argon. 1-{[(1,1-dimethylethyl)oxy]carbonyl}-4-fluoro-4-piperidinecarboxylic acid (0.037 g, 0.15 mmol), EDAC (0.028 g, 0.15 mmol) and HOBt (0.020 g, 0.15 mmol) were added and the solution stirred at room temperature overnight. After this time, the solution was diluted with DCM and the organics were washed with water. Organics were dried over MgSO₄, filtered and concentrated under reduced pressure to give a pale yellow oil. The residue was purified [SiO₂, EtOAc:Hex 50-100%] to give the title compound. The compound was further purified using MDAP and the sample treated with MsOH to remove the Boc group. (12 mg)

LC/MS Rt=2.56 min, [M+H]⁺ 467, 469.

### Example 54 (E54)

### 1-Acetyl-N-{1-[(5-chloro-2-phenyl-1-benzofuran-7-yl)methyl]-5-methyl-1H-pyrazol-3-yl}-3-azetidinecarboxamide

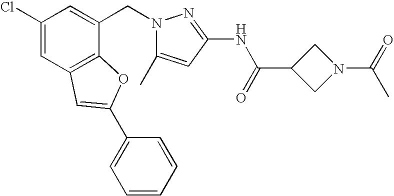

To a solution of N-{1-[(5-chloro-2-phenyl-1-benzofuran-7-yl)methyl]-5-methyl-1H-pyrazol-3-yl}-3-azetidinecarboxamide (83 mg) in dichloromethane (5 ml) at 0° C. under argon was added N-ethylmorpholine (0.1 ml) and acetic anhydride (0.026 ml). The reaction mixture was stirred for thirty minutes the ice-bath removed and stirred for a further thirty minutes. The reaction mixture was diluted with dichloromethane (20 ml) and washed with water (2×20 ml) then dried (MgSO₄) and evaporated. The residue was dissolved in dichloromethane and applied to a Biotage Si 12+S column and purified using the Biotage SP4 (gradient method) to afford the title compound (30 mg).

LC/MS Rt=3.04 min. Molecular ion observed [M+H]⁺ 463, consistent with molecular formula C₂₅H₂₃N₄O₃³⁵Cl

### Example 55 (E55)

### 2-[(2S)-1-Acetyl-2-pyrrolidinyl]-N-{1-[(5-chloro-2-phenyl-1-benzofuran-7-yl)methyl]-5-methyl-1H-pyrazol-3-yl}acetamide

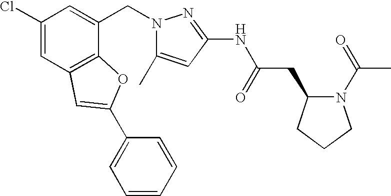

Prepared in a similar manner to 1-acetyl-N-{1-[(5-chloro-2-phenyl-1-benzofuran-7-yl)methyl]-5-methyl-1H-pyrazol-3-yl}-3-azetidinecarboxamide using N-{1-[(5-chloro-2-phenyl-1-benzofuran-7-yl)methyl]-5-methyl-1H-pyrazol-3-yl}-2-[(2S)-2-pyrrolidinyl]acetamide except that the title compound (41 mg) was not purified by chromatography.

LC/MS Rt=3.22 min. Molecular ion observed [M+H]⁺ 491, consistent with molecular formula C₂₇H₂₇N₄O₃³⁵Cl

### Example 56 (E56)

### N-[(3R)-1-Acetyl-3-pyrrolidinyl]-1-[(5-chloro-2-phenyl-1-benzofuran-7-yl)methyl]-5-methyl-1H-pyrazole-3-carboxamide

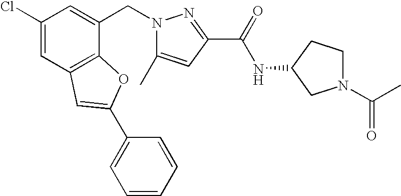

To a solution of 1-[(5-chloro-2-phenyl-1-benzofuran-7-yl)methyl]-5-methyl-N-[(3R)-3-pyrrolidinyl]-1H-pyrazole-3-carboxamide (40 mg) in dichloromethane (5 ml) at 0° C. under argon was added N-ethylmorpholine (0.051 ml) and acetic anhydride (0.013 ml). The reaction mixture was stirred for thirty minutes the ice-bath removed and stirred for a further thirty minutes. The reaction mixture was diluted with dichloromethane (20 ml) and washed with water (2×20 ml) then dried (MgSO₄) and evaporated to afford the title compound (27 mg).

LC/MS Rt=3.07 min. Molecular ion observed [M+H]⁺ 477, consistent with molecular formula C₂₆H₂₅N₄O₃³⁵Cl

### Example 57 (E57)

### 1-[(5-chloro-2-phenyl-1-benzofuran-7-yl)methyl]-N-[(1-ethyl-4-piperidinyl)methyl]-5-methyl-1H-pyrazole-3-carboxamide

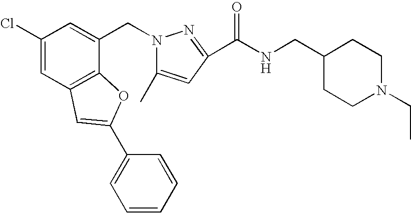

1-[(5-chloro-2-phenyl-1-benzofuran-7-yl)methyl]-5-methyl-N-(4-piperidinylmethyl)-1H-pyrazole-3-carboxamide hydrochloride (150 mg, 0.324 mmol) was dissolved in dry DCM (0.6 ml). Acetaldehyde (0.022 ml, 0.389 mmol) and AcOH (0.1 ml) were added. The solution turned orange/brown. The solution was stirred at room temperature under an atmosphere of argon for 30 min. NaBH(OAc)₃ (82 mg, 0.389 mmol) was added and the solution was stirred at room temperature for 72 hours. The reaction mixture was then diluted with DCM and washed with 2M NaOH (2 ml). Aqueous layer was removed and washed with further DCM (×2). Brine was added to encourage separation. The organics were combined, dried over MgSO₄, filtered and concentrated to give an orange oil. The residue was chromatographed [SiO₂, 75-100% EtOAc in Hexane, then 1% ammonia in hexane]. The sample was then purified further using MDAP to give the title compound. (33 mg)

LC/MS Rt=2.33 min [M+H]⁺ 491, 493.

### Example 58 (E58)

### N-{1-[(5-chloro-2-phenyl-1-benzofuran-7-yl)methyl]-5-methyl-1H-pyrazol-3-yl}-1-(1-methylethyl)-L-prolinamide hydrochloride

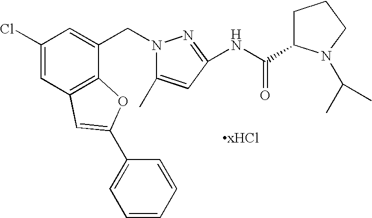

N-{1-[(5-chloro-2-phenyl-1-benzofuran-7-yl)methyl]-5-methyl-1H-pyrazol-3-yl}-L-prolinamide hydrochloride (132 mg, 0.280 mmol) was dissolved in DCM and DMF (minimum amount). 2M NaOH (2 ml) was added to the solution. Organics were extracted into DCM, then dried over MgSO₄, filtered and concentrated under reduced pressure. The resulting oil was dried, then dissolved in dry DCM (0.6 ml). 4 Å MS (2 spatulas) and acetone (0.031 ml, 0.420 mmol) were added to the solution. The solution was then stirred at room temperature under argon for 30 min. After this item, NaBH(OAc)₃ (89 mg, 0.420 mmol) was added and the reaction stirred for 1 hour. Further acetone (0.031 ml) and NaBH(OAc)₃ (89 mg) were added to the reaction. The reaction was stirred at room temperature for 1 hour. The reaction mixture was diluted with water and 2M NaOH (2 ml). The organics were extracted with DCM (×2). The combined organics were dried over MgSO₄, filtered and concentrated to give a white foam the residue was stirred in 1M HCl in dioxane (4 ml) for 20 min then concentrated under reduced pressure to give a cream coloured solid. (121 mg)

LC/MS Rt=2.42 min, [M+H]⁺ 477, 479.

### Example 59 (E59)

### 1-{[5-Chloro-2-(2,4-dichlorophenyl)-1-benzofuran-7-yl]methyl}-5-methyl-1H-pyrazole-3-carboxamide

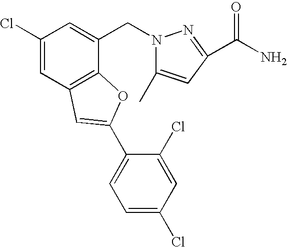

Solution of 1-{[5-chloro-2-(2,4-dichlorophenyl)-1-benzofuran-7-yl]methyl}-5-methyl-1H-pyrazole-3-carbonyl chloride (0.145 g, 0.31 mmol) in dry DCM (2.0 ml) was stirred at room temperature under an atmosphere of argon. Et₃N (0.086 ml, 0.62 mmol) was added to the solution. Ammonium hydroxide (0.2 ml, 0.46 mmol) was added to the mixture and stirring continued at room temperature overnight. After this time, solvent was removed under reduced pressure and the residue purified using MDAP to give the title compound. (0.012 g).

LC/MS Rt=3.73 min [M+H]⁺ 436.

### Example 60 (E60)

### 1-[(5-chloro-2-phenyl-1-benzofuran-7-yl)methyl]-5-methyl-N-4-morpholinyl-1H-pyrazole-3-carboxamide

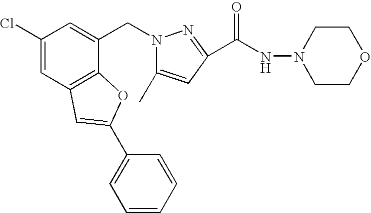

Solution of 1-[(5-Chloro-2-phenyl-1-benzofuran-7-yl)methyl]-5-methyl-1H-pyrazole-3-carboxylic acid (2.00 g, 5.46 mmol) in dry DMF (22 ml) was stirred at room temperature under an atmosphere of argon. 4-amino morpholine (0.630 ml, 6.55 mmol), EDAC hydrochloride (1.251 g, 6.55 mmol) and 1-hydroxybenzotriazole (0.884 g, 6.55 mmol) were added to the solution. The solution was stirred at room temperature for 3 hours. The mixture was then diluted with EtOAc and washed with water (×3). Organics were dried over MgSO₄, filtered and concentrated under reduced pressure to give a yellow coloured oil. The residue was chromatographed [SiO₂, EtOAc] to give the title compound. (1.196 g)

LC/MS Rt=3.13 min, [M+H]⁺ 451, 453.

¹H NMR (CDCl₃, 400 MHz) δ: 2.32 (3H, s); 2.94-2.96 (4H, m); 3.85-3.87 (4H, m); 5.59 (2H, s); 6.68 (1H, s); 6.82 (1H, s); 7.00 (1H, s); 7.49-7.43 (1H, m); 7.46-7.51 (3H, m); 7.62 (1H, s); 7.82 (2H, dd, J 8.4, 1.6 Hz)

The following compounds were prepared in a similar manner from the appropriate acids and 4-amino-morpholine, extracting the organics into either DCM or EtOAc.

| | | | LC/MS |
|---|---|---|---|
| Example no. | Structure | Name | Data |
| 61 (E61) | 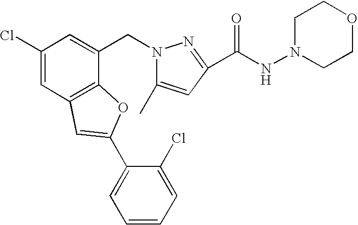 | 1-{[5-chloro-2-(2- chlorophenyl)-1- benzofuran-7-yl]methyl}- 5-methyl-N-4- morpholinyl-1H- pyrazole-3-carboxamide | LC/MS Rt = 3.37 min [M + H]⁺ 485, 488 |
| 62 (E62) | 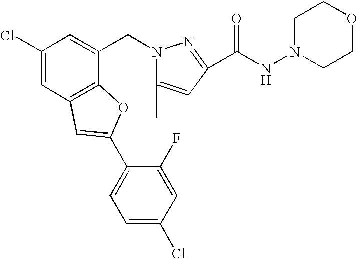 | 1-{[5-chloro-2-(4-chloro- 2-fluorophenyl)-1- benzofuran-7-yl]methyl}- 5-methyl-N-4- morpholinyl-1H- pyrazole-3-carboxamide | LC/MS Rt = 3.51 min [M + H]⁺ 503, 506 |
| 63 (E63) | 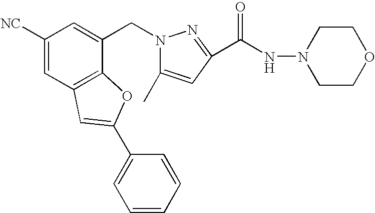 | 1-[(5-cyano-2-phenyl-1- benzofuran-7-yl)methyl]- 5-methyl-N-4- morpholinyl-1H- pyrazole-3-carboxamide | LC/MS Rt = 2.82 min, [MH⁺] 442, 443 |

### Example 64 (E64)

### 1-[(5-Chloro-2-phenyl-1-benzofuran-7-yl)methyl]-N-(1,1-dimethylethyl)-5-methyl-1H-pyrazole-3-carboxamide

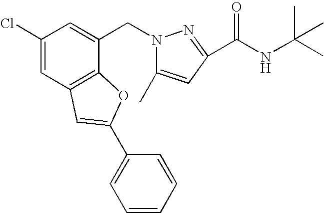

1-[(5-chloro-2-phenyl-1-benzofuran-7-yl)methyl]-5-methyl-1H-pyrazole-3-carbonyl chloride (150 mg, 0.390 mmol) was dissolved in dry DCM (1.6 ml) and DMF (1.6 ml). tert-butylamine (34 mg) and Et₃N (0.130 ml) were added. The solution was stirred at room temperature under an atmosphere of argon for 2 hours. The reaction mixture was then diluted with EtOAc and washed with sat. soln. of NaHCO₃. Organics were separated and the aqueous layer was washed with EtOAc (×2). The combined organics were dried over MgSO₄, filtered and concentrated under reduced pressure. The residue was chromatographed [SiO₂, 40-65% EtOAc in Hexane] to give a white solid. The solid was washed with hexane to give the title compound. (125 mg)

LC/MS Rt=3.68 min, [M+H]⁺ 422, 424

¹H NMR (CDCl₃, 400 MHz) δ: 1.47 (9H, s); 2.30 (3H, s); 5.59 (2H, s); 6.60 (1H, s); 6.75 (1H, bs); 6.81 (1H, s); 6.99 (1H, s); 7.38-7.42 (1H, m); 7.46-7.50 (3H, m); 7.82 (2H, d, J 8.8, 1.6 Hz)

The following compounds were prepared in a similar manner from the appropriate amine and acid chloride;

| | | | LC/MS |
|---|---|---|---|
| Example no. | Structure | Name | Data |
| 65 (E65) | 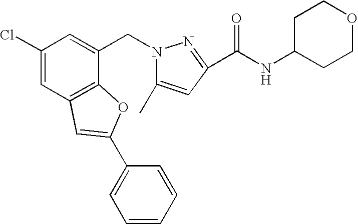 | 1-[(5-chloro-2-phenyl-1- benzofuran-7-yl)methyl]- 5-methyl-N-(tetrahydro- 2H-pyran-4-yl)-1H- pyrazole-3-carboxamide | Rt = 3.26 min, [M + H]⁺ 450, 452 |
| 66 (E66) | 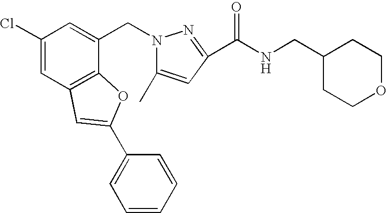 | 1-[(5-chloro-2-phenyl-1- benzofuran-7-yl)methyl]- 5-methyl-N-(tetrahydro- 2H-pyran-4-ylmethyl)-1H- pyrazole-3-carboxamide | Rt = 3.32 min [M + H]⁺ 464, 466 |
| 67 (E67) | 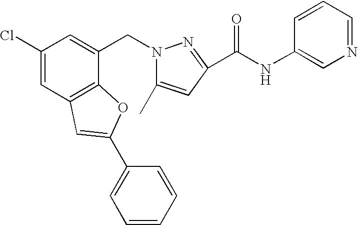 | 1-[(5-chloro-2-phenyl-1- benzofuran-7-yl)methyl]- 5-methyl-N-3-pyridinyl- 1H-pyrazole-3- carboxamide | Rt = 3.09 min [M + H]⁺ 443, 445 |
| 68 (E68) | 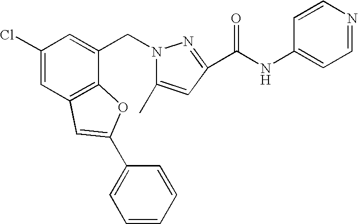 | 1-[(5-chloro-2-phenyl-1- benzofuran-7-yl)methyl]- 5-methyl-N-4-pyridinyl- 1H-pyrazole-3- carboxamide | Rt = 2.80 min [M + H]⁺ 443, 445 |
| 69 (E69) | 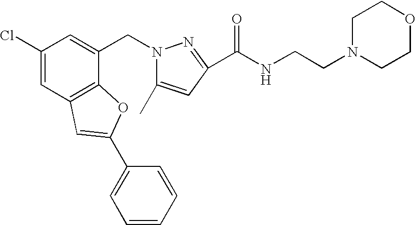 | 1-[(5-chloro-2-phenyl-1- benzofuran-7-yl)methyl]- 5-methyl-N-[2-(4- morpholinyl)ethyl]-1H- pyrazole-3-carboxamide | Rt = 2.26 min [M + H]⁺ 479, 481 |
| 70 (E70) | 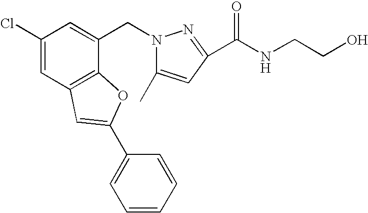 | 1-[(5-chloro-2-phenyl-1- benzofuran-7-yl)methyl]- N-(2-hydroxyethyl)-5- methyl-1H-pyrazole-3- carboxamide | Rt = 3.00 min [M + H]⁺ 410, 412 |
| 71 (E71) | 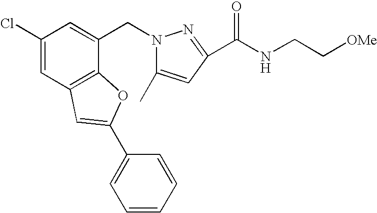 | 1-[(5-chloro-2-phenyl-1- benzofuran-7-yl)methyl]- 5-methyl-N-[2- (methyloxy)ethyl]-1H- pyrazole-3-carboxamide | Rt = 3.30 min [M + H]⁺ 424, 426 |
| 72 (E72) | 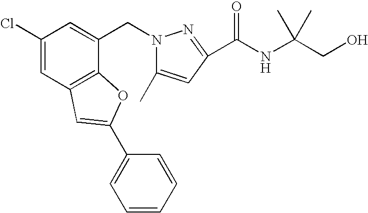 | 1-[(5-chloro-2-phenyl-1- benzofuran-7-yl)methyl]- N-(2-hydroxy-1,1- dimethylethyl)-5-methyl- 1H-pyrazole-3- carboxamide | Rt = 3.38 min, [M + H]⁺ 438, 440 |
| 73 (E73) | 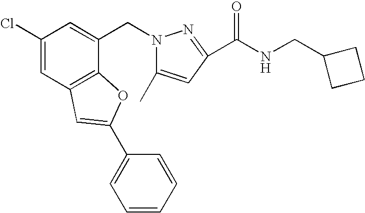 | 1-[(5-chloro-2-phenyl-1- benzofuran-7-yl)methyl]- N-(cyclobutylmethyl)-5- methyl-1H-pyrazole-3- carboxamide | Rt = 3.80 min, [M + H]⁺ 434, 436 |
| 74 (E74) | 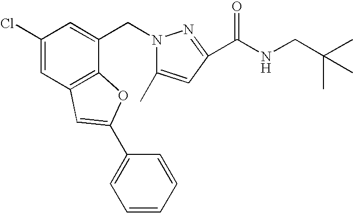 | 1-[(5-chloro-2-phenyl-1- benzofuran-7-yl)methyl]- N-(2,2-dimethylpropyl)-5- methyl-1H-pyrazole-3- carboxamide | Rt = 3.75 min, [M + H]⁺ 436, 438 |
| 75 (E75) | 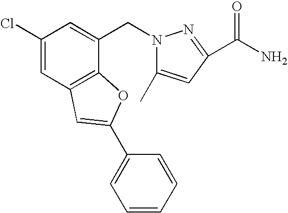 | 1-[(5-chloro-2-phenyl-1- benzofuran-7-yl)methyl]- 5-methyl-1H-pyrazole-3- carboxamide | Rt = 3.73 min, [M + H]⁺ 366, 368 |
| 76 (E76) | 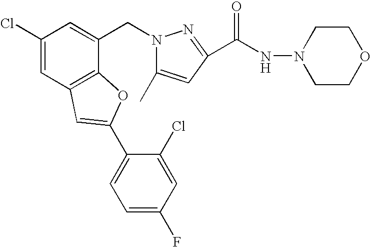 | 1-{[5-chloro-2-(2-chloro- 4-fluorophenyl)-1- benzofuran-7-yl]methyl}- 5-methyl-N-4- morpholinyl-1H- pyrazole-3-carboxamide | Rt = 3.34 min [M + H]⁺ 503, 505 |

### Example 77 (E77)

### 1-[(5-Chloro-2-phenyl-1-benzofuran-7-yl)methyl]-5-methyl-N-(1-methylethyl)-1H-pyrazole-3-carboxamide

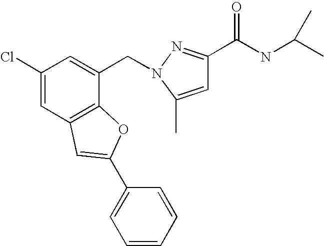

1-[(5-chloro-2-phenyl-1-benzofuran-7-yl)methyl]-5-methyl-1H-pyrazole-3-carboxylic acid (50 mg, 0.136 mmol), EDAC (31.5 mg. 0.164 mmol), HOBt (22 mg, 0.164 mmol) and 2-propanamine(14.4 μl, 0.164 mmol) in DCM (4 ml), were stirred at room temperature under argon for 2 hours. Diluted with more DCM and washed with a saturated solution of sodium bicarbonate in a phase separator cartridge. The organic phase was evaporated and the residue was purified on the SP4 using 50-90% of ethyl acetate in hexane. The residue was triturated with diethyl ether to give the title compound as white solid.

LC/MS Rt=3.62, [M+H]⁺ 408.1, 410.1

The following compounds were prepared in a similar manner to 1-[(5-Chloro-2-phenyl-1-benzofuran-7-yl)methyl]-5-methyl-N-(1-methylethyl)-1H-pyrazole-3-carboxamide.

| Example | Structure | Name | LC/MS data |
|---|---|---|---|
| 78 (E78) | 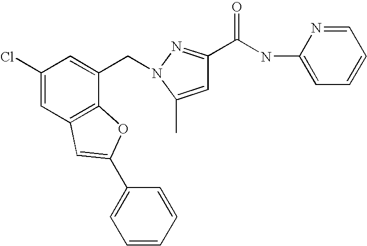 | 1-[(5-chloro-2-phenyl-1- benzofuran-7-yl)methyl]-5- methyl-N-2-pyridinyl-1H- pyrazole-3-carboxamide | Rt = 3.87, [M + H]⁺ 443.1, 445.1 |
| 79 (E79) | 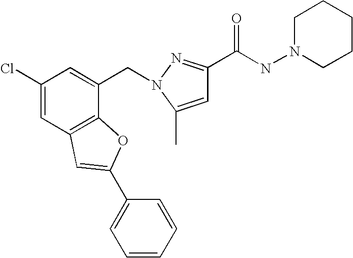 | 1-[(5-Chloro-2-phenyl-1- benzofuran-7-yl)methyl]-5- methyl-N-1-piperidinyl-1H- pyrazole-3-carboxamide | Rt = 3.51, [M + H]⁺ 449.1, 451.1, 452.1 |
| 80 (E80) | 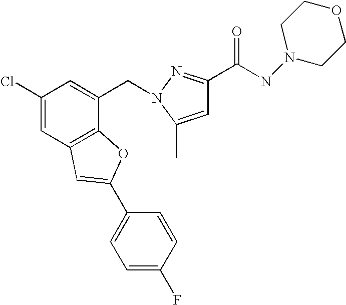 | 1-{[5-Chloro-2-(4- fluorophenyl)-1- benzofuran-7-yl]methyl}-5- methyl-N-4-morpholinyl- 1H-pyrazole-3- carboxamide | Rt = 3.13, [M + H]⁺ 469.1, 471.1 |

### Example 81 (E81)

### 1-{[5-Chloro-2-(3-pyridinyl)-1-benzofuran-7-yl]methyl}-5-methyl-N-(1-methylethyl)-1H-pyrazole-3-carboxamide

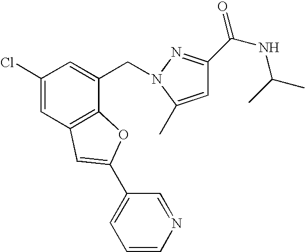

Oxalyl chloride (0.2 ml) was added to a suspension of 1-{[5-chloro-2-(3-pyridinyl)-1-benzofuran-7-yl]methyl}-5-methyl-1H-pyrazole-3-carboxylic acid (80 mg, 0.2 mmol) and DMF (1 drop) in dichloromethane (5 ml) producing a colourless solution which was left at room temperature for 30 minutes. The solution was evaporated and azeotroped with toluene (2 ml). The residue was dissolved in dichloromethane (10 ml) and isopropylamine (0.5 ml) added with stirring. After 30 minutes the solution was washed with water (10 ml), dried (magnesium sulphate), evaporated and purified by flash chromatography on a Biotage column eluting with ethyl acetate to give the title compound as a white solid (41 mg).

LC/MS: Rt=2.89 min, [M+H]⁺ 409.2, 411.1

The following compounds were prepared in a similar manner by reaction of the appropriate acid with oxalyl chloride then with isopropylamine or 0.88 aqueous ammonia or N,N-dimethylhydrazine or N,N-dimethylethylenediamine.

| Example | | | LC/MS |
|---|---|---|---|
| No. | Structure | Name | data |
| (E82) | 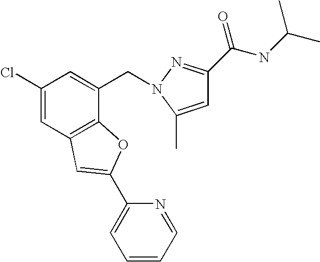 | 1-{[5-Chloro-2-(2- pyridinyl)-1-benzofuran- 7-yl]methyl}-5-methyl-N- (1-methylethyl)-1H- pyrazole-3-carboxamide | Rt = 3.10 min, [M + H]⁺ 409.2, 411.1 |
| 83 (E83) | 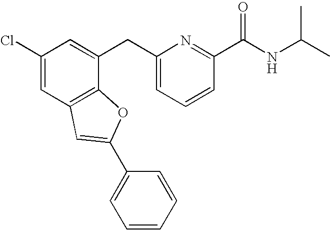 | 6-[(5-Chloro-2-phenyl-1- benzofuran-7- yl)methyl]-N-(1- methylethyl)-2- pyridinecarboxamide | Rt = 3.96 min, [M + H] 405.2, 407.2 |
| 84 (E84) | 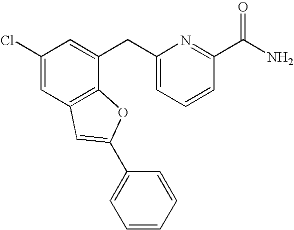 | 6-[(5-Chloro-2-phenyl-1- benzofuran-7- yl)methyl]-2- pyridinecarboxamide | Rt = 3.48 min, [M + H]⁺ 363.1, 365.1 |
| 85 (E85) | 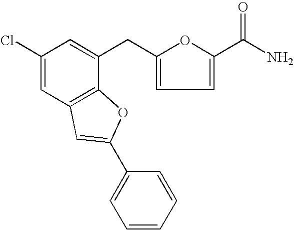 | 5-[(5-Chloro-2-phenyl-1- benzofuran-7- yl)methyl]-2- furancarboxamide | Rt = 3.27 min, [M + H]⁺ 352.1, 354.1 |
| 86 (E86) | 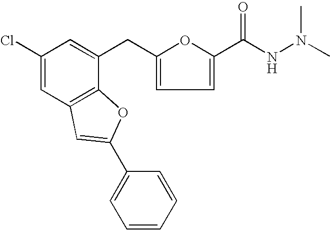 | 5-[(5-Chloro-2-phenyl-1- benzofuran-7- yl)methyl]-N′,N′- dimethyl-2- furancarbohydrazide | Rt = 3.31 min, [M + H]⁺ 395.1, 397.1 |
| 87 (E87) | 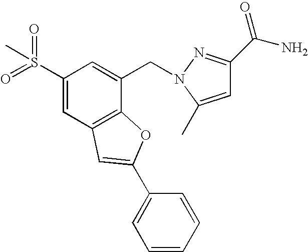 | 5-Methyl-1-{[5- (methylsulfonyl)-2- phenyl-1-benzofuran-7- yl]methyl}-1H-pyrazole- 3-carboxamide | Rt = 2.57 min, [M + H]⁺ 410.2 |
| 88 (E88) | 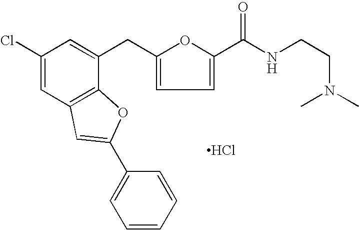 | 5-[(5-Chloro-2-phenyl-1- benzofuran-7- yl)methyl]-N-[2- (dimethylamino)ethyl]- 2-furancarboxamide hydrochloride | Rt = 2.43 min, [M + H]⁺ 423.2, 425.2 |
| 89 (E89) | 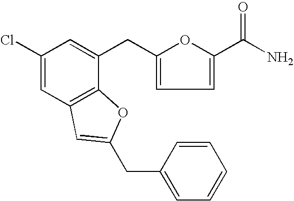 | 5-{[5-Chloro-2- (phenylmethyl)-1- benzofuran-7- yl]methyl}-2- furancarboxamide | Rt = 3.26 min, [M + H]⁺ 366.1, 368.1 |
| 90 (E90) | 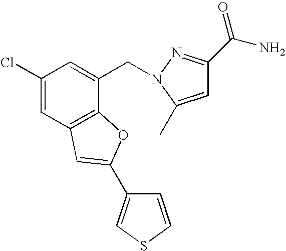 | 1-{[5-Chloro-2-(3- thienyl)-1-benzofuran-7- yl]methyl}-5-methyl-1H- pyrazole-3-carboxamide | Rt = 3.06 min, [M + H]⁺ 372.2, 374.1 |
| 91 (E91) | 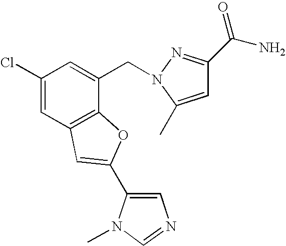 | 1-{[5-Chloro-2-(1- methyl-1H-imidazol-5- yl)-1-benzofuran-7- yl]methyl}-5-methyl-1H- pyrazole-3-carboxamide | Rt = 1.81 min, [M + H]⁺ 370.2, 372.2 |
| 92 (E92) | 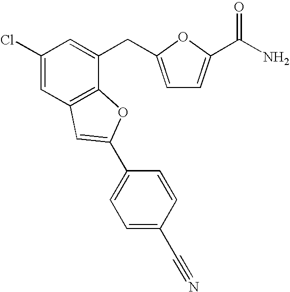 | 5-{[5-Chloro-2-(4- cyanophenyl)-1- benzofuran-7- yl]methyl}-2- furancarboxamide | Rt = 3.17 min [M + H]⁺ 377.1 |
| 93 (E93) | 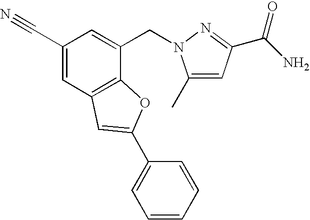 | 1-[(5-Cyano-2-phenyl-1- benzofuran-7- yl)methyl]-5-methyl-1H- pyrazole-3-carboxamide | Rt = 2.83 min [M + H]⁺ 357.2 |

### Example 94 (E94)

### 1-{[5-Chloro-2-(2-pyridinyl)-1-benzofuran-7-yl]methyl}-5-methyl-N-4-morpholinyl-1H-pyrazole-3-carboxamide

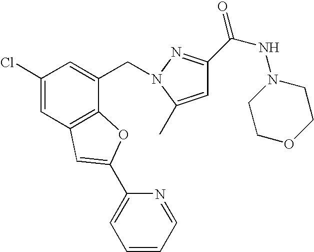

Oxalyl chloride (0.2 ml) was added to a suspension of 1-{[5-chloro-2-(2-pyridinyl)-1-benzofuran-7-yl]methyl}-5-methyl-1H-pyrazole-3-carboxylic acid (80 mg, 0.2 mmol) and DMF (1 drop) in dichloromethane (5 ml) and left at room temperature for 30 minutes. The solution was evaporated and azeotroped with toluene (5 ml). The residue was dissolved in dichloromethane (5 ml) and a solution of 4-aminomorpholine (51 mg, 0.5 mmol) in pyridine (0.3 ml) was added and stirred for one hour. The resulting solution was washed with water (10 ml), dried (magnesium sulphate), evaporated and purified by flash chromatography on a Biotage column eluting with 5-10% methanol in ethyl acetate. After trituration with ether the title compound was isolated as a white solid (61 mg).

LC/MS: Rt=2.60 min, [M+H]⁺ 452.1, 454.2

The following compounds were prepared in a similar manner by reaction of the appropriate acid with oxalyl chloride then treatment with an amine and pyridine.

| | | | LC/MS |
|---|---|---|---|
| Example No. | Structure | Name | data |
| 95 (E95) | 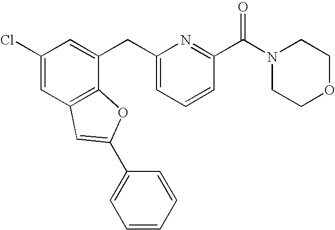 | 4-({6-[(5-Chloro-2- phenyl-1- benzofuran-7- yl)methyl]-2- pyridinyl}carbonyl) morpholine | Rt = 3.46 min, [M + H]⁺ 433.1, 435.1 |
| 96 (E96) | 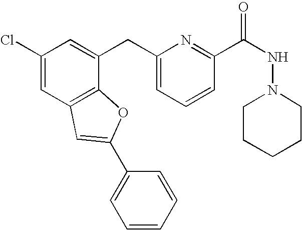 | 6-[(5-Chloro-2- phenyl-1- benzofuran-7- yl)methyl]-N-1- piperidinyl-2- pyridinecarboxamide | Rt = 3.87 min, [M + H]⁺ 446.2, 448.2 |
| 97 (E97) | 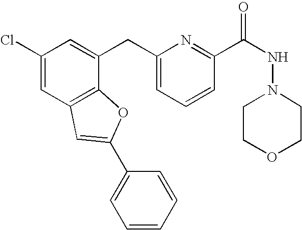 | 6-[(5-Chloro-2- phenyl-1- benzofuran-7- yl)methyl]-N-4- morpholinyl-2- pyridinecarboxamide | Rt = 3.45, [M + H]⁺ 448.1, 450.1 |
| 98 (E98) | 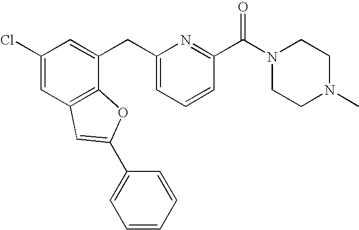 | 1-({6-[(5-Chloro-2- phenyl-1- benzofuran-7- yl)methyl]-2- pyridinyl}carbonyl)- 4-methylpiperazine | Rt = 2.29, [M + H]⁺ 446.2, 448.2 |
| 99 (E99) | 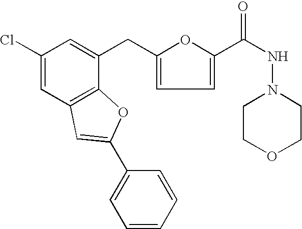 | 5-[(5-Chloro-2- phenyl-1- benzofuran-7- yl)methyl]-N-4- morpholinyl-2- furancarboxamide | Rt = 3.28 min, [M + H]⁺ 437.1, 439.1 |
| 100 (E100) | 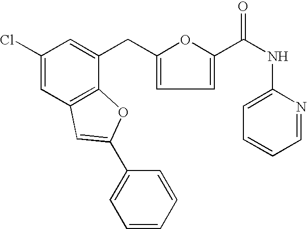 | 5-[(5-Chloro-2- phenyl-1- benzofuran-7- yl)methyl]-N-2- pyridinyl-2- furancarboxamide | Rt = 3.85 min, [M + H]⁺ 429.1, 431.1 |
| 101 (E101) | 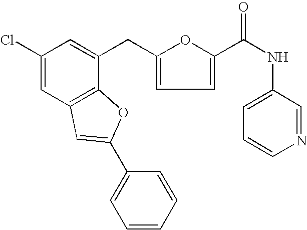 | 5-[(5-Chloro-2- phenyl-1- benzofuran-7- yl)methyl]-N-3- pyridinyl-2- furancarboxamide | Rt = 3.25 min, [M + H]⁺ 429.1, 431.1 |
| 102 (E102) | 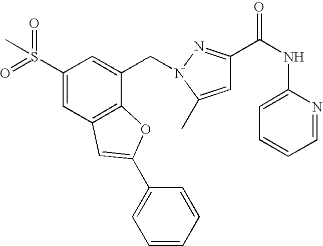 | 5-Methyl-1-{[5- (methylsulfonyl)-2- phenyl-1- benzofuran-7- yl]methyl}-N-2- pyridinyl-1H- pyrazole-3- carboxamide | Rt = 3.12 min, [M + H]⁺ 487.2 |
| 103 (E103) | 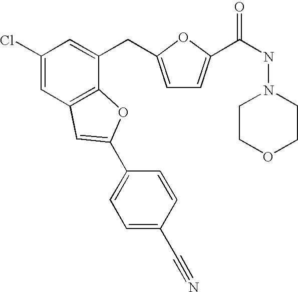 | 5-{[5-Chloro-2-(4- cyanophenyl)-1- benzofuran-7- yl]methyl}-N-4- morpholinyl-2- furancarboxamide | Rt = 3.18 min [M + H]⁺ 462.1 |

### Examples 104 & 105 (E104 & E105)

### 5-Methyl-1-{[5-(methylsulfonyl)-2-phenyl-1-benzofuran-7-yl]methyl}-N-4-morpholinyl-1H-pyrazole-3-carboxamide and 4-[(5-Methyl-1-{[5-(methylsulfonyl)-2-phenyl-1-benzofuran-7-yl]methyl}-1H-pyrazol-3-yl)carbonyl]morpholine

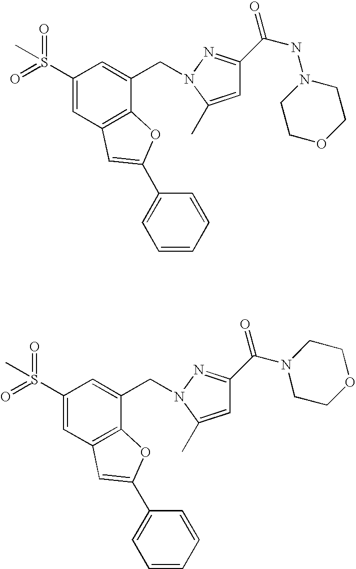

Oxalyl chloride (0.5 ml) was added to a stirred suspension of 5-methyl-1-{[5-(methylsulfonyl)-2-phenyl-1-benzofuran-7-yl]methyl}-1H-pyrazole-3-carboxylic acid (246 mg, 0.6 mmol) and DMF (1 drop) in dichloromethane (5 ml) and stirred at room temperature for 30 minutes. The solution was evaporated and azeotroped with toluene (10 ml). The residue was dissolved in dichloromethane (6 ml) and 3 ml of this solution was added to a solution of 4-aminomorpholine (51 mg, 0.5 mmol) and pyridine (0.1 ml) in dichloromethane (2 ml). After one hour the resulting solution was diluted with ethyl acetate (40 ml) washed with water (20 ml), dried (magnesium sulphate), evaporated, azeotroped with toluene and purified by flash chromatography on a Biotage column eluting with ethyl acetate to remove the faster running product changing to methanol/ethyl acetate (1:19) to elute the slower running product.

The faster running product was 4-[(5-methyl-1-{[5-(methylsulfonyl)-2-phenyl-1-benzofuran-7-yl]methyl}-1H-pyrazol-3-yl)carbonyl]morpholine (E105) (38 mg).

LC/MS: Rt=2.76 min, [M+H]⁺ 480.2

The slower running product was 5-methyl-1-{[5-(methylsulfonyl)-2-phenyl-1-benzofuran-7-yl]methyl}-N-4-morpholinyl-1H-pyrazole-3-carboxamide (E104) (80 mg).

LC/MS: Rt=2.58 min, [M+H]⁺ 495.2

### Example 106 & 107 (E106 & E107)

### 5-[(5-Chloro-2-phenyl-1-benzofuran-7-yl)methyl]-N-1-piperidinyl-2-furancarboxamide (E106) and 1-({5-[(5-Chloro-2-phenyl-1-benzofuran-7-yl)methyl]-2-furanyl}carbonyl)piperidine (E107)

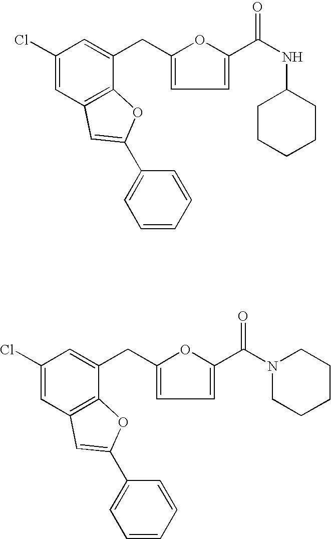

A mixture of 5-[(5-chloro-2-phenyl-1-benzofuran-7-yl)methyl]-2-furancarboxylic acid (71 mg, 0.2 mmol), 1-aminopiperidine (30 mg, 0.3 mmol), hydroxybenzotriazole (34 mg, 0.22 mmol) and EDAC (57 mg, 0.3 mmol) in DMF (2 ml) was stirred at room temperature for 4 hours. The resulting solution was diluted with ethyl acetate (30 ml) and water (40 ml) and the organic phase washed with 1M sodium hydroxide (15 ml) and water (3×20 ml) then dried (magnesium sulphate) and evaporated. The residue was purified by flash chromatography on a Biotage column eluting with 1:1 ethyl acetate/hexane to separate the two major products.

The faster running product was 1-({5-[(5-chloro-2-phenyl-1-benzofuran-7-yl)methyl]-2-furanyl}carbonyl)piperidine (E107) (13 mg)

LC/MS: Rt=3.95 min, [M+H]⁺ 420.2, 422.1

The slower running product was 5-[(5-chloro-2-phenyl-1-benzofuran-7-yl)methyl]-N-1-piperidinyl-2-furancarboxamide (E106) (31 mg).

LC/MS: Rt=3.63 min, [M+H]⁺ 435.1, 437.1

### Example 108 (E108)

### 1-({5-[(5-Chloro-2-phenyl-1-benzofuran-7-yl)methyl]-2-furanyl}carbonyl)-4-methylpiperazine hydrochloride

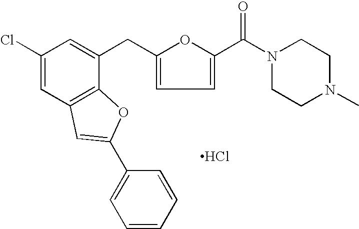

A mixture of 5-[(5-chloro-2-phenyl-1-benzofuran-7-yl)methyl]-2-furancarboxylic acid (71 mg, 0.2 mmol), N-methylpiperazine (30 mg, 0.3 mmol), hydroxybenzotriazole (34 mg, 0.22 mmol) and EDAC (57 mg, 0.3 mmol) in DMF (2 ml) was stirred at room temperature for 4 hours.

The resulting solution was diluted with ethyl acetate (30 ml) and water (40 ml) and the organic phase washed with 1M sodium hydroxide (15 ml) and water (3×20 ml) then dried (magnesium sulphate) and evaporated. The residue was dissolved in dichloromethane (5 ml), treated with 1M hydrogen chloride in ether, evaporated and triturated with ether to give the title compound as a pale yellow solid (69 mg).

Rt=2.37 min, [M+H]⁺ 435.1, 437.1

### Example 109 (E109)

### 4-({5-[(5-Chloro-2-phenyl-1-benzofuran-7-yl)methyl]-2-furanyl}carbonyl)morpholine

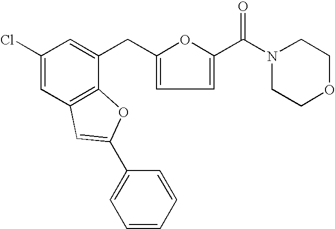

A mixture of 5-[(5-chloro-2-phenyl-1-benzofuran-7-yl)methyl]-2-furancarboxylic acid (71 mg, 0.2 mmol), morpholine (26 mg, 0.3 mmol), hydroxybenzotriazole (34 mg, 0.22 mmol) and EDAC (57 mg, 0.3 mmol) in DMF (2 ml) was stirred at room temperature for 4 hours. The resulting solution was diluted with ethyl acetate (30 ml) and water (30 ml) and the organic phase washed with saturated sodium bicarbonate (20 ml) and water (3×15 ml) then dried (magnesium sulphate) and evaporated. The residue was triturated with 1:1 ether/hexane to give the title compound as a white solid (61 mg).

Rt=3.56 min, [M+H]⁺ 422.1, 424.1

### Examples 110 & 111 (E110 & E111)

### 1-{[5-Chloro-2-(2,4-difluorophenyl)-1-benzofuran-7-yl]methyl}-5-methyl-N-4-morpholinyl-1H-pyrazole-3-carboxamide (E110) and 4-[(1-{[5-Chloro-2-(2,4-difluorophenyl)-1-benzofuran-7-yl]methyl}-5-methyl-1H-pyrazol-3-yl)carbonyl]morpholine (E111)

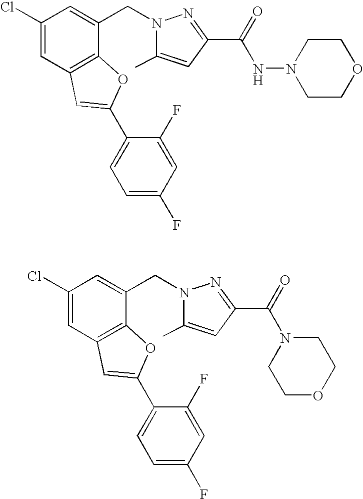

A suspension of 1-{[5-chloro-2-(2,4-difluorophenyl)-1-benzofuran-7-yl]methyl}-5-methyl-1H-pyrazole-3-carboxylic acid in dry dichloromethane (4 ml) and thionyl chloride (0.36 ml) was heated at 50° C. under argon for one hour to give a solution. The solution was evaporated to dryness and the residue dissolved in dichloromethane (2 ml). Triethylamine (0.165 ml) and 4-aminomorpholine were added and after ten minutes, the reaction mixture was diluted with dichloromethane and washed with 1:1 saturated sodium bicarbonate: water. The organic layer was then dried (MgSO₄) and evaporated to a pale brown solid. A slurry of the residue in dichloromethane was applied to a Biotage Si 25+M column and eluted with 60% ethyl acetate/hexane (500 ml) followed by 80% ethyl acetate/hexane (500 ml).

A compound (11 mg) was isolated and was shown to be 4-[(1-{[5-chloro-2-(2,4-difluorophenyl)-1-benzofuran-7-yl]methyl}-5-methyl-1H-pyrazol-3-yl)carbonyl]morpholine (E111)

LC/MS Rt=3.54 min. Molecular ion observed [M+H]⁺ 472, consistent with molecular formula C₂₄H₂₀N₃O₃³⁵ ClF₂

The column was further eluted with 2% methanol/ethyl acetate (500 ml) to afford 1-{[5-chloro-2-(2,4-difluorophenyl)-1-benzofuran-7-yl]methyl}-5-methyl-N-4-morpholinyl-1H-pyrazole-3-carboxamide (E110) as a white solid (96 mg).

LC/MS Rt=3.28 min. Molecular ion observed [M+H]⁺ 487, consistent with molecular formula C₂₄H₂₁N₄O₃³⁵ ClF₂

### Example 112 (E112)

### 1-{[5-chloro-2-(2,4-difluorophenyl)-1-benzofuran-7-yl]methyl}-N-(1,1-dimethylethyl)-5-methyl-1H-pyrazole-3-carboxamide

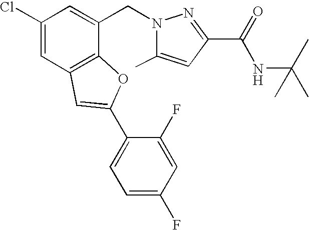

A suspension of 1-{[5-chloro-2-(2,4-difluorophenyl)-1-benzofuran-7-yl]methyl}-5-methyl-1H-pyrazole-3-carboxylic acid in dry dichloromethane (4 ml) and thionyl chloride (0.180 ml) was heated at 50° C. under argon for one hour to give a solution. The solution was evaporated to dryness and the residue dissolved in dichloromethane (2 ml) and cooled to 0° C. Triethylamine (0.083 ml) and ^{t}butylamine (0.031 ml) were added and after ten minutes, the reaction mixture was diluted with dichloromethane and washed with 1:1 saturated sodium bicarbonate: water and water. The organic layer was then dried (MgSO₄) and evaporated to a pale brown foam. The foam was dissolved in dichloromethane and applied to a Biotage Si 25+S column (pre-wetted with hexane) and eluted with 20% ethyl acetate/hexane (500 ml) taking 5 ml fractions. Fractions 24-44 were evaporated and dried to afford the title compound as a white solid (100 mg).

LC/MS Rt=3.78 min. Molecular ion observed [M+H]⁺ 458, consistent with molecular formula C₂₄H₂₂N₃O₂³⁵ ClF₂

### Example 113 (E113)

### 1-{[5-Chloro-2-(4-chlorophenyl)-1-benzofuran-7-yl]methyl}-5-methyl-N-4-morpholinyl-1H-pyrazole-3-carboxamide

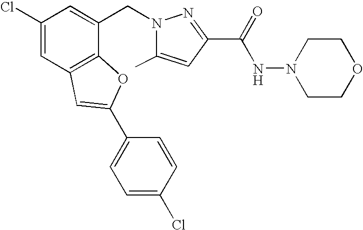

To a solution of 1-{[5-chloro-2-(4-chlorophenyl)-1-benzofuran-7-yl]methyl}-5-methyl-1H-pyrazole-3-carboxylic acid (50 mg) in N,N-dimethylformamide (2 ml) was added N-ethylmorpholine (0.064 ml), 4-aminomorpholine (0.014 ml), 1-hydroxybenzotriazole hydrate (26 mg) and N-(3-dimethylaminopropyl)-N′-ethylcarbodiimide (29 mg) and the reaction mixture was stirred overnight. The reaction mixture was diluted with ethyl acetate (30 ml) and washed with saturated sodium bicarbonate (20 ml) and water (3×15 ml) then dried (MgSO₄) and evaporated. The solid was dissolved in dichloromethane and applied to a Biotage Si 12+M column and purified using the Biotage SP4 (gradient method) to afford the title compound as an off-white solid (37 mg).

LC/MS Rt=3.31 min. Molecular ion observed [M+H]⁺ 485, consistent with molecular formula C₂₄H₂₂N₄O₃³⁵Cl₂

The following compounds were prepared in a similar manner using the appropriate carboxylic acid and amine;

| Example No. | Structure | Name | Data |
|---|---|---|---|
| 114 (E114) | 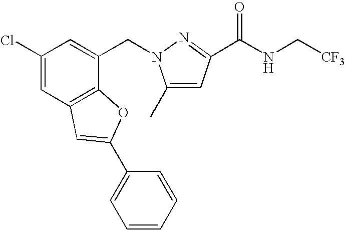 | 1-[(5-Chloro-2- phenyl-1- benzofuran-7- yl)methyl]-5- methyl-N-(2,2,2- trifluoroethyl)-1H- pyrazole-3- carboxamide | Rt = 3.52 min, [M + H]⁺ 448 |
| 115 (E115) | 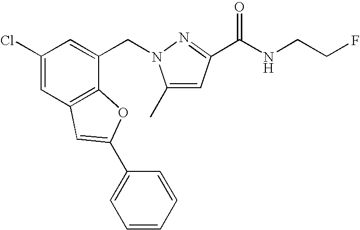 | 1[(5-Chloro-2- phenyl-1- benzofuran-7- yl)methyl]-N-(2- fluoroethyl)-5- methyl-1H- pyrazole-3- carboxamide | Rt = 3.33 min, [M + H]⁺ 412 |
| 116 (E116) | 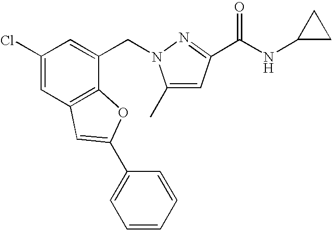 | 1-[(5-Chloro-2- phenyl-1- benzofuran-7- yl)methyl]-N- cyclopropyl-5- methyl-1H- pyrazole-3- carboxamide | Rt = 3.49 min [M + H]⁺ 406 |
| 117 (E117) | 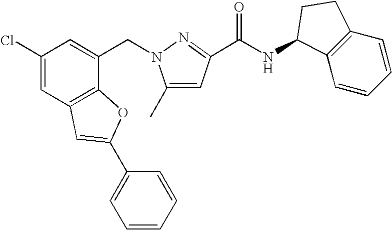 | 1-[(5-Chloro-2- phenyl-1- benzofuran-7- yl)methyl]-N-[(1S)- 2,3-dihydro-1H- inden-1-yl]-5- methyl-1H- pyrazole-3- carboxamide | Rt = 3.93 min [M + H]⁺ 482 |
| 118 (E118) | 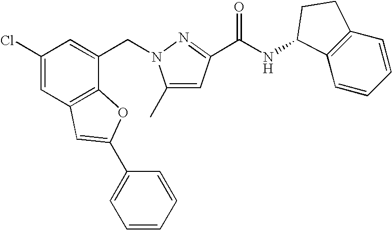 | 1-[(5-chloro-2- phenyl-1- benzofuran-7- yl)methyl]-N-[(1R)- 2,3-dihydro-1H- inden-1-yl]-5- methyl-1H- pyrazole-3- carboxamide | Rt = 3.93 min [M + H]⁺ 482 |
| 119 (E119) | 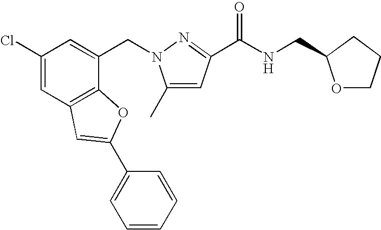 | 1-[(5-chloro-2- phenyl-1- benzofuran-7- yl)methyl]-5- methyl-N-[(2R)- tetrahydro-2- furanylmethyl]-1H- pyrazole-3- carboxamide | Rt = 3.53 min [M + H]⁺ 450 |

### Example 120 (E120)

### 1-{[5-Chloro-2-(4-chlorophenyl)-1-benzofuran-7-yl]methyl}-N-(1,1-dimethylethyl)-5-methyl-1H-pyrazole-3-carboxamide

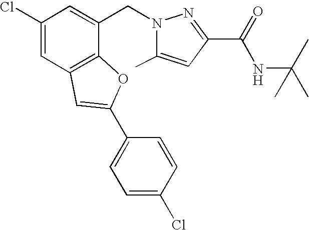

A suspension of 1-{[5-chloro-2-(4-chlorophenyl)-1-benzofuran-7-yl]methyl}-5-methyl-1H-pyrazole-3-carboxylic acid (100 mg) in dry dichloromethane (4 ml) and thionyl chloride (0.182 ml) was heated at 50° C. under argon for one hour to give a solution. The solution was evaporated to dryness and the residue dissolved in dichloromethane (2 ml) and cooled to 0° C. Triethylamine (0.084 ml) and ^{t}butylamine (0.032 ml) were added and after five minutes, the reaction mixture was diluted with dichloromethane (30 ml) and washed with 1:1 saturated sodium bicarbonate: water (2×20 ml) and water 20 ml). The organic layer was then dried (MgSO₄) and evaporated to an off-white solid. The solid was dissolved in dichloromethane and applied to a Biotage Si 12+M column and purified using the Biotage SP4 (gradient method) to afford the title compound as a cream coloured solid (86 mg).

LC/MS Rt=3.83 min. Molecular ion observed [M+H]⁺ 456, consistent with molecular formula C₂₄H₂₃N₃O₂³⁵Cl₂

### Example 121 (E121)

### 1-{[5-Chloro-2-(4-cyanophenyl)-1-benzofuran-7-yl]methyl}-5-methyl-N-4-morpholinyl-1H-pyrazole-3-carboxamide

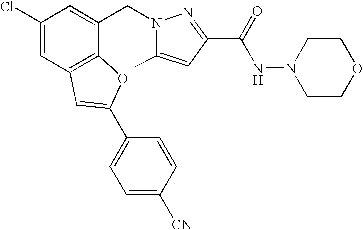

To a solution of 1-{[5-chloro-2-(4-cyanophenyl)-1-benzofuran-7-yl]methyl}-5-methyl-1H-pyrazole-3-carboxylic acid (150 mg) in N,N-dimethylformamide (6 ml) was added N-ethylmorpholine (0.193 ml), 4-aminomorpholine (0.044 ml), 1-hydroxybenzotriazole hydrate (80 mg) and N-(3-dimethylaminopropyl)-N′-ethylcarbodiimide (87 mg) and the reaction mixture was stirred overnight. The reaction mixture was diluted with ethyl acetate (90 ml) and washed with saturated sodium bicarbonate (100 ml) which was back extracted with ethyl acetate (50 ml). The combined organic layer was washed with water (3×30 ml). The product had precipitated out in the saturated sodium bicarbonate layer and was filtered off, washed with water and dried at 50° C. under vacuum to afford the title compound as a white solid (65 mg).

LC/MS Rt=2.99 min. Molecular ion observed [M+H]⁺ 476, consistent with molecular formula C₂₅H₂₂N₅O₃³⁵Cl

### Example 122 (E122)

### 1-{[5-Chloro-2-(4-cyanophenyl)-1-benzofuran-7-yl]methyl}-N-(1,1-dimethylethyl)-5-methyl-1H-pyrazole-3-carboxamide

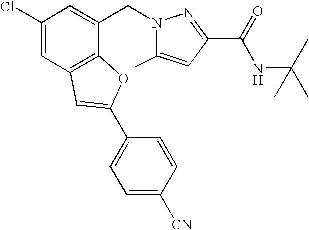

A suspension of 1-{[5-chloro-2-(4-cyanophenyl)-1-benzofuran-7-yl]methyl}-5-methyl-1H-pyrazole-3-carboxylic acid (150 mg) in dry dichloromethane (6 ml) and thionyl chloride (0.277 ml) was heated at 50° C. under argon for one hour to give a solution. The solution was evaporated to dryness and the residue dissolved in dichloromethane (3 ml) and cooled to 0° C. Triethylamine (0.127 ml) and ^{t}butylamine (0.048 ml) were added and after thirty minutes, the reaction mixture was diluted with dichloromethane (45 ml) and washed with 1:1 saturated sodium bicarbonate: water (2×30 ml) and water (30 ml). The organic layer was then dried (MgSO₄) and evaporated to a brown foam. The foam was dissolved in dichloromethane and applied to a Biotage Si 12+M column and purified using the Biotage SP4 (gradient method) and dried at 50° C. under vacuum to afford the title compound as a yellow coloured solid (30 mg).

LC/MS Rt=3.49 min. Molecular ion observed [M+H]⁺ 447, consistent with molecular formula C₂₅H₂₃N₄O₂³⁵Cl

### Example 123 (E123)

### 1-[(5-Chloro-2-phenyl-1-benzofuran-7-yl)methyl]-N-cyclobutyl-5-methyl-1H-pyrazole-3-carboxamide

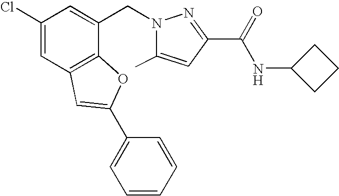

To a suspension of 1-[(5-chloro-2-phenyl-1-benzofuran-7-yl)methyl]-5-methyl-1H-pyrazole-3-carboxylic acid (150 mg) in N,N-dimethylformamide (6 ml) was added N-ethylmorpholine (0.208 ml), cyclobutylamine (0.09 ml), 1-hydroxybenzotriazole hydrate (86 mg) and N-(3-dimethylaminopropyl)-N′-ethylcarbodiimide (93 mg) and the reaction mixture was stirred overnight. Cyclobutylamine (0.09 ml) was added and the reaction mixture was stirred over three nights. The reaction mixture was diluted with ethyl acetate (40 ml) and washed with saturated sodium bicarbonate (25 ml) and water (2×25 ml) then dried (MgSO₄) and evaporated. The solid was stirred in diethyl ether for one hour then filtered off, washed with diethyl ether and dried at 45° C. under vacuum to afford the title compound (17 mg).

LC/MS Rt=3.67 min. Molecular ion observed [M+H]⁺ 420, consistent with molecular formula C₂₄H₂₂N₃O₂³⁵Cl

### Example 124 (E124)

### 1-[(5-Chloro-2-phenyl-1-benzofuran-7-yl)methyl]-N-cyclobutyl-5-methyl-1H-pyrazole-3-carboxamide

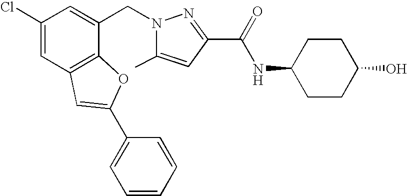

Prepared in a similar manner to 1-[(5-chloro-2-phenyl-1-benzofuran-7-yl)methyl]-N-cyclobutyl-5-methyl-1H-pyrazole-3-carboxamide using trans-4-aminocyclohexanol hydrochloride.

LC/MS Rt=3.18 min. Molecular ion observed [M+H]⁺ 464, consistent with molecular formula C₂₆H₂₆N₃O₃³⁵Cl

### Example 125 (E125)

### 1-[(5-Chloro-2-phenyl-1-benzofuran-7-yl)methyl]-N-(2,3-dihydro-1H-inden-2-yl)-5-methyl-1H-pyrazole-3-carboxamide

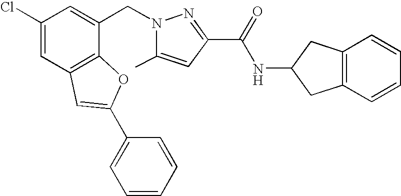

Prepared in a similar manner to 1-[(5-chloro-2-phenyl-1-benzofuran-7-yl)methyl]-N-cyclobutyl-5-methyl-1H-pyrazole-3-carboxamide using 2,3-dihydro-1H-inden-2-amine. Purified using the Biotage SP4 (t.l.c. method) to afford the title compound (115 mg).

LC/MS Rt=3.80 min. Molecular ion observed [M+H]⁺ 482, consistent with molecular formula C₂₉H₂₄N₃O₂³⁵Cl

### Example 126 (E126)

### 1-[(5-Chloro-2-phenyl-1-benzofuran-7-yl)methyl]-N-[(2R)-2-hydroxypropyl]-5-methyl-1H-pyrazole-3-carboxamide

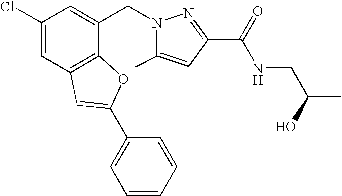

Prepared in a similar manner to 1-[(5-chloro-2-phenyl-1-benzofuran-7-yl)methyl]-N-cyclobutyl-5-methyl-1H-pyrazole-3-carboxamide using (R)-(−)-1-amino-2-propanol. Purified using the Biotage SP4 (gradient method) to afford the title compound (101 mg).

LC/MS Rt=3.12 min. Molecular ion observed [M+H]⁺ 424, consistent with molecular formula C₂₃H₂₂N₃O₃³⁵Cl

### Example 127 (E127)

### 1-[(5-Chloro-2-phenyl-1-benzofuran-7-yl)methyl]-N-[(2S)-2-hydroxypropyl]-5-methyl-1H-pyrazole-3-carboxamide

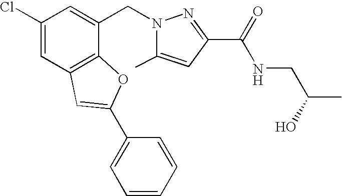

Prepared in a similar manner to 1-[(5-chloro-2-phenyl-1-benzofuran-7-yl)methyl]-N-cyclobutyl-5-methyl-1H-pyrazole-3-carboxamide using (S)-(+)-1-amino-2-propanol. Purified using the Biotage SP4 (gradient method) to afford the title compound (87 mg).

LC/MS Rt=3.12 min. Molecular ion observed [M+H]⁺ 424, consistent with molecular formula C₂₃H₂₂N₃O₃³⁵Cl

### Example 128 (E128)

### 1-[(5-Chloro-2-phenyl-1-benzofuran-7-yl)methyl]-5-methyl-N-(3,3,3-trifluoro-2-hydroxypropyl)-1H-pyrazole-3-carboxamide

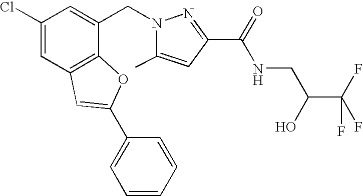

Prepared in a similar manner to 1-[(5-chloro-2-phenyl-1-benzofuran-7-yl)methyl]-N-cyclobutyl-5-methyl-1H-pyrazole-3-carboxamide using 3-amino-1,1,1′-trifluoro-2-propanol. Purified using the Biotage SP4 (gradient method) to afford the title compound (129 mg).

LC/MS Rt=3.42 min. Molecular ion observed [M+H]⁺ 478, consistent with molecular formula C₂₃H₁₉N₃O₃³⁵ClF₃

### Example 129 (E129)

### 5-Methyl-1-{[5-(methylsulfonyl)-2-phenyl-1-benzofuran-7-yl]methyl}-N-(tetrahydro-2H-pyran-4-yl)-1H-pyrazole-3-carboxamide

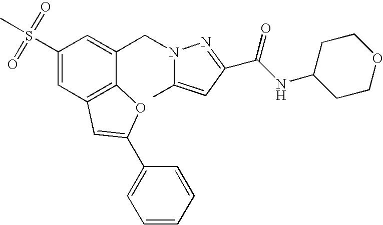

To a partial solution of 5-methyl-1-{[5-(methylsulfonyl)-2-phenyl-1-benzofuran-7-yl]methyl}-1H-pyrazole-3-carboxylic acid (83 mg) in N,N-dimethylformamide (1 ml) was added N-ethylmorpholine (0.101 ml), tetrahydro-2H-pyran-4-amine (40 mg), 1-hydroxybenzotriazole hydrate (42 mg) and N-(3-dimethylaminopropyl)-N′-ethylcarbodiimide (46 mg) and the reaction mixture was stirred over six nights. The reaction mixture was diluted with ethyl acetate (60 ml) and washed with saturated sodium bicarbonate (40 ml) and water (3×20 ml) then dried (MgSO₄) and evaporated to afford the title compound (17 mg) as a white foam.

LC/MS Rt=2.75 min. Molecular ion observed [M+H]⁺ 494, consistent with molecular formula C₂₆H₂₇N₃O₅S

### Example 130 (E130)

### N-(1,1-Dimethylethyl)-5-methyl-1-{[5-(methylsulfonyl)-2-phenyl-1-benzofuran-7-yl]methyl}-1H-pyrazole-3-carboxamide

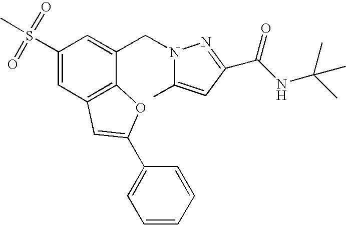

To a solution of 5-methyl-1-{[5-(methylsulfonyl)-2-phenyl-1-benzofuran-7-yl]methyl}-1H-pyrazole-3-carbonyl chloride (assumed 0.61 mmol) in dry dichloromethane (5 ml) was added triethylamine (0.203 ml) followed by ^{t}butylamine (73 mg) and the reaction mixture stirred at room temperature for thirty minutes. A 1:1 saturated sodium bicarbonate:water solution (2 ml) was added and the mixture stirred for thirty minutes. The reaction mixture was passed through a phase separator and the organic layer evaporated. The residue was purified by MDAP to afford the title compound (221 mg) as a white solid.

LC/MS Rt=3.17 min. Molecular ion observed [M+H]⁺ 466, consistent with molecular formula C₂₅H₂₇N₃O₄S

The examples in the following table were prepared as above and were purified either by MDAP or by Si column chromatography using the Biotage SP4 (t.l.c. method)

| Example No. | Structure | Name | Purification | LC/MS Data |
|---|---|---|---|---|
| 131 (E131) | 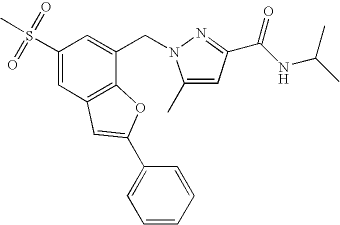 | 5-Methyl-N-(1- methylethyl)-1-{[5- (methylsulfonyl)-2- phenyl-1-benzofuran-7- yl]methyl}-1H-pyrazole- 3-carboxamide | Biotage SP4 | Rt = 2.94 min. [M + H]⁺ 452, C₂₄H₂₅N₃O₄S |
| 132 (E132) | 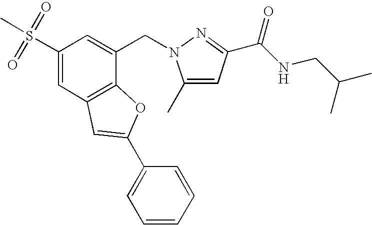 | 5-Methyl-N-(2- methylpropyl)-1-{[5- (methylsulfonyl)-2- phenyl-1-benzofuran-7- yl]methyl}-1H-pyrazole- 3-carboxamide | Biotage SP4 | Rt = 3.04 min. [M + H]⁺ 466, C₂₅H₂₇N₃O₄S |
| 133 (E133) | 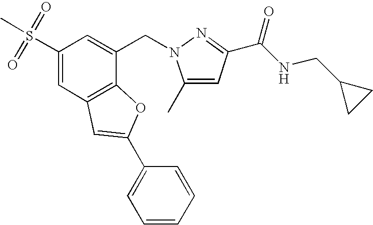 | N-(Cyclopropylmethyl)- 5-methyl-1-{[5- (methylsulfonyl)-2- phenyl-1-benzofuran-7- yl]methyl}-1H-pyrazole- 3-carboxamide | Biotage SP4 | Rt = 2.95 min. [M + H]⁺ 464, C₂₅H₂₅N₃O₄S |
| 134 (E134) | 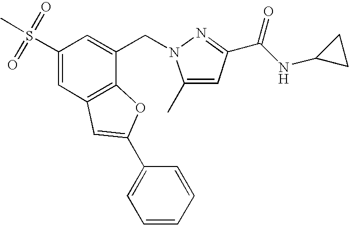 | N-Cyclopropyl-5- methyl-1-{[5- (methylsulfonyl)-2- phenyl-1-benzofuran-7- yl]methyl}-1H-pyrazole- 3-carboxamide | Biotage SP4 | Rt = 2.74 min. [MH+] 450, C₂₄H₂₃N₃O₄S |
| 135 (E135) | 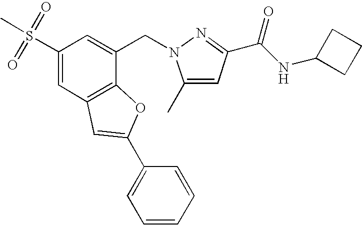 | N-Cyclobutyl-5-methyl- 1-{[5-(methylsulfonyl)-2- phenyl-1-benzofuran-7- yl]methyl}-1H-pyrazole- 3-carboxamide | Biotage SP4 | Rt = 2.95 min. [M + H]⁺ 464, C₂₅H₂₅N₃O₄S |
| 136 (E136) | 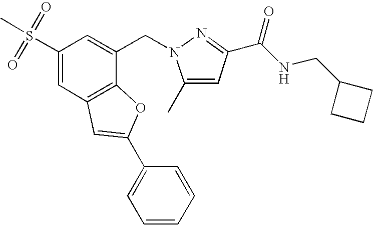 | N-(Cyclobutylmethyl)-5- methyl-1-{[5- (methylsulfonyl)-2- phenyl-1-benzofuran-7- yl]methyl}-1H-pyrazole- 3-carboxamide | Biotage SP4 | Rt = 3.15 min. [M + H]⁺ 478, C₂₆H₂₇N₃O₄S |
| 137 (E137) | 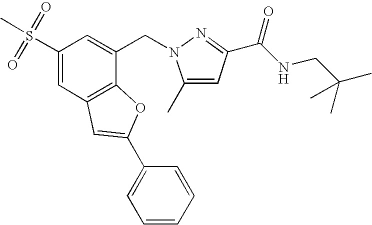 | N-(2,2-Dimethylpropyl)- 5-methyl-1-{[5- (methylsulfonyl)-2- phenyl-1-benzofuran-7- yl]methyl}-1H-pyrazole- 3-carboxamide | MDAP | Rt = 3.23 min. [M + H]⁺ 478, C₂₆H₂₇N₃O₄S |
| 138 (E138) | 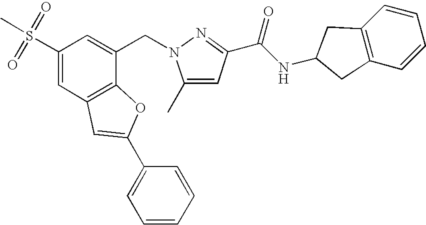 | N-(2,3-Dihydro-1H- inden-2-yl)-5-methyl-1- {[5-(methylsulfonyl)-2- phenyl-1-benzofuran-7- yl]methyl}-1H-pyrazole- 3-carboxamide | Biotage SP4 | Rt = 3.28 min. [M + H]⁺ 526, C₃₀H₂₇N₃O₄S |
| 139 (E139) | 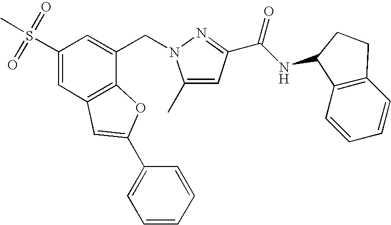 | N-[(1S)-2,3-Dihydro-1H- inden-1-yl]-5-methyl-1- {[5-(methylsulfonyl)-2- phenyl-1-benzofuran-7- yl]methyl}-1H-pyrazole- 3-carboxamide | Biotage SP4 | Rt = 3.32 min. [M + H]⁺ 526, C₃₀H₂₇N₃O₄S |
| 140 (E140) | 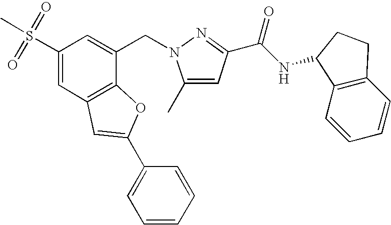 | N-[(1R)-2,3-Dihydro- 1H-inden-1-yl]-5- methyl-1-{[5- (methylsulfonyl)-2- phenyl-1-benzofuran-7- yl]methyl}-1H-pyrazole- 3-carboxamide | Biotage SP4 | Rt = 3.32 min. [M + H]⁺ 526, C₃₀H₂₇N₃O₄S |

### Example 141 (E141)

### N-{1-[(5-chloro-2-phenyl-1-benzofuran-7-yl)methyl]-5-methyl-1H-pyrazol-3-yl}-5-oxo-L-prolinamide

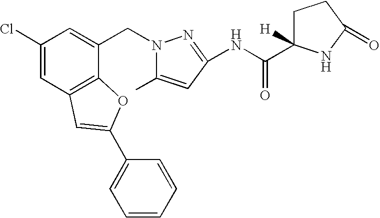

Solution of 1-[(5-chloro-2-phenyl-1-benzofuran-7-yl)methyl]-5-methyl-1H-pyrazol-3-amine (0.100 g, 0.3 mmol) in dry DCM (2.0 ml) was stirred at room temperature under an atmosphere of argon. EDAC (0.069 g, 0.36 mmol) and HOBt (0.048 g, 0.36 mmol) were added to the solution. L-pyroglutamic acid (0.046 g, 0.36 mmol) was added to the solution and stirring continued at room temperature for 18 hours. The solution was diluted with DCM, and washed with water. The solution was dried over Na₂SO₄, and the organics concentrated under reduced pressure. The residue was chromatographed [SiO₂, Hexane/EtOAc, 30-75%] to give the title compound (0.046 g).

LC/MS Rt=3.00 min [M+H]⁺ 449, 451

The following compounds were prepared in a similar manner, treating with trifluoroacetic acid where necessary to form the trifluoroacetate salt, or methansulfonic acid to form the methane sulfonate salt. In some cases, it was necessary to purify the compounds using MDAP.

| | | | LC/MS |
|---|---|---|---|
| Example no. | Structure | Name | Data |
| 142 (E142) | 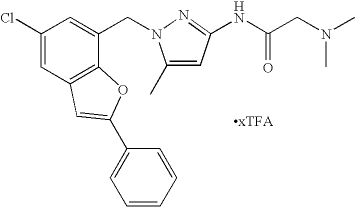 | N¹-{1-[(5-chloro-2- phenyl-1-benzofuran-7- yl)methyl]-5-methyl-1H- pyrazol-3-yl}-N²,N²- dimethylglycinamide | Rt = 2.50 min, [M + H]⁺ 423, 425 |
| 143 (E143) | 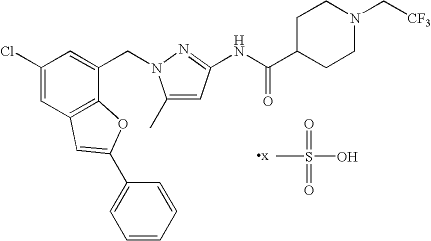 | N-{1-[(5-chloro-2-phenyl- 1-benzofuran-7- yl)methyl]-5-methyl-1H- pyrazol-3-yl}-1-(2,2,2- trifluoroethyl)-4- piperidinecarboxamide | Rt = 3.48 min [M + H]⁺ 531, 533 |
| 144 (E144) | 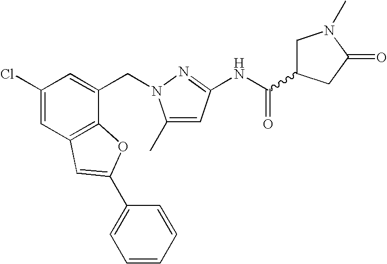 | N-{1-[(5-chloro-2-phenyl- 1-benzofuran-7- yl)methyl]-5-methyl-1H- pyrazol-3-yl}-1-methyl-5- oxo-3- pyrrolidinecarboxamide | Rt = 3.13 min, [M + H]⁺ 463, 465 |
| 145 (E145) | 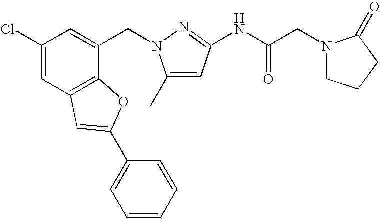 | N-{1-[(5-chloro-2-phenyl- 1-benzofuran-7- yl)methyl]-5-methyl-1H- pyrazol-3-yl}-2-(2-oxo-1- pyrrolidinyl)acetamide | Rt = 3.18 min |

### Example 146 (E146)

### (3R)—N-{1-[(5-chloro-2-phenyl-1-benzofuran-7-yl)methyl]-5-methyl-1H-pyrazol-3-yl}-1-(2,2,2-trifluoroethyl)-3-piperidinecarboxamide methane sulfonate

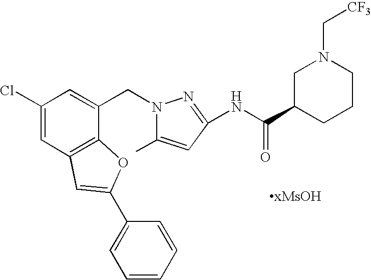

A solution of (3R)—N-{1-[(5-chloro-2-phenyl-1-benzofuran-7-yl)methyl]-5-methyl-1H-pyrazol-3-yl}-3-piperidinecarboxamide methane sulfonate (0.082 g, 0.13 mmol) in EtOH (0.5 ml) was stirred at room temperature under argon. 2,2,2-trifluoromethane sulfonate (0.014 g, 0.06 mmol) and NaHCO₃ (0.054 g, 0.65 mmol) were added and the mixture was stirred at room temperature for 1 hour. The reaction mixture was then heated to 60° C. and stirred for a further 18 hours (overnight). The mixture was allowed to cool to room temperature. The solution was diluted with EtOAc and organics washed with NaHCO₃ (sat. aq. soln.). Organics were dried over MgSO₄, filtered and concentrated under reduced pressure to give a yellow coloured oil. The residue was chromatographed [SiO₂; Hexane:EtOAc (50-100%)]. The residue obtained was treated with MsOH to salt.

LC/MS Rt=3.70 min [M+H]⁺ 531, 533

### Example 147 (E147)

### N-{1-[(5-chloro-2-phenyl-1-benzofuran-7-yl)methyl]-5-methyl-1H-pyrazol-3-yl}tetrahydro-2H-pyran-4-carboxamide

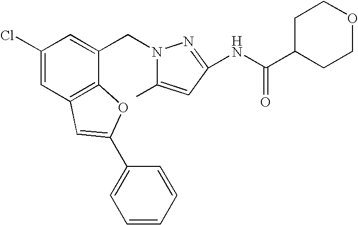

1-[(5-chloro-2-phenyl-1-benzofuran-7-yl)methyl]-5-methyl-1H-pyrazol-3-amine (150 mg, 0.444 mmol) was stirred in DMF (1.5 ml). EDAC 102 mg, 0.533 mmol), HOBt (74 mg, 0.533 mmol) and tetrahydropyran-4-yl-carboxylic acid (69 mg, 0.533 mmol) were added. The reaction mixture was stirred at room temperature under argon for 17 hours (overnight). The reaction mixture was then diluted with EtOAc and washed with NaHCO₃ (sat. soln.). The organics were washed with NaHCO₃ (sat. soln.). Organics were washed with further water (×3). Organic layer was dried over MgSO₄, filtered and concentrated to give a colourless oil. The oil was chromatographed [SiO₂, 50-100% EtOAc in Hexane] to give the title compound. (122 mg)

LC/MS Rt=3.29 min, [M+H]⁺ 450, 452

The following compound was prepared in a similar manner using the appropriate acid;

| | | | LC/MS |
|---|---|---|---|
| Example no. | Structure | Name | Data |
| 148 (E148) | 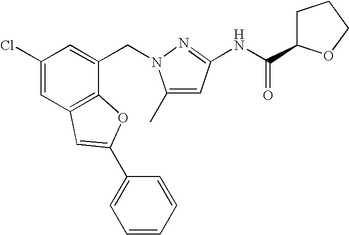 | (2R)-N-{1-[(5-chloro-2- phenyl-1-benzofuran-7- yl)methyl]-5-methyl-1H- pyrazol-3-yl}tetrahydro- 2-furancarboxamide | Rt = 3.34 min [M + H]⁺ 436, 438 |

### Example 149 (E149)

### N-{1-[(5-chloro-2-phenyl-1-benzofuran-7-yl)methyl]-5-methyl-1H-pyrazol-3-yl}-2-methylpropanamide

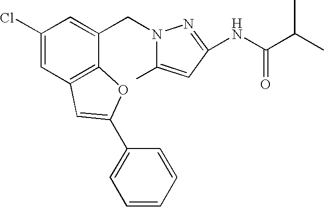

1-[(5-chloro-2-phenyl-1-benzofuran-7-yl)methyl]-5-methyl-1H-pyrazol-3-amine (150 mg, 0.444 mmol) was dissolved in dry DMF (1.8 ml). The slurry was stirred at room temperature under argon. Isobutyryl chloride (0.052 ml, 0.533 mmol) and Et₃N (0.149 ml, 1.07 mmol) were added and the solution stirred at room temperature under argon for 2 hours. The mixture was then diluted with EtOAc and washed with sat. soln. NaHCO₃. The organics were separated and the aqueous layer was washed with further EtOAc. The organics were combined and washed with water (×2). The organics were dried over MgSO₄, filtered and concentrated to give a colourless oil. The residue was chromatographed [SiO₂, 25-50% EtOAc/Hexane] to give the title compound. (55 mg)

LC/MS Rt=3.39 min, [M+H]⁺ 408, 410

### Example 150 (E150)

### N-(1-{[5-Chloro-2-(4-cyanophenyl)-1-benzofuran-7-yl]methyl}-5-methyl-1H-pyrazol-3-yl)-2-methylpropanamide

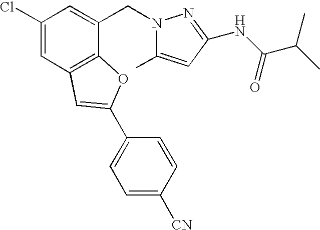

To a suspension of 4-{7-[(3-amino-5-methyl-1H-pyrazol-1-yl)methyl]-5-chloro-1-benzofuran-2-yl}benzonitrile (100 mg) in N,N-dimethylformamide (4 ml) was added N-ethylmorpholine (0.140 ml), isobutyric acid (0.031 ml), (1-hydroxybenzotriazole hydrate (58 mg) and N-(3-dimethylaminopropyl)-N′-ethylcarbodiimide (63 mg) and the reaction mixture stirred overnight. The reaction mixture was diluted with ethyl acetate (60 ml) and washed with saturated sodium bicarbonate (40 ml) and water (3×30 ml) then dried (MgSO₄) and evaporated to afford a yellow oil. The oil was dissolved in dichloromethane and applied to a Biotage Si 12+S column and purified using the Biotage SP4 (gradient method) then dried at 50° C. under vacuum to afford the title compound as a white solid (38 mg).

LC/MS Rt=3.38 min. Molecular ion observed [M+H]⁺ 433, consistent with molecular formula C₂₄H₂₁N₄O₂³⁵Cl

### Example 151 (E151)

### (2R)—N-(1-{[5-chloro-2-(4-cyanophenyl)-1-benzofuran-7-yl]methyl}-5-methyl-1H-pyrazol-3-yl)tetrahydro-2-furancarboxamide

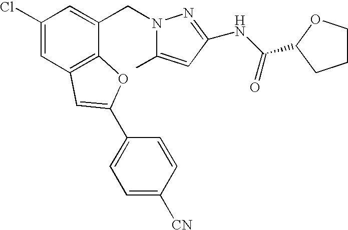

Prepared in a similar manner to N-(1-{[5-chloro-2-(4-cyanophenyl)-1-benzofuran-7-yl]methyl}-5-methyl-1H-pyrazol-3-yl)-2-methylpropanamide using (R)-(+)-tetrahydro-2-furoic acid.

LC/MS Rt=3.21 min. Molecular ion observed [M+H]⁺ 461, consistent with molecular formula C₂₅H₂₁N₄O₃³⁵Cl

### Example 152 (E152)

### N-(1-{[5-Chloro-2-(4-cyanophenyl)-1-benzofuran-7-yl]methyl}-5-methyl-1H-pyrazol-3-yl)tetrahydro-2H-pyran-4-carboxamide

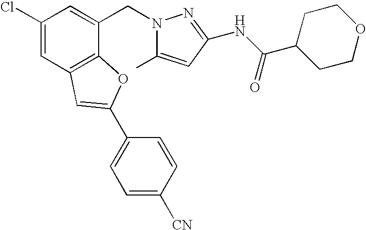

Prepared in a similar manner to N-(1-{[5-chloro-2-(4-cyanophenyl)-1-benzofuran-7-yl]methyl}-5-methyl-1H-pyrazol-3-yl)-2-methylpropanamide using tetrahydropyran-4-yl carboxylic acid.

LC/MS Rt=3.07 min. Molecular ion observed [M+H]⁺ 475, consistent with molecular formula C₂₆H₂₃N₄O₃³⁵Cl

### Example 153 (E153)

### N-{1-[(5-Cyano-2-phenyl-1-benzofuran-7-yl)methyl]-5-methyl-1H-pyrazol-3-yl}tetrahydro-2H-pyran-4-carboxamide

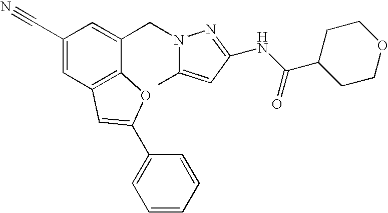

Prepared in a similar manner to N-(1-{[5-chloro-2-(4-cyanophenyl)-1-benzofuran-7-yl]methyl}-5-methyl-1H-pyrazol-3-yl)tetrahydro-2H-pyran-4-carboxamide using 7-[(3-amino-5-methyl-1H-pyrazol-1-yl)methyl]-2-phenyl-1-benzofuran-5-carbonitrile except that the title compound was further purified by trituration with diethyl ether.

LC/MS Rt=2.99 min. Molecular ion observed [M+H]⁺ 441, consistent with molecular formula C₂₆H₂₄N₄O₃

### Example 154 (E154)

### 2-Methylpropyl {1-[(5-chloro-2-phenyl-1-benzofuran-7-yl)methyl]-5-methyl-1H-pyrazol-3-yl}carbamate

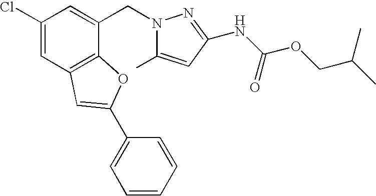

To a solution of 1-[(5-chloro-2-phenyl-1-benzofuran-7-yl)methyl]-5-methyl-1H-pyrazol-3-amine (100 mg) in dichloromethane (5 ml) and N-ethylmorpholine (0.075 ml) was added isobutyl chloroformate (0.05 ml) and the mixture stirred for three hours. The reaction mixture was diluted with dichloromethane and washed with water then dried (MgSO₄) and evaporated to a foam. The foam was dissolved in dichloromethane and applied to a Biotage Si 25+S column and purified using the Biotage SP4 (t.l.c. method) to afford the title compound (86 mg).

LC/MS t=3.85 min. Molecular ion observed [MH+] 438, consistent with molecular formula C₂₄H₂₄N₃O₃³⁵Cl

### Example 155 (E155)

### N-{1-[(5-Chloro-2-phenyl-1-benzofuran-7-yl)methyl]-5-methyl-1H-pyrazol-3-yl}-N′-(1,1-dimethylethyl)urea

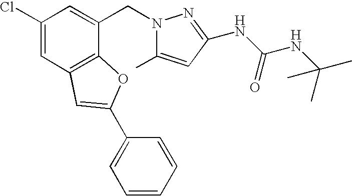

To a suspension of 1-[(5-chloro-2-phenyl-1-benzofuran-7-yl)methyl]-5-methyl-1H-pyrazol-3-amine (100 mg) in dichloromethane (5 ml) and tetrahydrofuran (1 ml) was added 1,1′-carbonyldiimidazole (57 mg) and the mixture stirred for one hour. The mixture was evaporated and the residue suspended in acetonitrile (5 ml) and ^{t}butylamine (26 mg) added. The reaction mixture was stirred for one hour then evaporated, mixed with water and extracted three times with dichloromethane. The combined organic layer was dried (MgSO₄) and evaporated to a white solid. The solid was purified by MDAP to afford the title compound as a white solid (26 mg).

LC/MS Rt=3.73 min. Molecular ion observed [M+H]⁺ 437, consistent with molecular formula C₂₄H₂₅N₄O₂³⁵Cl

### Example 156 (E156)

### 1,1-Dimethylethyl {5-[(5-chloro-2-phenyl-1-benzofuran-7-yl)methyl]-2-furanyl}carbamate

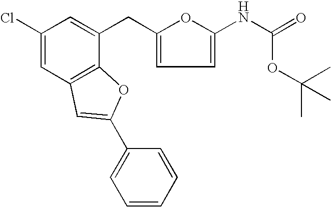

A mixture of 5-[(5-chloro-2-phenyl-1-benzofuran-7-yl)methyl]-2-furancarboxylic acid (88 mg, 0.25 mmol), triethylamine (28 mg, 0.28 mmol) and diphenylphosphoryl azide (76 mg, 0.275 mmol) in t-butanol (3 ml) was stirred and heated at 90° C. for 5 hours then evaporated to dryness. The residue was purified by flash chromatography on a Biotage column eluting with 1:14 ethyl acetate/hexane, then triturated with hexane to give the title compound as a yellow solid (37 mg).

LC/MS: Rt=4.05, [M+H]⁺ 424.1, 426.1

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

### Biological Activity at EP₁ and EP₃ Receptors

The ability of compounds to antagonize EP₁ & EP₃ receptors may be demonstrated using a functional calcium mobilization assay. Briefly, the antagonist properties of compounds are assessed by their ability to inhibit the mobilization of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilize. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analyzed with suitable curve-fitting software.

The human EP₁ or EP₃ calcium mobilization assay (hereafter referred to as ‘the calcium assay’) utilises Chinese hamster ovary-K1 (CHO—K1) cells into which a stable (PClN; BioTechniques 20 (1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analyzed by means of a computerised curve-fitting routine. The concentration of compound that elicits a half-maximal inhibition of the calcium mobilization induced by PGE₂ (pIC₅₀) may then be estimated.

### Binding Assay for the Human Prostanoid EP₁ Receptor

Competition assay using [³H]-PGE₂.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO—K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed (optional) three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analyzed using non linear curve fitting techniques to determine the concentration of compound producing 50% inhibition of specific binding (IC₅₀).

Alternatively a similar assay may be carried out using 3-{2-[5-Bromo-2-(2,4-difluoro-benzyloxy)-phenyl]-5-methyl-pyrrol-1-yl}-6-[³H₃-methoxy]methoxy-benzoic acid instead of [³H]-PGE₂.

For the binding assay using 3-{2-[5-Bromo-2-(2,4-difluoro-benzyloxy)-phenyl]-5-methyl-pyrrol-1-yl}-6-[³H₃-methoxy]methoxy-benzoic acid instead of [³H]-PGE₂ the assay is carried out using a similar procedure to that described above using [³H]-PGE₂ with the following changes:

The cell membranes, competing compounds and 3-{2-[5-Bromo-2-(2,4-difluoro-benzyloxy)-phenyl]-5-methyl-pyrrol-1-yl}-6-[³H₃-methoxy]methoxy-benzoic acid (0.2 nM final assay concentration) are incubated in a final volume of 400 μl for 45 min at 37° C. All reagents are prepared in assay buffer. Reactions are terminated by rapid vacuum filtration over GF/B filters using a Brandell cell harvester. The filters are washed with water at ambient temperature, dried and the radioactivity retained on the filters is measured by liquid scintillation counting in Packard TopCount scintillation counter.

The preparation of 3-{2-[5-Bromo-2-(2,4-difluoro-benzyloxy)-phenyl]-5-methyl-pyrrol-1-yl}-6-methoxy-benzoic acid is described in WO 03/101959 and in Hall et al, Biorg. Med. Chem. Lett., 2007, 17, 916-920. The tritiated version may be prepared via conventional routes, e.g. from 3-{2-[5-bromo-2-(2,4-difluoro-benzyloxy)-phenyl]-5-methyl-pyrrol-1-yl}-6-hydroxy-benzoic acid or 3-{2-[5-bromo-2-(2,4-difluoro-benzyloxy)-phenyl]-5-methyl-pyrrol-1-yl}-6-hydroxy-benzoic acid methyl ester.

### Biological Activity at TP Receptor

To determine if a compound has agonist or antagonist activity at the TP receptor a functional calcium mobilization assay may be performed. Briefly, the antagonist properties of compounds are assessed by their ability to inhibit the mobilization of intracellular calcium ([Ca²⁺]ᵢ) in response to activation of TP receptors by the stable TXA₂ mimetic U46619 (9,11-dideoxy-11α,9α-epoxy-methanoprostaglandin F2α; commercially available from e.g. Sigma-Aldrich). Increasing concentrations of antagonist reduce the amount of calcium that a given concentration of U46619 can mobilize. The net effect is to displace the U46619 concentration-effect curve. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analyzed with suitable curve-fitting software. The agonist activity of the compounds are determined by their ability to cause an increase in intracellular mobilization in the absence of U46619.

The human TP calcium mobilization assay utilises Chinese hamster ovary-K1 (CHO—K1) cells into which a stable (pCIN; BioTechniques 20 (1996): 102-110) vector containing TP cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 96-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of U46619 are then added to the plate in order to assess the antagonist properties of the compounds.

The data so generated may be analyzed by means of a computerised curve-fitting routine. The concentration of compound that elicits a half-maximal inhibition of the calcium mobilization induced by U46619 (pIC₅₀) may then be estimated, and the percentage activation caused by the compounds directly can be used to determine if there is any agonism present.

### Results

The compounds of Examples 1-117 and 119-156 were tested in the binding assay for the human prostanoid EP₁ receptor using 3-{2-[5-Bromo-2-(2,4-difluorobenzyloxy)-phenyl]-5-methyl-pyrrol-1-yl}-6-[³H₃-methoxy]methoxy-benzoic acid. The results are expressed as pIC₅₀ values. A pIC₅₀ is the negative logarithm₁₀ of the IC₅₀. The results given are averages of a number of experiments. The compounds of Examples 1-117 and 119-156 had a pIC₅₀ value ≧26.1.

The compounds of Examples 1-28 and 30-156 were tested in the human EP₁ calcium mobilization assay. The results are expressed as functional pKᵢ values. A functional pKᵢ is the negative logarithms of the antagonist dissociation constant as determined in the human EP₁ calcium mobilization assay. The results given are averages of a number of experiments. The compounds of Examples 37, 68, 112 and 156 did not show activity in this assay. All other compounds tested exhibited a functional pKᵢ value ≧2 5.8.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 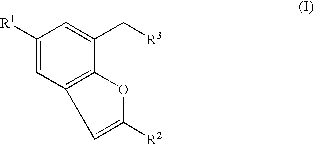
wherein:
R¹ is hydrogen, halogen, CN, CF₃ or SO₂CH₃;
R² is thienyl, thiazolyl, 1-methylimidazolyl, CH₂phenyl, phenyl optionally substituted by Cl, F or CN, or pyridyl optionally substituted by halogen;
R³ is 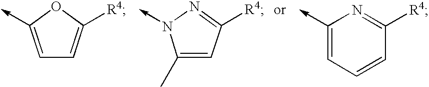
R⁴ is CO₂H, NHCO₂R⁵, CONR^{5a}R^{5b}, NHCOR⁷, NHCONR⁸R⁹, CONHSO₂R¹⁰, imidazole or tetrazole;
or R⁴ is an imidazole ring fused to give an optionally substituted bicyclic or tricyclic ring system;
R⁵ represents C₂₋₆ alkyl, or CH₂-heterocyclyl;
R^{6a} represents hydrogen; and
R^{6b} represents hydrogen; indane; NR¹¹R¹²; C₁₋₁₆alkyl optionally substituted by F, OH, OC₁₋₄alkyl or NR¹¹R¹²; phenyl optionally substituted by halogen, CH₂OH, CH₂NR¹¹R¹², or optionally substituted CH₂aliphatic heterocycle; optionally substituted (CH₂)ₘaliphatic heterocycle wherein m is 0, 1 or 2; or pyridine optionally substituted by CH₂aliphatic heterocycle or CONH-aliphatic heterocycle;
or R^{6a} and R^{6b} together with the nitrogen atom to which they are attached is an optionally substituted aliphatic heterocycle;
R⁷ is C₁₋₆alkyl; CH₂N(CH₃)₂; or optionally substituted (CH₂)ₙaliphatic heterocycle wherein n is 0, or 1;
R⁸ is hydrogen or C₁₋₄alkyl;
R⁹ is C₁₋₄alkyl;
R¹⁰ is C₁₋₄alkyl, aryl or heteroaryl;
R¹¹ is hydrogen or C₁₋₄alkyl; and
R¹² is hydrogen or C₁₋₄alkyl;
or derivatives thereof.

2. A compound according to claim 1 selected from the compounds of
1-{[5-Chloro-2-(2,4-dichlorophenyl)-1-benzofuran-7-yl]methyl}-5-methyl-1H-pyrazole-3-carboxylic acid,
1-{[5-chloro-2-(4-chloro-2-fluorophenyl)-1-benzofuran-7-yl]methyl}-5-methyl-1H-pyrazole-3-carboxylic acid,
1-{[5-chloro-2-(2-chlorophenyl)-1-benzofuran-7-yl]methyl}-5-methyl-1H-pyrazole-3-carboxylic acid,
1-{[5-chloro-2-(2-chloro-4-fluorophenyl)-1-benzofuran-7-yl]methyl}-5-methyl-1H-pyrazole-3-carboxylic acid,
1-{[5-chloro-2-(1,3-thiazol-2-yl)-1-benzofuran-7-yl]methyl}-5-methyl-1H-pyrazole-3-carboxylic acid,
1-[(5-Chloro-2-phenyl-1-benzofuran-7-yl)methyl]-5-methyl-1H-pyrazole-3-carboxylic acid,
6-[(5-Chloro-2-phenyl-1-benzofuran-7-yl)methyl]-2-pyridinecarboxylic acid,
5-[(5-Chloro-2-phenyl-1-benzofuran-7-yl)methyl]-2-furancarboxylic acid,
1-{[5-Chloro-2-(3-pyridinyl)-1-benzofuran-7-yl]methyl}-5-methyl-1H-pyrazole-3-carboxylic acid,
1-{[5-Chloro-2-(2-pyridinyl)-1-benzofuran-7-yl]methyl}-5-methyl-1H-pyrazole-3-carboxylic acid,
5-Methyl-1-{[5-(methylsulfonyl)-2-phenyl-1-benzofuran-7-yl]methyl}-1H-pyrazole-3-carboxylic acid,
1-{[5-Chloro-2-(3-thienyl)-1-benzofuran-7-yl]methyl}-5-methyl-1H-pyrazole-3-carboxylic acid,
1-{[5-Chloro-2-(1-methyl-1H-imidazol-5-yl)-1-benzofuran-7-yl]methyl}-5-methyl-1H-pyrazole-3-carboxylic acid,
1-{[5-Chloro-2-(Phenylmethyl)-1-benzofuran-7-yl]methyl}-5-methyl-1H-pyrazole-3-carboxylic acid,
1-{[5-Chloro-2-(2-cyanophenyl)-1-benzofuran-7-yl]methyl}-5-methyl-1H-pyrazole-3-carboxylic acid,
1-{[5-Chloro-2-(4-cyanophenyl)-1-benzofuran-7-yl]methyl}-5-methyl-1H-pyrazole-3-carboxylic acid,
1-[(5-Chloro-2-phenyl-1-benzofuran-7-yl)methyl]-5-methyl-1H-pyrazole-3-carboxylic acid,
1-{[5-chloro-2-(4-fluorophenyl)-1-benzofuran-7-yl]methyl}-5-methyl-1H-pyrazole-3-carboxylic acid,
1-[(5-Cyano-2-phenyl-1-benzofuran-7-yl)methyl]-5-methyl-1H-pyrazole-3-carboxylic acid,
1-{[5-Chloro-2-(2,4-difluorophenyl)-1-benzofuran-7-yl]methyl}-5-methyl-1H-pyrazole-3-carboxylic acid,
1-{[5-chloro-2-(5-fluoro-2-pyridinyl)-1-benzofuran-7-yl]methyl}-5-methyl-1H-pyrazole-3-carboxylic acid,
1-{[5-Chloro-2-(4-chlorophenyl)-1-benzofuran-7-yl]methyl}-5-methyl-1H-pyrazole-3-carboxylic acid,
1-[(5-Chloro-2-phenyl-1-benzofuran-7-yl)methyl]-5-methyl-N-[5-(1-piperazinylmethyl)-2-pyridinyl]-1H-pyrazole-3-carboxamide hydrochloride,
1-[(5-Chloro-2-phenyl-1-benzofuran-7-yl)methyl]-N-{4-[(ethylamino)methyl]phenyl}-5-methyl-1H-pyrazole-3-carboxamide hydrochloride,
1-[(5-Chloro-2-phenyl-1-benzofuran-7-yl)methyl]-N-{4-[(4-hydroxy-1-piperidinyl)methyl]phenyl}-5-methyl-1H-pyrazole-3-carboxamide,
1-[(5-Chloro-2-phenyl-1-benzofuran-7-yl)methyl]-5-methyl-N-[4-(1-pyrrolidinylmethyl)phenyl]-1H-pyrazole-3-carboxamide,
1-[(5-Chloro-2-phenyl-1-benzofuran-7-yl)methyl]-5-methyl-N-(4-{[(1-methylethyl)amino]methyl}phenyl)-1H-pyrazole-3-carboxamide,
1-[(5-Chloro-2-phenyl-1-benzofuran-7-yl)methyl]-5-methyl-N-[4-(1-piperazinyl)phenyl]-1H-pyrazole-3-carboxamide,
6-[(5-Chloro-2-phenyl-1-benzofuran-7-yl)methyl]-N-[4-(1-pyrrolidinylmethyl)phenyl]-2-pyridinecarboxamide,
6-[(5-Chloro-2-phenyl-1-benzofuran-7-yl)methyl]-N-[4-(4-morpholinylmethyl)phenyl]-2-pyridinecarboxamide,
1-{[5-chloro-2-(2,4-dichlorophenyl)-1-benzofuran-7-yl]methyl}-5-methyl-N-(4-piperidinylmethyl)-1H-pyrazole-3-carboxamide hydrochloride,
1-{[5-Chloro-2-(2-chlorophenyl)-1-benzofuran-7-yl]methyl}-5-methyl-N-(4-piperidinylmethyl)-1H-pyrazole-3-carboxamide hydrochloride,
(3R)—N-{1-[(5-chloro-2-phenyl-1-benzofuran-7-yl)methyl]-5-methyl-1H-pyrazol-3-yl}-3-piperidinecarboxamide methanesulfonate,
N-{1-[(5-Chloro-2-phenyl-1-benzofuran-7-yl)methyl]-5-methyl-1H-pyrazol-3-yl}-3-azetidinecarboxamide methanesulfonate,
N-{1-[(5-Chloro-2-phenyl-1-benzofuran-7-yl)methyl]-5-methyl-1H-pyrazol-3-yl}-2-[(2R)-2-pyrrolidinyl]acetamide methanesulfonate,
5-Methyl-1-{[5-(methylsulfonyl)-2-phenyl-1-benzofuran-7-yl]methyl}-N-(4-piperidinylmethyl)-1H-pyrazole-3-carboxamide hydrochloride,
6-[(5-Chloro-2-phenyl-1-benzofuran-7-yl)methyl]-N-(4-piperidinylmethyl)-2-pyridinecarboxamide hydrochloride,
5-{[5-Chloro-2-(4-cyanophenyl)-1-benzofuran-7-yl]methyl}-N-(4-piperidinylmethyl)-2-furancarboxamide hydrochloride,
1-[(5-Cyano-2-phenyl-1-benzofuran-7-yl)methyl]-5-methyl-N-(4-piperidinylmethyl)-1H-pyrazole-3-carboxamide hydrochloride,
5-[(5-Chloro-2-phenyl-1-benzofuran-7-yl)methyl]-N-(4-piperidinylmethyl)-2-furancarboxamide hydrochloride,
1-{[5-Chloro-2-(2,4-difluorophenyl)-1-benzofuran-7-yl]methyl}-5-methyl-N-[4-(1-piperazinylmethyl)phenyl]-1H-pyrazole-3-carboxamide hydrochloride,
1-{[5-Chloro-2-(2,4-difluorophenyl)-1-benzofuran-7-yl]methyl}-5-methyl-N-(4-piperidinylmethyl)-1H-pyrazole-3-carboxamide hydrochloride,
1-{[5-Chloro-2-(4-chlorophenyl)-1-benzofuran-7-yl]methyl}-5-methyl-N-(4-piperidinylmethyl)-1H-pyrazole-3-carboxamide hydrochloride,
1-{[(5-Chloro-2-(4-cyanophenyl)-1-benzofuran-7-yl]methyl}-5-methyl-N-(4-piperidinylmethyl)-1H-pyrazole-3-carboxamide hydrochloride,
6-[({1-[(5-Chloro-2-phenyl-1-benzofuran-7-yl)methyl]-5-methyl-1H-pyrazol-3-yl}carbonyl)amino]-N-(4-piperidinylmethyl)-3-pyridinecarboxamide hydrochloride,
1-[(5-Chloro-2-phenyl-1-benzofuran-7-yl)methyl]-5-methyl-N-[(3R)-3-pyrrolidinyl]-1H-pyrazole-3-carboxamide,
1-[(5-chloro-2-phenyl-1-benzofuran-7-yl)methyl]-5-methyl-N-(4-piperidinylmethyl)-1H-pyrazole-3-carboxamide hydrochloride,
1-[(5-Chloro-2-phenyl-1-benzofuran-7-yl)methyl]-5-methyl-N-4-piperidinyl-1H-pyrazole-3-carboxamide,
1-{[5-Chloro-2-(2-chloro-4-fluorophenyl)-1-benzofuran-7-yl]methyl}-5-methyl-N-(4-piperidinylmethyl)-1H-pyrazole-3-carboxamide hydrochloride,
(2S)—N-{(2Z)-3-[({5-Chloro-2-[(1E,3Z)-1-ethenyl-1,3-pentadien-1-yl]-1-benzofuran-7-yl}methyl)(methyl)amino]-1-methylidene-2-buten-1-yl}-2-piperidinecarboxamide hydrochloride,
N-{1-[(5-Chloro-2-phenyl-1-benzofuran-7-yl)methyl]-5-methyl-1H-pyrazol-3-yl}-4-piperidinecarboxamide hydrochloride,
N-{1-[(5-Chloro-2-phenyl-1-benzofuran-7-yl)methyl]-5-methyl-1H-pyrazol-3-yl}-L-prolinamide hydrochloride,
N-{1-[(5-Chloro-2-phenyl-1-benzofuran-7-yl)methyl]-5-methyl-1H-pyrazol-3-yl}-2-(4-piperidinyl)acetamide,
N-{1-[(5-Chloro-2-phenyl-1-benzofuran-7-yl)methyl]-5-methyl-1H-pyrazol-3-yl}-4-fluoro-4-piperidinecarboxamide hydrochloride,
1-Acetyl-N-{1-[(5-chloro-2-phenyl-1-benzofuran-7-yl)methyl]-5-methyl-1H-pyrazol-3-yl}-3-azetidinecarboxamide,
2-[(2S)-1-Acetyl-2-pyrrolidinyl]-N-{1-[(5-chloro-2-phenyl-1-benzofuran-7-yl)methyl]-5-methyl-1H-pyrazol-3-yl}acetamide,
N-[(3R)-1-Acetyl-3-pyrrolidinyl]-1-[(5-chloro-2-phenyl-1-benzofuran-7-yl)methyl]-5-methyl-1H-pyrazole-3-carboxamide,
1-[(5-chloro-2-phenyl-1-benzofuran-7-yl)methyl]-N-[(1-ethyl-4-piperidinyl)methyl]-5-methyl-1H-pyrazole-3-carboxamide,
N-{1-[(5-chloro-2-phenyl-1-benzofuran-7-yl)methyl]-5-methyl-1H-pyrazol-3-yl}-1-(1-methylethyl)-L-prolinamide hydrochloride,
1-{[5-Chloro-2-(2,4-dichlorophenyl)-1-benzofuran-7-yl]methyl}-5-methyl-1H-pyrazole-3-carboxamide,
1-[(5-chloro-2-phenyl-1-benzofuran-7-yl)methyl]-5-methyl-N-4-morpholinyl-1H-pyrazole-3-carboxamide,
1-{[5-chloro-2-(2-chlorophenyl)-1-benzofuran-7-yl]methyl}-5-methyl-N-4-morpholinyl-1H-pyrazole-3-carboxamide,
1-{[5-chloro-2-(4-chloro-2-fluorophenyl)-1-benzofuran-7-yl]methyl}-5-methyl-N-4-morpholinyl-1H-pyrazole-3-carboxamide,
1-[(5-cyano-2-phenyl-1-benzofuran-7-yl)methyl]-5-methyl-N-4-morpholinyl-1H-pyrazole-3-carboxamide,
1-[(5-Chloro-2-phenyl-1-benzofuran-7-yl)methyl]-N-(1,1-dimethylethyl)-5-methyl-1H-pyrazole-3-carboxamide,
1-[(5-chloro-2-phenyl-1-benzofuran-7-yl)methyl]-5-methyl-N-(tetrahydro-2H-pyran-4-yl)-1H-pyrazole-3-carboxamide,
1-[(5-chloro-2-phenyl-1-benzofuran-7-yl)methyl]-5-methyl-N-(tetrahydro-2H-pyran-4-ylmethyl)-1H-pyrazole-3-carboxamide,
1-[(5-chloro-2-phenyl-1-benzofuran-7-yl)methyl]-5-methyl-N-3-pyridinyl-1H-pyrazole-3-carboxamide,
1-[(5-chloro-2-phenyl-1-benzofuran-7-yl)methyl]-5-methyl-N-4-pyridinyl-1H-pyrazole-3-carboxamide,
1-[(5-chloro-2-phenyl-1-benzofuran-7-yl)methyl]-5-methyl-N-[2-(4-morpholinyl)ethyl]-1H-pyrazole-3-carboxamide,
1-[(5-chloro-2-phenyl-1-benzofuran-7-yl)methyl]-N-(2-hydroxyethyl)-5-methyl-1H-pyrazole-3-carboxamide,
1-[(5-chloro-2-phenyl-1-benzofuran-7-yl)methyl]-5-methyl-N-[2-(methyloxy)ethyl]-1H-pyrazole-3-carboxamide,
1-[(5-chloro-2-phenyl-1-benzofuran-7-yl)methyl]-N-(2-hydroxy-1,1-dimethylethyl)-5-methyl-1H-pyrazole-3-carboxamide,
1-[(5-chloro-2-phenyl-1-benzofuran-7-yl)methyl]-N-(cyclobutylmethyl)-5-methyl-1H-pyrazole-3-carboxamide,
1-[(5-chloro-2-phenyl-1-benzofuran-7-yl)methyl]-N-(2,2-dimethylpropyl)-5-methyl-1H-pyrazole-3-carboxamide,
1-[(5-chloro-2-phenyl-1-benzofuran-7-yl)methyl]-5-methyl-1H-pyrazole-3-carboxamide,
1-{[5-chloro-2-(2-chloro-4-fluorophenyl)-1-benzofuran-7-yl]methyl}-5-methyl-N-4-morpholinyl-1H-pyrazole-3-carboxamide,
1-[(5-Chloro-2-phenyl-1-benzofuran-7-yl)methyl]-5-methyl-N-(1-methylethyl)-1H-pyrazole-3-carboxamide,
1-[(5-chloro-2-phenyl-1-benzofuran-7-yl)methyl]-5-methyl-N-2-pyridinyl-1H-pyrazole-3-carboxamide,
1-[(5-Chloro-2-phenyl-1-benzofuran-7-yl)methyl]-5-methyl-N-1-piperidinyl-1H-pyrazole-3-carboxamide,
1-{[5-Chloro-2-(4-fluorophenyl)-1-benzofuran-7-yl]methyl}-5-methyl-N-4-morpholinyl-1H-pyrazole-3-carboxamide,
1-{[5-Chloro-2-(3-pyridinyl)-1-benzofuran-7-yl]methyl}-5-methyl-N-(1-methylethyl)-1H-pyrazole-3-carboxamide,
1-{[5-Chloro-2-(2-pyridinyl)-1-benzofuran-7-yl]methyl}-5-methyl-N-(1-methylethyl)-1H-pyrazole-3-carboxamide,
6-[(5-Chloro-2-phenyl-1-benzofuran-7-yl)methyl]-N-(1-methylethyl)-2-pyridinecarboxamide,
6-[(5-Chloro-2-phenyl-1-benzofuran-7-yl)methyl]-2-pyridinecarboxamide,
5-[(5-Chloro-2-phenyl-1-benzofuran-7-yl)methyl]-2-furancarboxamide,
5-[(5-Chloro-2-phenyl-1-benzofuran-7-yl)methyl]-N′,N′-dimethyl-2-furancarbohydrazide,
5-Methyl-1-{[5-(methylsulfonyl)-2-phenyl-1-benzofuran-7-yl]methyl}-1H-pyrazole-3-carboxamide,
5-[(5-Chloro-2-phenyl-1-benzofuran-7-yl)methyl]-N-[2-(dimethylamino)ethyl]-2-furancarboxamide hydrochloride,
5-{[5-Chloro-2-(phenylmethyl)-1-benzofuran-7-yl]methyl}-2-furancarboxamide,
1-{[5-Chloro-2-(3-thienyl)-1-benzofuran-7-yl]methyl}-5-methyl-1H-pyrazole-3-carboxamide,
1-{[5-Chloro-2-(1-methyl-1H-imidazol-5-yl)-1-benzofuran-7-yl]methyl}-5-methyl-1H-pyrazole-3-carboxamide,
5-{[5-Chloro-2-(4-cyanophenyl)-1-benzofuran-7-yl]methyl}-2-furancarboxamide,
1-[(5-Cyano-2-phenyl-1-benzofuran-7-yl)methyl]-5-methyl-1H-pyrazole-3-carboxamide,
1-{[5-Chloro-2-(2-pyridinyl)-1-benzofuran-7-yl]methyl}-5-methyl-N-4-morpholinyl-1H-pyrazole-3-carboxamide,
4-({6-[(5-Chloro-2-phenyl-1-benzofuran-7-yl)methyl]-2-pyridinyl}carbonyl)morpholine,
6-[(5-Chloro-2-phenyl-1-benzofuran-7-yl)methyl]-N-1-piperidinyl-2-pyridinecarboxamide,
6-[(5-Chloro-2-phenyl-1-benzofuran-7-yl)methyl]-N-4-morpholinyl-2-pyridinecarboxamide,
1-({6-[(5-Chloro-2-phenyl-1-benzofuran-7-yl)methyl]-2-pyridinyl}carbonyl)-4-methylpiperazine,
5-[(5-Chloro-2-phenyl-1-benzofuran-7-yl)methyl]-N-4-morpholinyl-2-furancarboxamide,
5-[(5-Chloro-2-phenyl-1-benzofuran-7-yl)methyl]-N-2-pyridinyl-2-furancarboxamide,
5-[(5-Chloro-2-phenyl-1-benzofuran-7-yl)methyl]-N-3-pyridinyl-2-furancarboxamide,
5-Methyl-1-{[5-(methylsulfonyl)-2-phenyl-1-benzofuran-7-yl]methyl}-N-2-pyridinyl-1H-pyrazole-3-carboxamide,
5-{[5-Chloro-2-(4-cyanophenyl)-1-benzofuran-7-yl]methyl}-N-4-morpholinyl-2-furancarboxamide,
5-Methyl-1-{[5-(methylsulfonyl)-2-phenyl-1-benzofuran-7-yl]methyl}-N-4-morpholinyl-1H-pyrazole-3-carboxamide,
4-[(5-Methyl-1-{[5-(methylsulfonyl)-2-phenyl-1-benzofuran-7-yl]methyl}-1H-pyrazol-3-yl)carbonyl]morpholine,
5-[(5-Chloro-2-phenyl-1-benzofuran-7-yl)methyl]-N-1-piperidinyl-2-furancarboxamide,
1-({5-[(5-Chloro-2-phenyl-1-benzofuran-7-yl)methyl]-2-furanyl}carbonyl)piperidine,
1-({5-[(5-Chloro-2-phenyl-1-benzofuran-7-yl)methyl]-2-furanyl}carbonyl)-4-methylpiperazine hydrochloride,
4-({5-[(5-Chloro-2-phenyl-1-benzofuran-7-yl)methyl]-2-furanyl}carbonyl)morpholine,
1-{[5-Chloro-2-(2,4-difluorophenyl)-1-benzofuran-7-yl]methyl}-5-methyl-N-4-morpholinyl-1H-pyrazole-3-carboxamide,
4-[(1-{[5-Chloro-2-(2,4-difluorophenyl)-1-benzofuran-7-yl]methyl}-5-methyl-1H-pyrazol-3-yl)carbonyl]morpholine,
1-{[5-Chloro-2-(2,4-difluorophenyl)-1-benzofuran-7-yl]methyl}-N-(1,1-dimethylethyl)-5-methyl-1H-pyrazole-3-carboxamide,
1-{[5-Chloro-2-(4-chlorophenyl)-1-benzofuran-7-yl]methyl}-5-methyl-N-4-morpholinyl-1H-pyrazole-3-carboxamide,
1-[(5-Chloro-2-phenyl-1-benzofuran-7-yl)methyl]-5-methyl-N-(2,2,2-trifluoroethyl)-1H-pyrazole-3-carboxamide,
1-[(5-Chloro-2-phenyl-1-benzofuran-7-yl)methyl]-N-(2-fluoroethyl)-5-methyl-1H-pyrazole-3-carboxamide,
1-[(5-Chloro-2-phenyl-1-benzofuran-7-yl)methyl]-N-cyclopropyl-5-methyl-1H-pyrazole-3-carboxamide,
1-[(5-Chloro-2-phenyl-1-benzofuran-7-yl)methyl]-N-[(1S)-2,3-dihydro-1H-inden-1-yl]-5-methyl-1H-pyrazole-3-carboxamide,
1-[(5-chloro-2-phenyl-1-benzofuran-7-yl)methyl]-N-[(1R)-2,3-dihydro-1H-inden-1-yl]-5-methyl-1H-pyrazole-3-carboxamide,
1-[(5-chloro-2-phenyl-1-benzofuran-7-yl)methyl]-5-methyl-N-[(2R)-tetrahydro-2-furanylmethyl]-1H-pyrazole-3-carboxamide,
1-{[5-Chloro-2-(4-chlorophenyl)-1-benzofuran-7-yl]methyl}-N-(1,1-dimethylethyl)-5-methyl-1H-pyrazole-3-carboxamide,
1-{[5-Chloro-2-(4-cyanophenyl)-1-benzofuran-7-yl]methyl}-5-methyl-N-4-morpholinyl-1H-pyrazole-3-carboxamide,
1-{[5-Chloro-2-(4-cyanophenyl)-1-benzofuran-7-yl]methyl}-N-(1,1-dimethylethyl)-5-methyl-1H-pyrazole-3-carboxamide,
1-[(5-Chloro-2-phenyl-1-benzofuran-7-yl)methyl]-N-cyclobutyl-5-methyl-1H-pyrazole-3-carboxamide,
1-[(5-Chloro-2-phenyl-1-benzofuran-7-yl)methyl]-N-cyclobutyl-5-methyl-1H-pyrazole-3-carboxamide,
1-[(5-Chloro-2-phenyl-1-benzofuran-7-yl)methyl]-N-(2,3-dihydro-1H-inden-2-yl)-5-methyl-1H-pyrazole-3-carboxamide,
1-[(5-Chloro-2-phenyl-1-benzofuran-7-yl)methyl]-N-[(2R)-2-hydroxypropyl]-5-methyl-1H-pyrazole-3-carboxamide,
1-[(5-Chloro-2-phenyl-1-benzofuran-7-yl)methyl]-N-[(2S)-2-hydroxypropyl]-5-methyl-1H-pyrazole-3-carboxamide,
1-[(5-Chloro-2-phenyl-1-benzofuran-7-yl)methyl]-5-methyl-N-(3,3,3-trifluoro-2-hydroxypropyl)-1H-pyrazole-3-carboxamide,
5-Methyl-1-{[5-(methylsulfonyl)-2-phenyl-1-benzofuran-7-yl]methyl}-N-(tetrahydro-2H-pyran-4-yl)-1H-pyrazole-3-carboxamide,
N-(1,1-Dimethylethyl)-5-methyl-1-{[5-(methylsulfonyl)-2-phenyl-1-benzofuran-7-yl]methyl}-1H-pyrazole-3-carboxamide,
5-Methyl-N-(1-methylethyl)-1-{[5-(methylsulfonyl)-2-phenyl-1-benzofuran-7-yl]methyl}-1H-pyrazole-3-carboxamide,
5-Methyl-N-(2-methylpropyl)-1-{[5-(methylsulfonyl)-2-phenyl-1-benzofuran-7-yl]methyl}-1H-pyrazole-3-carboxamide,
N-(Cyclopropylmethyl)-5-methyl-1-{[5-(methylsulfonyl)-2-phenyl-1-benzofuran-7-yl]methyl}-1H-pyrazole-3-carboxamide,
N-Cyclopropyl-5-methyl-1-{[5-(methylsulfonyl)-2-phenyl-1-benzofuran-7-yl]methyl}-1H-pyrazole-3-carboxamide,
N-Cyclobutyl-5-methyl-1-{[5-(methylsulfonyl)-2-phenyl-1-benzofuran-7-yl]methyl}-1H-pyrazole-3-carboxamide,
N-(Cyclobutylmethyl)-5-methyl-1-{[5-(methylsulfonyl)-2-phenyl-1-benzofuran-7-yl]methyl}-1H-pyrazole-3-carboxamide,
N-(2,2-Dimethylpropyl)-5-methyl-1-{[5-(methylsulfonyl)-2-phenyl-1-benzofuran-7-yl]methyl}-1H-pyrazole-3-carboxamide,
N-(2,3-Dihydro-1H-inden-2-yl)-5-methyl-1-{[5-(methylsulfonyl)-2-phenyl-1-benzofuran-7-yl]methyl}-1H-pyrazole-3-carboxamide,
N-[(1S)-2,3-Dihydro-1H-inden-1-yl]-5-methyl-1-{[5-(methylsulfonyl)-2-phenyl-1-benzofuran-7-yl]methyl}-1H-pyrazole-3-carboxamide,
N-[(1R)-2,3-Dihydro-1H-inden-1-yl]-5-methyl-1-{[5-(methylsulfonyl)-2-phenyl-1-benzofuran-7-yl]methyl}-1H-pyrazole-3-carboxamide,
N-{1-[(5-chloro-2-phenyl-1-benzofuran-7-yl)methyl]-5-methyl-1H-pyrazol-3-yl}-5-oxo-L-prolinamide,
N¹-{1-[(5-chloro-2-phenyl-1-benzofuran-7-yl)methyl]-5-methyl-1H-pyrazol-3-yl}-N²,N²-dimethylglycinamide,
N-{1-[(5-chloro-2-phenyl-1-benzofuran-7-yl)methyl]-5-methyl-1H-pyrazol-3-yl}-1-(2,2,2-trifluoroethyl)-4-piperidinecarboxamide,
N-{1-[(5-chloro-2-phenyl-1-benzofuran-7-yl)methyl]-5-methyl-1H-pyrazol-3-yl}-1-methyl-5-oxo-3-pyrrolidinecarboxamide,
N-{1-[(5-chloro-2-phenyl-1-benzofuran-7-yl)methyl]-5-methyl-1H-pyrazol-3-yl}-2-(2-oxo-1-pyrrolidinyl)acetamide,
(3R)—N-{1-[(5-chloro-2-phenyl-1-benzofuran-7-yl)methyl]-5-methyl-1H-pyrazol-3-yl}-1-(2,2,2-trifluoroethyl)-3-piperidinecarboxamide methane sulfonate,
N-{1-[(5-chloro-2-phenyl-1-benzofuran-7-yl)methyl]-5-methyl-1H-pyrazol-3-yl}tetrahydro-2H-pyran-4-carboxamide,
(2R)—N-{1-[(5-chloro-2-phenyl-1-benzofuran-7-yl]methyl}-5-methyl-1H-pyrazol-3-yl)tetrahydro-2-furancarboxamide,
N-{1-[(5-chloro-2-phenyl-1-benzofuran-7-yl)methyl]-5-methyl-1H-pyrazol-3-yl}-2-methylpropanamide,
N-(1-{[5-Chloro-2-(4-cyanophenyl)-1-benzofuran-7-yl]methyl}-5-methyl-1H-pyrazol-3-yl)-2-methylpropanamide,
(2R)—N-{1-[(5-chloro-2-(4-cyanophenyl)-1-benzofuran-7-yl)methyl]-5-methyl-1H-pyrazol-3-yl}tetrahydro-2-furancarboxamide,
N-{1-[(5-Chloro-2-(4-cyanophenyl)-1-benzofuran-7-yl]methyl}-5-methyl-1H-pyrazol-3-yl)tetrahydro-2H-pyran-4-carboxamide,
N-{1-[(5-Cyano-2-phenyl-1-benzofuran-7-yl)methyl]-5-methyl-1H-pyrazol-3-yl}tetrahydro-2H-pyran-4-carboxamide,
2-Methylpropyl {1-[(5-chloro-2-phenyl-1-benzofuran-7-yl)methyl]-5-methyl-1H-pyrazol-3-yl}carbamate,
N-{1-[(5-Chloro-2-phenyl-1-benzofuran-7-yl)methyl]-5-methyl-1H-pyrazol-3-yl}-N′-(1,1-dimethylethyl)urea, and
1,1-Dimethylethyl {5-[(5-chloro-2-phenyl-1-benzofuran-7-yl)methyl]-2-furanyl}carbamate,
or a pharmaceutically acceptable derivative thereof.

3. A pharmaceutical composition comprising a compound according to claim 1 or a pharmaceutically acceptable derivative thereof together with a pharmaceutical carrier, excipient, or both.

4.-5. (canceled)

6. A method of treating a human subject suffering from a condition which is mediated by the action of PGE₂ at EP₁ receptors which comprises administering to said subject an effective amount of a compound according to claim 1 or a pharmaceutically acceptable derivative thereof.

7. A method of treating a human subject suffering from a pain, or an inflammatory, immunological, bone, neurodegenerative or renal disorder, which method comprises administering to said subject an effective amount of a compound according to claim 1 or a pharmaceutically acceptable derivative thereof.

8. A method of treating a human subject suffering from inflammatory pain, neuropathic pain or visceral pain which method comprises administering to said subject an effective amount of a compound according to claim 1 or a pharmaceutically acceptable derivative thereof.

9.-11. (canceled)
